(19) United States
(12) Patent Application Publication
(10) Pub. No.: US 2011/0091924 A1
(43) Pub. Date: Apr. 21, 2011

(54) Method of evaluating cancer type

(75) Inventors: Akira Imaizumi, Kanagawa (JP); Toshihiko Ando, Kanagawa (JP); Naoyuki Okamoto, Kanagawa (JP); Fumio Imamura, Osaka (JP); Masahiko Higashiyama, Osaka (JP)

(73) Assignee: Ajinomoto Co., Inc.

(21) Appl. No.: 12/923,147

(22) Filed: Sep. 3, 2010

Related U.S. Application Data
(63) Continuation of application No. PCT/JP2009/054091, filed on Mar. 4, 2009.

(30) Foreign Application Priority Data
Mar. 4, 2008 (JP) 2008-054114

(51) Int. Cl. C12Q 1/02

(52) U.S. Cl. 435/29

(57) ABSTRACT

According to the method of evaluating cancer type of the present invention, amino acid concentration data on concentration values of amino acids in blood collected from a subject to be evaluated is measured, and the cancer type in the subject is evaluated based on the concentration value of at least one of Glu, ABA, Val, Met, Pro, Phe, Thr, Ile, Leu, and His contained in the measured amino acid concentration data of the subject.

This application is a Continuation of PCT/JP2009/054091, filed Mar. 4, 2009, which claims priority from Japanese patent application JP 2008-054114 filed Mar. 4, 2008. The contents of each of the aforementioned application are incorporated herein by reference in their entirety.

### BACKGROUND OF THE INVENTION

1. Field of the Invention

The present invention relates to a method of evaluating cancer type, which utilizes a concentration of an amino acid in blood (plasma).

2. Description of the Related Art

The number of deaths from cancer in Japan in 2004 is 193075 males and 127259 females, and the number of deaths ranks first among the total numbers of deaths. The survival rate may be dependent on the type of cancer, but there are some types for which the five-year survival rate of early stage cancer is 80% or higher, while there are also some types for which the five-year survival rate of progressive cancer is extremely low, such as about 10%. Therefore, early detection is important for treatment of cancer.

Here, diagnosis of colon cancer includes, for example, diagnosis based on the immunological fecal occult blood reaction, and colon biopsy by colonoscopy.

However, diagnosis based on a fecal occult blood test does not serve as definitive diagnosis, and most of the persons with positive-finding are false-positive. Furthermore, in regard to early colon cancer, there is a concern that both the detection sensitivity and the detection specificity become lower in the diagnosis based on a fecal occult blood test. In particular, early cancer in the right side colon is frequently overlooked when diagnosed by a fecal occult blood test. Diagnostic imaging by CT (computer tomography), MRI (magnetic resonance imaging), PET (positron emission computerized-tomography) or the like is not suitable for the diagnosis of colon cancer.

On the other hand, colon biopsy by colonoscopy serves as definitive diagnosis, but is a highly invasive examination, and implementing colon biopsy at the screening stage is not practical. Furthermore, invasive diagnosis such as colon biopsy gives a burden to patients, such as accompanying pain, and there may also be a risk of bleeding upon examination, or the like.

Therefore, from the viewpoints of a physical burden imposed on patients and of cost-benefit performance, it is desirable to narrow down the target range of test subjects with high possibility of onset of colon cancer, and to subject those people to treatment. Specifically, it is desirable that test subjects are selected by a less invasive method, the target range of the selected test subjects is narrowed by subjecting the selected test subjects to a colonoscopic examination, and the test subjects who are definitively diagnosed as having colon cancer are subjected to treatment.

For another example, diagnosis of lung cancer includes diagnosis by imaging with X-ray picture, CT, MRI, PET or the like, sputum cytodiagnosis, lung biopsy with a bronchoscope, lung biopsy with a percutaneous needle, lung biopsy by exploratory thoracotomy or with a thoracoscope, and the like.

However, diagnosis by imaging does not serve as definitive diagnosis. For example, in chest X-ray examination (indirect roentgenography), the positive-finding rate is 20%, while the specificity is 0.1%, and most of the persons with positive-finding are false-positive. Furthermore, in the case of chest X-ray examination, the detection sensitivity is low, and some examination results according to the Ministry of Health, Labour and Welfare of Japan also report that about 80% of patients who developed lung cancer were overlooked. Particularly, in early lung cancer, there is a concern that diagnosis by imaging is even poorer in both detection sensitivity and detection specificity. In chest X-ray examination, there is also a problem of exposure of test subjects to radiation. Diagnostic imaging by CT, MRI, PET or the like also is not suitable to be carried out as mass screening, from the viewpoints of facilities and costs. In the case of sputum cytodiagnosis, only 20 to 30% of patients can be diagnosed definitively.

On the other hand, lung biopsy using a bronchoscope, a percutaneous needle, exploratory thoracotomy or a thoracoscope serves as definitive diagnosis, but is a highly invasive examination, and implementing lung biopsy on all patients who are suspected of having lung cancer as a result of diagnostic imaging, is not practical. Furthermore, such invasive diagnosis gives a burden to patients, such as accompanying pain, and there may also be a risk of bleeding upon examination, or the like.

Therefore, from the viewpoints of a physical burden imposed on patients and of cost-benefit performance, it is desirable to narrow down the target range of test subjects with high possibility of onset of lung cancer, and to subject those people to treatment. Specifically, it is desirable that test subjects are selected by a less invasive method, the target range of the selected test subjects is narrowed by subjecting the selected test subjects to lung biopsy, and the test subjects who are definitively diagnosed as having lung cancer are subjected to treatment.

For another example, diagnosis of breast cancer includes self examination, breast palpation and visual inspection, diagnostic imaging by mammography, CT, MRI, PET or the like, needle biopsy, and the like.

However, self examination, palpation and visual inspection, and diagnostic imaging do not serve as definitive diagnosis. In particular, self examination is not effective to the extent of lowering the rate of deaths from breast cancer. Furthermore, self examination does not enable the discovery of a large number of early cancers, as regular screening by a mammographic examination does. In early breast cancer, there is a concern that self examination, palpation and visual inspection, or diagnostic imaging is even poorer in both detection sensitivity and detection specificity. Diagnostic imaging by mammography also has a problem of exposure of test subject to radiation or overdiagnosis. Diagnostic imaging by CT, MRI, PET or the like also is not suitable to be carried out as mass screening, from the viewpoints of facilities and costs.

On the other hand, needle biopsy serves as definitive diagnosis, but is a highly invasive examination, and implementing needle biopsy on all patients who are suspected of having breast cancer as a result of diagnostic imaging, is not practical. Furthermore, such invasive diagnosis as needle biopsy gives a burden to patients, such as accompanying pain, and there may also be a risk of bleeding upon examination, or the like.

Generally, it is conceived that in many cases excluding self examination, examination of breast cancer makes test subjects hesitating.

Therefore, from the viewpoints of a physical burden and a mental burden imposed on test subjects, and of cost-benefit performance, it is desirable to narrow down the target range of test subjects with high possibility of onset of breast cancer, and to subject those people to treatment. Specifically, it is desirable that test subjects are selected by a method accompanied with less mental suffering or a less invasive method, the target range of the selected test subjects is narrowed by subjecting the selected test subjects to needle biopsy, and the test subjects who are definitively diagnosed as having breast cancer are subjected to treatment.

For another example, diagnosis of gastric cancer includes a pepsinogen test, X-ray examination (indirect roentgenography), gastroscopic examination, diagnosis with a tumor marker, and the like.

However, a pepsinogen test, X-ray examination, and diagnosis with a tumor marker do not serve as definitive diagnosis. For example, the pepsinogen test is less invasive, but the sensitivity varies in different reports, approximately from 40 to 85%, while the specificity is 70 to 85%. However, in the case of the pepsinogen test, the rate of recall for thorough examination is 20%, and it is conceived that the results are frequently overlooked. In the case of X-ray examination, the sensitivity varies in different reports, approximately from 70 to 80%, while the specificity is 85 to 90%. However, the X-ray examination has a possibility of causing adverse side effects due to the drinking of barium, or of exposure to radiation. In the case of diagnosis with a tumor marker, a tumor marker which is effective for diagnosing the presence of gastric cancer does not exist at present.

On the other hand, gastroscopic examination serves as definitive diagnosis, but is a highly invasive examination, and implementing gastroscopic examination at the screening stage is not practical. Furthermore, invasive diagnosis such as gastroscopic examination gives a burden to patients, such as accompanying pain, and there may also be a risk of bleeding upon examination, or the like.

Therefore, from the viewpoints of a physical burden imposed on patients and of cost-benefit performance, it is desirable to narrow down the target range of test subjects with high possibility of onset of gastric cancer, and to subject those people to treatment. Specifically, it is desirable that test subjects are selected by a method having high sensitivity and specificity, the target range of the selected test subjects is narrowed by subjecting the selected test subjects to gastroscopic examination, and the test subjects who are definitively diagnosed as having gastric cancer are subjected to treatment.

Furthermore, there are also cancers which are difficult to detect early, such as pancreatic cancer.

In the case of pancreatic cancer, after a patient complains of subjective symptoms, the patient is diagnosed definitively as pancreatic cancer by thorough examination, but in many cases, cancer is diagnosed as progressive cancer.

Therefore, from the viewpoints of a physical burden imposed on patients and of cost-benefit performance, it is desirable to narrow down the target range of test subjects with high possibility of onset of pancreatic cancer by appropriate screening, and to subject those people to treatment. Specifically, it is desirable that test subjects are selected by a method having high sensitivity and specificity, the target range of the selected test subjects is narrowed by subjecting the selected test subjects to thorough examination, and the test subjects who are definitively diagnosed as having pancreatic cancer are subjected to treatment.

Such screening of cancer patients is currently carried out using a specific diagnosis approach to each cancer.

Incidentally, it is known that the concentrations of amino acids in blood change as a result of onset of cancer. For example, Cynober (Cynober, L. ed., Metabolic and therapeutic aspects of amino acids in clinical nutrition. 2nd ed., CRC Press.) has reported that, for example, the amount of consumption increases in cancer cells, for glutamine mainly as an oxidation energy source, for arginine as a precursor of nitrogen oxide and polyamine, and for methionine through the activation of the ability of cancer cells to take in methionine, respectively. Vissers, et al. (Vissers, Y. L J., et. al., Plasma arginine concentration are reduced in cancer patients: evidence for arginine deficiency?, The American Journal of Clinical Nutrition, 2005 81, p. 1142-1146) and Park (Park, K. G., et al., Arginine metabolism in benign and malignant disease of breast and colon: evidence for possible inhibition of tumor-infiltrating macrophages., Nutrition, 1991 7, p. 185-188) have reported that the amino acid composition in plasma in colon cancer patients is different from that of healthy individuals. Proenza, et al. (Proenza, A. M., J. Oliver, A. Palou and P. Roca, Breast and lung cancer are associated with a decrease in blood cell amino acid content. J Nutr Biochem, 2003. 14(3): p. 133-8.) and Cascino (Cascino, A., M. Muscaritoli, C. Cangiano, L. Conversano, A. Laviano, S. Ariemma, M. M. Meguid and F. Rossi Fanelli, Plasma amino acid imbalance in patients with lung and breast cancer. Anticancer Res, 1995. 15(2): p. 507-10.) have reported that the amino acid composition in plasma in breast cancer patients is different from that of healthy individuals. WO 2008/016111 discloses a method of evaluating the presence or absence of lung cancer by multivariate discriminants with the concentration of amino acids in blood as explanatory variables. Thus, state of lung cancer or lung cancer-free can be discriminated. WO 2004/052191 and WO 2006/098192 disclose a method of associating amino acid concentration with biological state.

However, there is a problem that the development of techniques of diagnosing a cancer type with a plurality of amino acids as explanatory variables is not conducted from the viewpoint of time and cost and is not practically used. Specifically, when carrying out a plurality of examinations at the same time in the screening of cancer patients, there is a problem that an examination cost becomes high and an examinee is restrained for a long time and time of diet restriction and the like becomes long according to contents thereof. Specifically, WO 2008/016111 has a problem that although the state of lung cancer or lung cancer-free may be discriminated, it is not possible to evaluate whether “the state of lung cancer-free is without cancer” and whether “the state is with another cancer”. In the index formula disclosed in WO 2004/052191 and WO 2006/098192, there is a problem that it is not possible to evaluate whether “the state is without the cancer” and whether “the state is with another cancer”.

### SUMMARY OF THE INVENTION

It is an object of the present invention to at least partially solve the problems in the conventional technology. The present invention is made in view of the problem described above, and an object of the present invention is to provide a method of evaluating cancer type, which is capable of evaluating the cancer type accurately by utilizing the concentration of the amino acid related to states of various cancers among amino acids in blood. Specifically, an object thereof is to provide the method of evaluating cancer type, which is capable of narrowing an examinee likely to contract a plurality of cancers by one sample in a short time, thereby reducing temporal, physical and financial burden of the examinee. Specifically, an object thereof is to provide the method of evaluating cancer type, which is capable of evaluating accurately whether a certain sample is with cancer and where an affected area is when this is with the cancer, by the concentrations of a plurality of the amino acids and a discriminant group composed of one or a plurality of discriminants with the concentrations of the amino acids as explanatory variables, thereby making the examination efficient and high accurate.

The present inventors have made extensive study for solving the problem described above, and as a result they have identified amino acids which are useful in multiple-group discrimination among various cancers and cancer-free, and have found that a multivariate discriminant group (index formula group, correlation equation group) composed of one or a plurality of multivariate discriminants containing the concentrations of the identified amino acids as the explanatory variables correlates significantly with the state of cancer (specifically, site of onset of cancer), and the present invention is thereby completed.

To solve the problem and achieve the object described above, a method of evaluating cancer type according to one aspect of the present invention includes a measuring step of measuring amino acid concentration data on a concentration value of an amino acid in blood collected from a subject to be evaluated, and a concentration value criterion evaluating step of evaluating a cancer type in the subject based on the concentration value of at least one of Glu, ABA, Val, Met, Pro, Phe, Thr, Ile, Leu, and His contained in the amino acid concentration data of the subject measured at the measuring step.

Another aspect of the present invention is the method of evaluating cancer type, wherein the concentration value criterion evaluating step further includes a concentration value criterion discriminating step of discriminating a cancer in the subject out of at least two of colon cancer, breast cancer, prostatic cancer, thyroid cancer, lung cancer, gastric cancer, and uterine cancer based on the concentration value of at least one of Glu, ABA, Val, Met, Pro, Phe, Thr, Ile, Leu, and His contained in the amino acid concentration data of the subject measured at the measuring step.

Still another aspect of the present invention is the method of evaluating cancer type, wherein at the concentration value criterion discriminating step, the cancer in the subject is discriminated out of at least three of colon cancer, breast cancer, prostatic cancer, thyroid cancer, and lung cancer.

Still another aspect of the present invention is the method of evaluating cancer type, wherein the concentration value criterion evaluating step further includes (i) a discriminant value calculating step of calculating a discriminant value that is a value of a multivariate discriminant with a concentration of the amino acid as an explanatory variable, for each of the multivariate discriminants composing a multivariate discriminant group, based on both (a) the concentration value of at least one of Glu, ABA, Val, Met, Pro, Phe, Thr, Ile, Leu, and His contained in the amino acid concentration data of the subject measured at the measuring step and (b) the multivariate discriminant group composed of one or a plurality of the previously established multivariate discriminants, and (ii) a discriminant value criterion evaluating step of evaluating the cancer type in the subject based on a discriminant value group composed of one or a plurality of the discriminant values calculated at the discriminant value calculating step. Each of the multivariate discriminants composing the multivariate discriminant group contains at least one of Glu, ABA, Val, Met, Pro, Phe, Thr, Ile, Leu, and His as the explanatory variable.

Still another aspect of the present invention is the method of evaluating cancer type, wherein the discriminant value criterion evaluating step further includes a discriminant value criterion discriminating step of discriminating the cancer in the subject out of at least two of colon cancer, breast cancer, prostatic cancer, thyroid cancer, lung cancer, gastric cancer, and uterine cancer based on the discriminant value group.

Still another aspect of the present invention is the method of evaluating cancer type, wherein at the discriminant value criterion discriminating step, the cancer in the subject is discriminated out of at least three of colon cancer, breast cancer, prostatic cancer, thyroid cancer, and lung cancer.

Still another aspect of the present invention is the method of evaluating cancer type, wherein each of the multivariate discriminants composing the multivariate discriminant group is any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the method of evaluating cancer type, wherein the multivariate discriminant group is any one of following discriminant groups 1 to 16.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

The present invention also relates to a cancer type-evaluating apparatus, the cancer type-evaluating apparatus according to one aspect of the present invention includes a control unit and a memory unit to evaluate a cancer type in a subject to be evaluated. The control unit includes (i) a discriminant value-calculating unit that calculates a discriminant value that is a value of a multivariate discriminant with a concentration of an amino acid as an explanatory variable, for each of the multivariate discriminants composing a multivariate discriminant group, based on both (a) a concentration value of at least one of Glu, ABA, Val, Met, Pro, Phe, Thr, Ile, Leu, and His contained in a previously obtained amino acid concentration data of the subject on the concentration value of the amino acid and (b) the multivariate discriminant group composed of one or a plurality of the multivariate discriminants stored in the memory unit, and (ii) a discriminant value criterion-evaluating unit that evaluates the cancer type in the subject based on a discriminant value group composed of one or a plurality of the discriminant values calculated by the discriminant value-calculating unit. Each of the multivariate discriminants composing the multivariate discriminant group contains at least one of Glu, ABA, Val, Met, Pro, Phe, Thr, Ile, Leu, and His as the explanatory variable.

Another aspect of the present invention is the cancer type-evaluating apparatus, wherein the discriminant value criterion-evaluating unit further includes a discriminant value criterion-discriminating unit that discriminates a cancer in the subject out of at least two of colon cancer, breast cancer, prostatic cancer, thyroid cancer, lung cancer, gastric cancer, and uterine cancer based on the discriminant value group.

Still another aspect of the present invention is the cancer type-evaluating apparatus, wherein the discriminant value criterion-discriminating unit discriminates the cancer in the subject out of at least three of colon cancer, breast cancer, prostatic cancer, thyroid cancer, and lung cancer.

Still another aspect of the present invention is the cancer type-evaluating apparatus, wherein each of the multivariate discriminants composing the multivariate discriminant group is any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the cancer type-evaluating apparatus, wherein the multivariate discriminant group is any one of following discriminant groups 1 to 16.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Still another aspect of the present invention is the cancer type-evaluating apparatus, wherein the control unit further includes a multivariate discriminant group-preparing unit that prepares the multivariate discriminant stored in the memory unit, based on cancer state information containing the amino acid concentration data and cancer state index data on an index for indicating a cancer state, stored in the memory unit. The multivariate discriminant group-preparing unit further includes (i) a candidate multivariate discriminant group-preparing unit that prepares a candidate multivariate discriminant group that is a candidate of the multivariate discriminant group, based on a predetermined discriminant-preparing method from the cancer state information, (ii) a candidate multivariate discriminant group-verifying unit that verifies the candidate multivariate discriminant group prepared by the candidate multivariate discriminant group-preparing unit, based on a predetermined verifying method, and (iii) an explanatory variable-selecting unit that selects the explanatory variable of the candidate multivariate discriminant group based on a predetermined explanatory variable-selecting method from a verification result obtained by the candidate multivariate discriminant group-verifying unit, thereby selecting a combination of the amino acid concentration data contained in the cancer state information used in preparing the candidate multivariate discriminant group. The multivariate discriminant group-preparing unit prepares the multivariate discriminant group by selecting the candidate multivariate discriminant group used as the multivariate discriminant group, from a plurality of the candidate multivariate discriminant groups, based on the verification results accumulated by repeatedly executing the candidate multivariate discriminant group-preparing unit, the candidate multivariate discriminant group-verifying unit, and the explanatory variable-selecting unit.

The present invention also relates to a cancer type-evaluating method, one aspect of the present invention is the cancer type-evaluating method of evaluating a cancer type in a subject to be evaluated. The method is carried out with an information processing apparatus including a control unit and a memory unit. The method includes (i) a discriminant value calculating step of calculating a discriminant value that is a value of a multivariate discriminant with a concentration of an amino acid as an explanatory variable, for each of the multivariate discriminants composing a multivariate discriminant group, based on both (a) a concentration value of at least one of Glu, ABA, Val, Met, Pro, Phe, Thr, Ile, Leu, and His contained in a previously obtained amino acid concentration data of the subject on the concentration value of the amino acid and (b) the multivariate discriminant group composed of one or a plurality of the multivariate discriminants stored in the memory unit, and (ii) a discriminant value criterion evaluating step of evaluating the cancer type in the subject based on a discriminant value group composed of one or a plurality of the discriminant values calculated at the discriminant value calculating step. Each of the multivariate discriminants composing the multivariate discriminant group contains at least one of Glu, ABA, Val, Met, Pro, Phe, Thr, Ile, Leu, and His as the explanatory variable. The steps (i) and (ii) are executed by the control unit.

Another aspect of the present invention is the cancer type-evaluating method, wherein the discriminant value criterion evaluating step further includes a discriminant value criterion discriminating step of discriminating a cancer in the subject out of at least two of colon cancer, breast cancer, prostatic cancer, thyroid cancer, lung cancer, gastric cancer, and uterine cancer based on the discriminant value group.

Still another aspect of the present invention is the cancer type-evaluating method, wherein at the discriminant value criterion discriminating step, the cancer in the subject is discriminated out of at least three of colon cancer, breast cancer, prostatic cancer, thyroid cancer, and lung cancer.

Still another aspect of the present invention is the cancer type-evaluating method, wherein each of the multivariate discriminants composing the multivariate discriminant group is any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

Still another aspect of the present invention is the cancer type-evaluating method, wherein the multivariate discriminant group is any one of following discriminant groups 1 to 16.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Still another aspect of the present invention is the cancer type-evaluating method, wherein the method further includes a multivariate discriminant preparing step of preparing the multivariate discriminant stored in the memory unit, based on cancer state information containing the amino acid concentration data and cancer state index date on an index for indicating a cancer state, stored in the memory unit that is executed by the control unit. The multivariate discriminant preparing step further includes (i) a candidate multivariate discriminant preparing step of preparing a candidate multivariate discriminant that is a candidate of the multivariate discriminant, based on a predetermined discriminant-preparing method from the cancer state information, (ii) a candidate multivariate discriminant verifying step of verifying the candidate multivariate discriminant prepared at the candidate multivariate preparing step, based on a predetermined verifying method, and (iii) an explanatory variable selecting step of selecting the explanatory variable of the candidate multivariate discriminant based on a predetermined explanatory variable-selecting method from a verification result obtained at the candidate multivariate discriminant verifying step, thereby selecting a combination of the amino acid concentration data contained in the cancer state information used in preparing the candidate multivariate discriminant. At the multivariate discriminant preparing step, the multivariate discriminant is prepared by selecting the candidate multivariate discriminant used as the multivariate discriminant, from a plurality of the candidate multivariate discriminants, based on the verification results accumulated by repeatedly executing the candidate multivariate discriminant preparing step, the candidate multivariate discriminant verifying step, and the explanatory variable selecting step.

The present invention also relates to a cancer type-evaluating system, the cancer type-evaluating system according to one aspect of the present invention includes a cancer type-evaluating apparatus including a control unit and a memory unit to evaluate a cancer type in a subject to be evaluated, and an information communication terminal apparatus that provides amino acid concentration data of the subject on a concentration value of an amino acid. The apparatuses are connected to each other communicatively via a network. The information communication terminal apparatus includes an amino acid concentration data-sending unit that transmits the amino acid concentration data of the subject to the cancer type-evaluating apparatus, and an evaluation result-receiving unit that receives an evaluation result of the subject on the cancer type transmitted from the cancer type-evaluating apparatus. The control unit of the cancer type-evaluating apparatus includes (i) an amino acid concentration data-receiving unit that receives the amino acid concentration data of the subject transmitted from the information communication terminal apparatus, (ii) a discriminant value-calculating unit that calculates a discriminant value that is a value of a multivariate discriminant with a concentration of the amino acid as an explanatory variable, for each of the multivariate discriminants composing a multivariate discriminant group, based on both (a) the concentration value of at least one of Glu, ABA, Val, Met, Pro, Phe, Thr, Ile, Leu, and His contained in the amino acid concentration data of the subject received by the amino acid concentration data-receiving unit and (b) the multivariate discriminant group composed of one or a plurality of the multivariate discriminants stored in the memory unit, (iii) a discriminant value criterion-evaluating unit that evaluates the cancer type in the subject based on a discriminant value group composed of one or a plurality of the discriminant values calculated by the discriminant value-calculating unit, and (iv) an evaluation result-sending unit that transmits the evaluation result of the subject obtained by the discriminant value criterion-evaluating unit to the information communication terminal apparatus. Each of the multivariate discriminants composing the multivariate discriminant group contains at least one of Glu, ABA, Val, Met, Pro, Phe, Thr, Ile, Leu, and His as the explanatory variable.

The present invention also relates to a cancer type-evaluating program product, one aspect of the present invention is the cancer type-evaluating program product that makes an information processing apparatus including a control unit and a memory unit execute a method of evaluating a cancer type in a subject to be evaluated. The method includes (i) a discriminant value calculating step of calculating a discriminant value that is a value of a multivariate discriminant with a concentration of an amino acid as an explanatory variable, for each of the multivariate discriminants composing a multivariate discriminant group, based on both (a) a concentration value of at least one of Glu, ABA, Val, Met, Pro, Phe, Thr, Ile, Leu, and His contained in a previously obtained amino acid concentration data of the subject on the concentration value of the amino acid and (b) the multivariate discriminant group composed of one or a plurality of the multivariate discriminants stored in the memory unit, and (ii) a discriminant value criterion evaluating step of evaluating the cancer type in the subject based on a discriminant value group composed of one or a plurality of the discriminant values calculated at the discriminant value calculating step. Each of the multivariate discriminants composing the multivariate discriminant group contains at least one of Glu, ABA, Val, Met, Pro, Phe, Thr, Ile, Leu, and His as the explanatory variable. The steps (i) and (ii) are executed by the control unit.

The present invention also relates to a recording medium, the recording medium according to one aspect of the present invention includes the cancer type-evaluating program product described above.

According to the present invention, (i) the amino acid concentration data on the concentration value of the amino acid in blood collected from the subject is measured, and (ii) the cancer type in the subject is evaluated based on the concentration value of at least one of Glu, ABA, Val, Met, Pro, Phe, Thr, Ile, Leu, and His contained in the measured amino acid concentration data of the subject. Thus, concentrations of amino acids which among amino acids in blood, are related to states of various cancers can be utilized to bring about an effect of enabling an accurate evaluation of the cancer type. Specifically, an examinee likely to contract a plurality of cancers can be narrowed by one sample in a short time to bring about an effect of enabling a reduction of temporal, physical and financial burden of the examinee. Specifically, whether a certain sample is with cancer and where an affected area is when this is with the cancer can be evaluated accurately by concentrations of a plurality of amino acids and a discriminant group composed of one or a plurality of discriminants with the concentrations of the amino acids as the explanatory variables to bring about an effect of enabling to make the examination efficient and high accurate.

According to the present invention, the cancer in the subject is discriminated out of at least two of colon cancer, breast cancer, prostatic cancer, thyroid cancer, lung cancer, gastric cancer, and uterine cancer based on the concentration value of at least one of Glu, ABA, Val, Met, Pro, Phe, Thr, Ile, Leu, and His contained in the measured amino acid concentration data of the subject. Thus, concentrations of amino acids which among amino acids in blood, are useful for a multiple-group discrimination of cancer can be utilized to bring about an effect of enabling accurately the multiple-group discrimination of cancer.

According to the present invention, the cancer in the subject is discriminated out of at least three of colon cancer, breast cancer, prostatic cancer, thyroid cancer, and lung cancer based on the concentration value of at least one of Glu, ABA, Val, Met, Pro, Phe, Thr, Ile, Leu, and His contained in the measured amino acid concentration data of the subject. Thus, concentrations of amino acids which among amino acids in blood, are useful for a multiple-group discrimination of cancer can be utilized to bring about an effect of enabling accurately the multiple-group discrimination of cancer.

According to the present invention, (i) the discriminant value that is the value of the multivariate discriminant with the concentration of the amino acid as the explanatory variable is calculated for each of the multivariate discriminants composing the multivariate discriminant group, based on both (a) the concentration value of at least one of Glu, ABA, Val, Met, Pro, Phe, Thr, Ile, Leu, and His contained in the measured amino acid concentration data of the subject and (b) the multivariate discriminant group composed of one or a plurality of the previously established multivariate discriminants containing at least one of Glu, ABA, Val, Met, Pro, Phe, Thr, Ile, Leu, and His as the explanatory variable, and (ii) the cancer type in the subject is evaluated based on the discriminant value group composed of one or a plurality of the calculated discriminant values. Thus, a discriminant value group obtained in a multivariate discriminant group correlated significantly with states of various cancers can be utilized to bring about an effect of enabling an accurate evaluation of the cancer type. Specifically, an examinee likely to contract a plurality of cancers can be narrowed by one sample in a short time to bring about an effect of enabling a reduction of temporal, physical and financial burden of the examinee. Specifically, whether a certain sample is with cancer and where an affected area is when this is with the cancer can be evaluated accurately by concentrations of a plurality of amino acids and a discriminant group composed of one or a plurality of discriminants with the concentrations of the amino acids as the explanatory variables to bring about an effect of enabling to make the examination efficient and high accurate.

According to the present invention, the cancer in the subject is discriminated out of at least two of colon cancer, breast cancer, prostatic cancer, thyroid cancer, lung cancer, gastric cancer, and uterine cancer based on the calculated discriminant value group. Thus, a discriminant value group obtained in a multivariate discriminant group useful for a multiple-group discrimination of cancer can be utilized to bring about an effect of enabling accurately the multiple-group discrimination of cancer.

According to the present invention, the cancer in the subject is discriminated out of at least three of colon cancer, breast cancer, prostatic cancer, thyroid cancer, and lung cancer based on the calculated discriminant value group. Thus, a discriminant value group obtained in a multivariate discriminant group useful for a multiple-group discrimination of cancer can be utilized to bring about an effect of enabling accurately the multiple-group discrimination of cancer.

According to the present invention, each of the multivariate discriminants composing the multivariate discriminant group is any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Thus, a discriminant value group obtained in a multivariate discriminant group useful particularly for a multiple-group discrimination of cancer can be utilized to bring about an effect of enabling more accurately the multiple-group discrimination of cancer.

According to the present invention, the multivariate discriminant group is any one of following discriminant groups 1 to 16. Thus, a discriminant value group obtained in a multivariate discriminant group useful particularly for a multiple-group discrimination of cancer can be utilized to bring about an effect of enabling more accurately the multiple-group discrimination of cancer.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

According to the present invention, the multivariate discriminant stored in the memory unit is prepared based on the cancer state information containing the amino acid concentration data and the cancer state index data on the index for indicating the cancer state, stored in the memory unit. Specifically, (1) the candidate multivariate discriminant is prepared based on the predetermined discriminant-preparing method from the cancer state information, (2) the prepared candidate multivariate discriminant is verified based on the predetermined verifying method, (3) the explanatory variables of the candidate multivariate discriminant are selected based on the predetermined explanatory variable-selecting method from the verification results, thereby selecting the combination of the amino acid concentration data contained in the cancer state information used in preparing of the candidate multivariate discriminant, and (4) the candidate multivariate discriminant used as the multivariate discriminant is selected from a plurality of the candidate multivariate discriminants based on the verification results accumulated by repeatedly executing (1), (2) and (3), thereby preparing the multivariate discriminant. Thus, a multivariate discriminant most appropriate for evaluating each cancer state can be prepared to bring about an effect of enabling to obtain a multivariate discriminant group most appropriate for evaluating the cancer type (specifically, the multivariate discriminant group useful for the multiple-group discrimination of cancer).

According to the present invention, the cancer type-evaluating program recorded on the recording medium is read and executed by the computer, thereby allowing the computer to execute the cancer type-evaluating program, thus bringing about an effect of obtaining the same effect as in the cancer type-evaluating program.

When the cancer type is evaluated (specifically, which of the cancers the subject has is discriminated) in the present invention, concentrations of other metabolites, gene expression level, protein expression level, age and sex of the subject, presence or absence of smoking, digitalized electrocardiogram waveform, or the like may be used in addition to the amino acid concentration. When the cancer type is evaluated (specifically, which of the cancers the subject has is discriminated) in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of the present invention;

FIG. 2 is a flowchart showing one example of a method of evaluating cancer type according to a first embodiment;

FIG. 3 is a principle configurational diagram showing a basic principle of the present invention;

FIG. 4 is a diagram showing an example of an entire configuration of a present system;

FIG. 5 is a diagram showing another example of an entire configuration of the present system;

FIG. 6 is a block diagram showing an example of a configuration of a cancer type-evaluating apparatus 100 in the present system;

FIG. 7 is a chart showing an example of information stored in a user information file 106a;

FIG. 8 is a chart showing an example of information stored in an amino acid concentration data file 106b;

FIG. 9 is a chart showing an example of information stored in a cancer state information file 106c;

FIG. 10 is a chart showing an example of information stored in a designated cancer state information file 106d;

FIG. 11 is a chart showing an example of information stored in a candidate multivariable discriminant file 106e1;

FIG. 12 is a chart showing an example of information stored in a verification result file 106e2;

FIG. 13 is a chart showing an example of information stored in a selected cancer state information file 106e3;

FIG. 14 is a chart showing an example of information stored in a multivariable discriminant file 106e4;

FIG. 15 is a chart showing an example of information stored in a discriminant value file 106f;

FIG. 16 is a chart showing an example of information stored in an evaluation result file 106g;

FIG. 17 is a block diagram showing a configuration of a multivariable discriminant-preparing part 102h;

FIG. 18 is a block diagram showing a configuration of a discriminant value criterion-evaluating part 102j;

FIG. 19 is a block diagram showing an example of a configuration of a client apparatus 200 in the present system;

FIG. 20 is a block diagram showing an example of a configuration of a database apparatus 400 in the present system;

FIG. 21 is a flowchart showing an example of a cancer type evaluation service processing performed in the present system;

FIG. 22 is a flowchart showing an example of a multivariate discriminant-preparing processing performed in the cancer type-evaluating apparatus 100 in the present system;

FIG. 23 is boxplots showing distributions of amino acid explanatory variables in male various cancer patients and male cancer-free subjects;

FIG. 24 is boxplots showing distributions of amino acid explanatory variables in female various cancer patients and female cancer-free subjects;

FIG. 25 is a chart showing p-values in one-way analysis of variance;

FIG. 26 is a chart showing explanatory variables in an index formula group 1 and coefficients of those;

FIG. 27 is a chart showing correct answer rates in various cancers and cancer-free;

FIG. 28 is a chart showing a list of discriminant groups having the same discrimination performance as that of the index formula group 1;

FIG. 29 is a chart showing a list of discriminant groups having the same discrimination performance as that of the index formula group 1;

FIG. 30 is a chart showing explanatory variables in an index formula group 2 and coefficients of those;

FIG. 31 is a chart showing correct answer rates in various cancers and cancer-free;

FIG. 32 is a chart showing a list of discriminant groups having the same discrimination performance as that of the index formula group 2;

FIG. 33 is a chart showing a list of discriminant groups having the same discrimination performance as that of the index formula group 2;

FIG. 34 is a chart showing explanatory variables in an index formula group 3 and coefficients of those;

FIG. 35 is a chart showing correct answer rates in various cancers and cancer-free;

FIG. 36 is a chart showing a list of discriminant groups having the same discrimination performance as that of the index formula group 3;

FIG. 37 is a chart showing a list of discriminant groups having the same discrimination performance as that of the index formula group 3;

FIG. 38 is a chart showing explanatory variables in an index formula group 4 and coefficients of those;

FIG. 39 is a chart showing correct answer rates in various cancers;

FIG. 40 is a chart showing a list of discriminant groups having the same discrimination performance as that of the index formula group 4;

FIG. 41 is a chart showing a list of discriminant groups having the same discrimination performance as that of the index formula group 4;

FIG. 42 is a chart showing explanatory variables in an index formula group 5 and coefficients of those;

FIG. 43 is a chart showing correct answer rates in various cancers;

FIG. 44 is a chart showing a list of discriminant groups having the same discrimination performance as that of the index formula group 5;

FIG. 45 is a chart showing a list of discriminant groups having the same discrimination performance as that of the index formula group 5;

FIG. 46 is a chart showing explanatory variables in an index formula group 6 and coefficients of those;

FIG. 47 is a chart showing correct answer rates in various cancers;

FIG. 48 is a chart showing a list of discriminant groups having the same discrimination performance as that of the index formula group 6;

FIG. 49 is a chart showing a list of discriminant groups having the same discrimination performance as that of the index formula group 6;

FIG. 50 is a chart showing explanatory variables in an index formula group 7 and coefficients of those;

FIG. 51 is a chart showing correct answer rates in various cancers and cancer-free;

FIG. 52 is a chart showing a list of discriminant groups having the same discrimination performance as that of the index formula group 7;

FIG. 53 is a chart showing a list of discriminant groups having the same discrimination performance as that of the index formula group 7;

FIG. 54 is a chart showing explanatory variables in an index formula group 8 and coefficients of those;

FIG. 55 is a chart showing correct answer rates in various cancers and cancer-free;

FIG. 56 is a chart showing a list of discriminant groups having the same discrimination performance as that of the index formula group 8;

FIG. 57 is a chart showing a list of discriminant groups having the same discrimination performance as that of the index formula group 8;

FIG. 58 is a chart showing explanatory variables in an index formula group 9 and coefficients of those;

FIG. 59 is a chart showing correct answer rates in various cancers and cancer-free;

FIG. 60 is a chart showing a list of discriminant groups having the same discrimination performance as that of the index formula group 9;

FIG. 61 is a chart showing a list of discriminant groups having the same discrimination performance as that of the index formula group 9;

FIG. 62 is a chart showing explanatory variables in an index formula group 10 and coefficients of those;

FIG. 63 is a chart showing correct answer rates in various cancers;

FIG. 64 is a chart showing a list of discriminant groups having the same discrimination performance as that of the index formula group 10;

FIG. 65 is a chart showing a list of discriminant groups having the same discrimination performance as that of the index formula group 10;

FIG. 66 is a chart showing explanatory variables in an index formula group 11 and coefficients of those;

FIG. 67 is a chart showing correct answer rates in various cancers;

FIG. 68 is a chart showing a list of discriminant groups having the same discrimination performance as that of the index formula group 11;

FIG. 69 is a chart showing a list of discriminant groups having the same discrimination performance as that of the index formula group 11;

FIG. 70 is a chart showing explanatory variables in an index formula group 12 and coefficients of those;

FIG. 71 is a chart showing correct answer rates in various cancers;

FIG. 72 is a chart showing a list of discriminant groups having the same discrimination performance as that of the index formula group 12;

FIG. 73 is a chart showing a list of discriminant groups having the same discrimination performance as that of the index formula group 12;

FIG. 74 is boxplots showing distributions of amino acid explanatory variables in various cancer patients and cancer-free subjects;

FIG. 75 is a chart showing p-values in one-way analysis of variance;

FIG. 76 is a chart plotting the third principal components and the fourth principal components obtained in principal component analysis;

FIG. 77 is a chart showing explanatory variables in an index formula group 13 and coefficients of those;

FIG. 78 is a chart showing correct answer rates in various cancers and cancer-free;

FIG. 79 is a chart showing explanatory variables in an index formula group 14 and coefficients of those;

FIG. 80 is a chart showing correct answer rates in various cancers and cancer-free;

FIG. 81 is a chart showing a list of discriminant groups having the same discrimination performance as that of the index formula group 14;

FIG. 82 is a chart showing a list of discriminant groups having the same discrimination performance as that of the index formula group 14;

FIG. 83 is a chart showing explanatory variables in an index formula group 15 and coefficients of those;

FIG. 84 is a chart showing correct answer rates in various cancers and cancer-free;

FIG. 85 is a chart showing a list of discriminant groups having the same discrimination performance as that of the index formula group 15;

FIG. 86 is a chart showing a list of discriminant groups having the same discrimination performance as that of the index formula group 15;

FIG. 87 is a chart showing explanatory variables in an index formula group 16 and coefficients of those; and

FIG. 88 is a chart showing correct answer rates in various cancers and cancer-free.

### DETAILED DESCRIPTION OF THE PREFERRED EMBODIMENTS

Hereinafter, an embodiment (first embodiment) of the method of evaluating cancer type of the present invention and an embodiment (second embodiment) of the cancer type-evaluating apparatus, the cancer type-evaluating method, the cancer type-evaluating system, the cancer type-evaluating program and the recording medium of the present invention are described in detail with reference to the drawings. The present invention is not limited to these embodiments.

### First Embodiment

### 1-1. Outline of the Invention

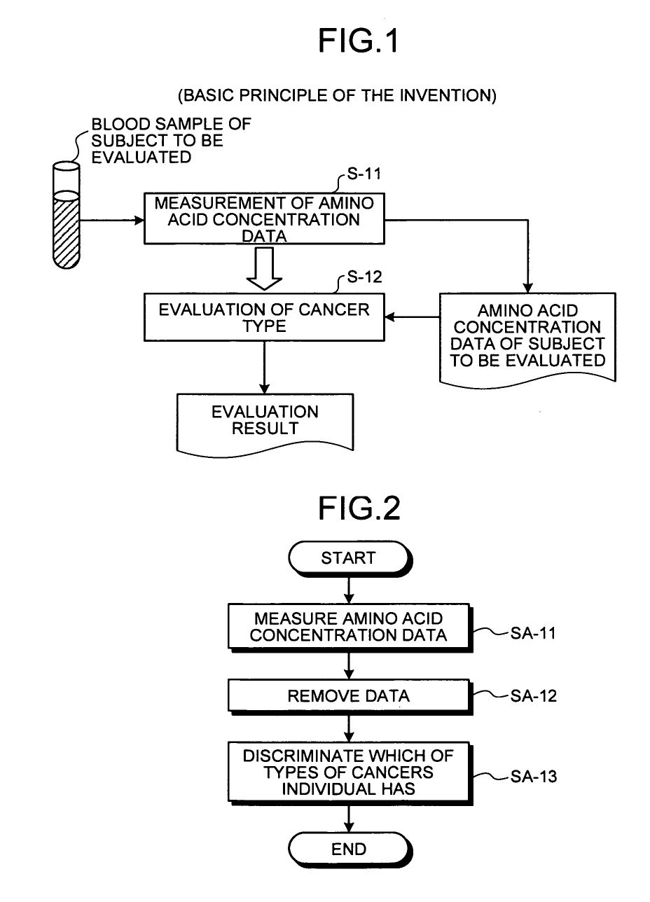

Here, an outline of the method of evaluating cancer type of the present invention will be described with reference to FIG. 1. FIG. 1 is a principle configurational diagram showing a basic principle of the present invention.

In the present invention, amino acid concentration data on a concentration value of an amino acid in blood collected from a subject (for example, an individual such as animal or human) to be evaluated is first measured (step S-11). Concentrations of amino acids in blood are analyzed in the following manner. A blood sample is collected in a heparin-treated tube, and then the blood plasma is separated by centrifugation of the collected blood sample. All blood plasma samples separated are frozen and stored at −70° C. before a measurement of amino acid concentrations. Before the measurement of amino acid concentrations, the blood plasma samples are deproteinized by adding sulfosalicylic acid to a concentration of 3%. An amino acid analyzer by high-performance liquid chromatography (HPLC) by using ninhydrin reaction in the post column is used for the measurement of amino acid concentrations. The unit of the amino acid concentration may be for example molar concentration, weight concentration, or these concentrations which are subjected to addition, subtraction, multiplication or division by an arbitrary constant.

In the present invention, a cancer type in the subject is evaluated based on the concentration value of at least one of Glu, ABA, Val, Met, Pro, Phe, Thr, Ile, Leu, and His contained in the amino acid concentration data of the subject measured in the step S-11 (step S-12).

According to the present invention described above, (i) the amino acid concentration data on the concentration value of the amino acid in blood collected from the subject is measured, and (ii) the cancer type in the subject is evaluated based on the concentration value of at least one of Glu, ABA, Val, Met, Pro, Phe, Thr, Ile, Leu, and His contained in the measured amino acid concentration data of the subject. Thus, concentrations of amino acids which among amino acids in blood, are related to states of various cancers can be utilized to bring about an effect of enabling an accurate evaluation of the cancer type. Specifically, an examinee likely to contract a plurality of cancers can be narrowed by one sample in a short time to bring about an effect of enabling a reduction of temporal, physical and financial burden of the examinee. Specifically, whether a certain sample is with cancer and where an affected area is when this is with the cancer can be evaluated accurately by concentrations of a plurality of amino acids and a discriminant group composed of one or a plurality of discriminants with the concentrations of the amino acids as the explanatory variables to bring about an effect of enabling to make the examination efficient and high accurate.

Before step S-12 is executed, data such as defective and outliers may be removed from the amino acid concentration data of the subject measured in step S-11. Thereby, the cancer type can be more accurately evaluated.

In step S-12, a cancer in the subject may be discriminated out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer) based on the concentration value of at least one of Glu, ABA, Val, Met, Pro, Phe, Thr, Ile, Leu, and His contained in the amino acid concentration data of the subject measured in step S-11. Specifically, the concentration value of at least one of Glu, ABA, Val, Met, Pro, Phe, Thr, Ile, Leu, and His may be compared with a previously established threshold (cutoff value), thereby discriminating the cancer in the subject out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer). Thus, concentrations of amino acids which among amino acids in blood, are useful for a multiple-group discrimination of cancer can be utilized to bring about an effect of enabling accurately the multiple-group discrimination of cancer.

In step S-12, (i) a discriminant value that is a value of a multivariate discriminant with a concentration of the amino acid as an explanatory variable may be calculated for each of the multivariate discriminants composing a multivariate discriminant group, based on both (a) the concentration value of at least one of Glu, ABA, Val, Met, Pro, Phe, Thr, Ile, Leu, and His contained in the amino acid concentration data of the subject measured in step S-11 and (b) the multivariate discriminant group composed of one or a plurality of the previously established multivariate discriminants containing at least one of Glu, ABA, Val, Met, Pro, Phe, Thr, Ile, Leu, and His as the explanatory variable, and (ii) the cancer type in the subject may be evaluated based on a discriminant value group composed of one or a plurality of the calculated discriminant values. Thus, a discriminant value group obtained in a multivariate discriminant group correlated significantly with states of various cancers can be utilized to bring about an effect of enabling an accurate evaluation of the cancer type. Specifically, an examinee likely to contract a plurality of cancers can be narrowed by one sample in a short time to bring about an effect of enabling a reduction of temporal, physical and financial burden of the examinee. Specifically, whether a certain sample is with cancer and where an affected area is when this is with the cancer can be evaluated accurately by concentrations of a plurality of amino acids and a discriminant group composed of one or a plurality of discriminants with the concentrations of the amino acids as the explanatory variables to bring about an effect of enabling to make the examination efficient and high accurate.

In step S-12, the cancer in the subject may be discriminated out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer) based on the calculated discriminant value group. Specifically, the discriminant value group may be compared with a previously established threshold (cutoff value), thereby discriminating the cancer in the subject out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer). Thus, a discriminant value group obtained in a multivariate discriminant group useful for a multiple-group discrimination of cancer can be utilized to bring about an effect of enabling accurately the multiple-group discrimination of cancer.

Each of the multivariate discriminants composing the multivariate discriminant group may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16. Thus, a discriminant value group obtained in a multivariate discriminant group useful particularly for a multiple-group discrimination of cancer can be utilized to bring about an effect of enabling more accurately the multiple-group discrimination of cancer.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Each multivariate discriminant composing these multivariate discriminant groups can be prepared by a method described in International Publication WO 2004/052191 that is an international application filed by the present applicant or by a method (multivariate discriminant-preparing processing described in the second embodiment described later) described in International Publication WO 2006/098192 that is an international application filed by the present applicant. Any multivariate discriminants obtained by these methods can be preferably used in the evaluation of the cancer type, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

In the fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

When the cancer type is evaluated (specifically, which of the cancers the subject has is discriminated) in the present invention, the concentrations of the other metabolites, the gene expression level, the protein expression level, the age and sex of the subject, the presence or absence of the smoking, the digitalized electrocardiogram waveform, or the like may be used in addition to the amino acid concentration. When the cancer type is evaluated (specifically, which of the cancers the subject has is discriminated) in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

### 1-2. Method of Evaluating Cancer Type in Accordance with the First Embodiment

Herein, the method of evaluating cancer type according to the first embodiment is described with reference to FIG. 2. FIG. 2 is a flowchart showing one example of the method of evaluating cancer type according to the first embodiment.

The amino acid concentration data on the concentration values of the amino acids is measured from blood collected from an individual such as animal or human (step SA-11). The measurement of the concentration values of the amino acids is conducted by the method described above.

Data such as defective and outliers is then removed from the amino acid concentration data of the individual measured in the step SA-11 (step SA-12).

Then, (I) the concentration value of at least one of Glu, ABA, Val, Met, Pro, Phe, Thr, Ile, Leu, and His contained in the amino acid concentration data of the individual from which the data such as the defective and the outliers have been removed in step SA-12 is compared with a previously established threshold (cutoff value), thereby discriminating the cancer in the individual out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer), or (II) (i) the discriminant value that is the value of the multivariate discriminant with the concentration of the amino acid as the explanatory variable is calculated for each of the multivariate discriminants composing the multivariate discriminant group, based on both (a) the concentration value of at least one of Glu, ABA, Val, Met, Pro, Phe, Thr, Ile, Leu, and His contained in the amino acid concentration data of the individual from which the data such as the defective and the outliers have been removed in step SA-12 and (b) the multivariate discriminant group composed of one or a plurality of the previously established multivariate discriminants containing at least one of Glu, ABA, Val, Met, Pro, Phe, Thr, Ile, Leu, and His as the explanatory variable, and (ii) the discriminant value group composed of one or a plurality of the calculated discriminant values is compared with a previously established threshold (cutoff value), thereby discriminating the cancer type in the individual out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer) (step SA-13).

### 1-3. Summary of the First Embodiment and Other Embodiments

In the method of evaluating cancer type as described above in detail, (1) the amino acid concentration data is measured from blood collected from the individual, (2) the data such as the defective and the outliers is removed from the measured amino acid concentration data of the individual, and (3) (I) the concentration value of at least one of Glu, ABA, Val, Met, Pro, Phe, Thr, Ile, Leu, and His contained in the amino acid concentration data of the individual from which the data such as the defective and the outliers have been removed is compared with the previously established threshold (cutoff value), thereby discriminating the cancer in the individual out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer), or (II) (i) the discriminant value that is the value of the multivariate discriminant with the concentration of the amino acid as the explanatory variable is calculated for each of the multivariate discriminants composing the multivariate discriminant group, based on both (a) the concentration value of at least one of Glu, ABA, Val, Met, Pro, Phe, Thr, Ile, Leu, and His contained in the amino acid concentration data of the individual from which the data such as the defective and the outliers have been removed and (b) the multivariate discriminant group composed of one or a plurality of the previously established multivariate discriminants containing at least one of Glu, ABA, Val, Met, Pro, Phe, Thr, Ile, Leu, and His as the explanatory variable, and (ii) the discriminant value group composed of one or a plurality of the calculated discriminant values is compared with the previously established threshold (cutoff value), thereby discriminating the cancer type in the individual out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer). Thus, concentrations of amino acids which among amino acids in blood, are useful for a multiple-group discrimination of cancer, or a discriminant value group obtained in a multivariate discriminant group useful for a multiple-group discrimination of cancer can be utilized to bring about an effect of enabling accurately the multiple-group discrimination of cancer.

In step SA-13, each of the multivariate discriminants composing the multivariate discriminant group may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16. Thus, a discriminant value group obtained in a multivariate discriminant group useful particularly for a multiple-group discrimination of cancer can be utilized to bring about an effect of enabling more accurately the multiple-group discrimination of cancer.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Each multivariate discriminant composing these multivariate discriminant groups can be prepared by a method described in International Publication WO 2004/052191 that is an international application filed by the present applicant or by a method (multivariate discriminant-preparing processing described in the second embodiment described later) described in International Publication WO 2006/098192 that is an international application filed by the present applicant. Any multivariate discriminants obtained by these methods can be preferably used in the evaluation of the cancer type, regardless of the unit of the amino acid concentration in the amino acid concentration data as input data.

### Second Embodiment

### 2-1. Outline of the Invention

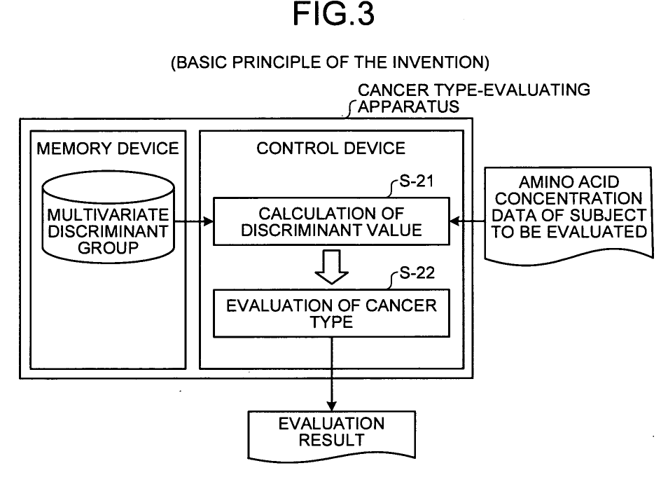

Herein, an outline of the cancer type-evaluating apparatus, the cancer type-evaluating method, the cancer type-evaluating system, the cancer type-evaluating program and the recording medium of the present invention are described in detail with reference to FIG. 3. FIG. 3 is a principle configurational diagram showing a basic principle of the present invention.

In the present invention, a discriminant value that is a value of a multivariate discriminant with a concentration of an amino acid as an explanatory variable is calculated in a control device for each of the multivariate discriminants composing a multivariate discriminant group, based on both (a) a concentration value of at least one of Glu, ABA, Val, Met, Pro, Phe, Thr, Ile, Leu, and His contained in previously obtained amino acid concentration data on the concentration value of the amino acid of a subject (for example, an individual such as animal or human) to be evaluated and (b) the multivariate discriminant group composed of one or a plurality of the multivariate discriminants stored in a memory device containing at least one of Glu, ABA, Val, Met, Pro, Phe, Thr, Ile, Leu, and His as the explanatory variable (step S-21).

In the present invention, a cancer type in the subject is evaluated in the control device based on a discriminant value group composed of one or a plurality of the discriminant values calculated in step S-21 (step S-22).

According to the present invention described above, (i) the discriminant value that is the value of the multivariate discriminant with the concentration of the amino acid as the explanatory variable is calculated for each of the multivariate discriminants composing the multivariate discriminant group, based on both (a) the concentration value of at least one of Glu, ABA, Val, Met, Pro, Phe, Thr, Ile, Leu, and His contained in the previously obtained amino acid concentration data on the concentration value of the amino acid of the subject and (b) the multivariate discriminant group composed of one or a plurality of the multivariate discriminants stored in the memory device containing at least one of Glu, ABA, Val, Met, Pro, Phe, Thr, Ile, Leu, and His as the explanatory variable, and (ii) the cancer type in the subject is evaluated based on the discriminant value group composed of one or a plurality of the calculated discriminant values. Thus, a discriminant value group obtained in a multivariate discriminant group correlated significantly with states of various cancers can be utilized to bring about an effect of enabling an accurate evaluation of the cancer type. Specifically, an examinee likely to contract a plurality of cancers can be narrowed by one sample in a short time to bring about an effect of enabling a reduction of temporal, physical and financial burden of the examinee. Specifically, whether a certain sample is with cancer and where an affected area is when this is with the cancer can be evaluated accurately by concentrations of a plurality of amino acids and a discriminant group composed of one or a plurality of discriminants with the concentrations of the amino acids as the explanatory variables to bring about an effect of enabling to make the examination efficient and high accurate.

In step S-22, a cancer in the subject may be discriminated out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer) based on the discriminant value group calculated in step S-21. Specifically, the discriminant value group may be compared with a previously established threshold (cutoff value), thereby discriminating the cancer in the subject out of at least two of colon cancer, breast cancer, prostatic cancer, thyroid cancer, lung cancer, gastric cancer, and uterine cancer (specifically, at least three of colon cancer, breast cancer, prostatic cancer, thyroid cancer, and lung cancer). Thus, a discriminant value group obtained in a multivariate discriminant group useful for a multiple-group discrimination of cancer can be utilized to bring about an effect of enabling accurately the multiple-group discrimination of cancer.

Each of the multivariate discriminants composing the multivariate discriminant group may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16. Thus, a discriminant value group obtained in a multivariate discriminant group useful particularly for a multiple-group discrimination of cancer can be utilized to bring about an effect of enabling more accurately the multiple-group discrimination of cancer.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Each multivariate discriminant composing these multivariate discriminant groups can be prepared by a method described in International Publication WO 2004/052191 that is an international application filed by the present applicant or by a method (multivariate discriminant-preparing processing described later) described in International Publication WO 2006/098192 that is an international application filed by the present applicant. Any multivariate discriminants obtained by these methods can be preferably used in the evaluation of the cancer type, regardless of the unit of the amino acid concentration in the amino acid concentration data as input data.

The multivariate discriminant refers to a form of equation used generally in multivariate analysis and includes, for example, multiple regression equation, multiple logistic regression equation, linear discriminant function, Mahalanobis' generalized distance, canonical discriminant function, support vector machine, and decision tree. The multivariate discriminant also includes an equation shown by the sum of different forms of the multivariate discriminants. In the multiple regression equation, multiple logistic regression equation and canonical discriminant function, a coefficient and constant term are added to each explanatory variable, and the coefficient and constant term in this case are preferably real numbers, more preferably values in the range of 99% confidence interval for the coefficient and constant term obtained from data for discrimination, more preferably in the range of 95% confidence interval for the coefficient and constant term obtained from data for discrimination. The value of each coefficient and the confidence interval thereof may be those multiplied by a real number, and the value of each constant term and the confidence interval thereof may be those having an arbitrary actual constant added or subtracted or those multiplied or divided by an arbitrary actual constant.

In the fractional expression, the numerator of the fractional expression is expressed by the sum of the amino acids A, B, C etc. and the denominator of the fractional expression is expressed by the sum of the amino acids a, b, c etc. The fractional expression also includes the sum of the fractional expressions α, β, γ etc. (for example, α+β) having such constitution. The fractional expression also includes divided fractional expressions. The amino acids used in the numerator or denominator may have suitable coefficients respectively. The amino acids used in the numerator or denominator may appear repeatedly. Each fractional expression may have a suitable coefficient. A value of a coefficient for each explanatory variable and a value for a constant term may be any real numbers. In combinations where explanatory variables in the numerator and explanatory variables in the denominator in the fractional expression are switched with each other, the positive (or negative) sign is generally reversed in correlation with objective explanatory variables, but because their correlation is maintained, such combinations can be assumed to be equivalent to one another in discrimination, and thus the fractional expression also includes combinations where explanatory variables in the numerator and explanatory variables in the denominator in the fractional expression are switched with each other.

When the cancer type is evaluated (specifically, which of the cancers the subject has is discriminated) in the present invention, the concentrations of the other metabolites, the gene expression level, the protein expression level, the age and sex of the subject, the presence or absence of the smoking, the digitalized electrocardiogram waveform, or the like may be used in addition to the amino acid concentration. When the cancer type is evaluated (specifically, which of the cancer the subject has is discriminated) in the present invention, the concentrations of the other metabolites, the gene expression level, the protein expression level, the age and sex of the subject, the presence or absence of the smoking, the digitalized electrocardiogram waveform, or the like may be used as the explanatory variables in the multivariate discriminant in addition to the amino acid concentration.

Here, the summary of the multivariate discriminant-preparing processing (steps 1 to 4) is described in detail. The multivariate discriminant-preparing processing is collectively executed to the data obtained by summarizing the cancers (specifically, for example, colon cancer, breast cancer, prostatic cancer, thyroid cancer, lung cancer, gastric cancer, and uterine cancer described above) being a subject when evaluating the cancer type.

First, a candidate multivariate discriminant group (e.g., y=a₁x₁+a₂x₂+ . . . +aₙxₙ, y: cancer state index data, xᵢ: amino acid concentration data, aᵢ: constant, i=1, 2, . . . , n) that is a candidate for the multivariate discriminant group is prepared in the control device based on a predetermined discriminant-preparing method from cancer state information stored in the memory device containing the amino acid concentration data and cancer state index data on an index for indicating a cancer state (step 1). Data containing defective and outliers may be removed in advance from the cancer state information.

In step 1, a plurality of the candidate multivariate discriminant groups may be prepared from the cancer state information by using a plurality of the different discriminant-preparing methods (including those for multivariate analysis such as principal component analysis, discriminant analysis, support vector machine, multiple regression analysis, logistic regression analysis, k-means method, cluster analysis, and decision tree). Specifically, a plurality of the candidate multivariate discriminant groups may be prepared simultaneously and concurrently by using a plurality of different algorithms with the cancer state information which is multivariate data composed of the amino acid concentration data and the cancer state index data obtained by analyzing blood samples from a large number of healthy subjects and cancer patients. For example, the two different candidate multivariate discriminants may be formed by performing discriminant analysis and logistic regression analysis simultaneously with the different algorithms. Alternatively, the candidate multivariate discriminant group may be formed by converting the cancer state information with the candidate multivariate discriminant group prepared by performing principal component analysis and then performing discriminant analysis of the converted cancer state information. In this way, it is possible to finally prepare the multivariate discriminant group suitable for diagnostic condition.

The candidate multivariate discriminant group prepared by principal component analysis is a linear expression consisting of amino acid explanatory variables maximizing the variance of all amino acid concentration data. The candidate multivariate discriminant group prepared by discriminant analysis is a high-powered expression (including exponential and logarithmic expressions) consisting of amino acid explanatory variables minimizing the ratio of the sum of the variances in respective groups to the variance of all amino acid concentration data. The candidate multivariate discriminant group prepared by using support vector machine is a high-powered expression (including kernel function) consisting of amino acid explanatory variables maximizing the boundary between groups. The candidate multivariate discriminant prepared by multiple regression analysis is a high-powered expression consisting of amino acid explanatory variables minimizing the sum of the distances from all amino acid concentration data. The candidate multivariate discriminant prepared by logistic regression analysis is a fraction expression having, as a component, the natural logarithm having a linear expression consisting of amino acid explanatory variables maximizing the likelihood as the exponent. The k-means method is a method of searching k pieces of neighboring amino acid concentration data in various groups, designating the group containing the greatest number of the neighboring points as its data-belonging group, and selecting the amino acid explanatory variable that makes the group to which input amino acid concentration data belong agree well with the designated group. The cluster analysis is a method of clustering (grouping) the points closest in entire amino acid concentration data. The decision tree is a method of ordering amino acid explanatory variables and predicting the group of amino acid concentration data from the pattern possibly held by the higher-ordered amino acid explanatory variable.

Returning to the description of the multivariate discriminant-preparing processing, the candidate multivariate discriminant group prepared in the step 1 is verified (mutually verified) in the control device based on a particular verifying method (step 2). The verification of the candidate multivariate discriminant group is performed on each other to each candidate multivariate discriminant group prepared in the step 1.

In the step 2, at least one of discrimination rate, sensitivity, specificity, information criterion, and the like of the candidate multivariate discriminant group may be verified by at least one of the bootstrap method, holdout method, leave-one-out method, and the like. In this way, it is possible to prepare the candidate multivariate discriminant group higher in predictability or reliability, by taking the cancer state information and the diagnostic condition into consideration.

The discrimination rate is the rate of the cancer state judged correct according to the present invention in all input data. The sensitivity is the rate of the cancer states judged correct according to the present invention in the cancer states declared cancer in the input data. The specificity is the rate of the cancer states judged correct according to the present invention in the cancer states declared healthy in the input data. The information criterion is the sum of the number of the amino acid explanatory variables in the candidate multivariate discriminant group prepared in the step 1 and the difference in number between the cancer states evaluated according to the present invention and those declared in input data. The predictability is the average of the discrimination rate, sensitivity, or specificity obtained by repeating verification of the candidate multivariate discriminant group. Alternatively, the reliability is the variance of the discrimination rate, sensitivity, or specificity obtained by repeating verification of the candidate multivariate discriminant group.

Returning to the description of the multivariate discriminant-preparing processing, a combination of the amino acid concentration data contained in the cancer state information used in preparing the candidate multivariate discriminant group is selected by selecting the explanatory variable of the candidate multivariate discriminant group in the control device based on a predetermined explanatory variable-selecting method from the verification result obtained in the step 2 (step 3). The selection of the amino acid explanatory variable is performed on each candidate multivariate discriminant group prepared in the step 1. In this way, it is possible to select the amino acid explanatory variable of the candidate multivariate discriminant group properly. The step 1 is executed once again by using the cancer state information including the amino acid concentration data selected in the step 3.

In the step 3, the amino acid explanatory variable of the candidate multivariate discriminant group may be selected based on at least one of the stepwise method, best path method, local search method, and genetic algorithm from the verification result obtained in the step 2.

The best path method is a method of selecting an amino acid explanatory variable by optimizing an evaluation index of the candidate multivariate discriminant group while eliminating the amino acid explanatory variables contained in the candidate multivariate discriminant group one by one.

Returning to the description of the multivariate discriminant-preparing processing, the steps 1, 2 and 3 are repeatedly performed in the control device, and based on verification results thus accumulated, the candidate multivariate discriminant group used as the multivariate discriminant group is selected from a plurality of the candidate multivariate discriminant groups, thereby preparing the multivariate discriminant group (step 4). In the selection of the candidate multivariate discriminant group, there are cases where the optimum multivariate discriminant group is selected from the candidate multivariate discriminant groups prepared in the same discriminant-preparing method or the optimum multivariate discriminant group is selected from all candidate multivariate discriminant groups.

As described above, in the multivariate discriminant-preparing processing, the processing for the preparation of the candidate multivariate discriminant groups, the verification of the candidate multivariate discriminant groups, and the selection of the explanatory variables in the candidate multivariate discriminant groups are performed based on the cancer state information in a series of operations in a systematized manner, whereby the multivariate discriminant most appropriate for evaluating each cancer state can be prepared to enable to obtain the multivariate discriminant group most appropriate for evaluating the cancer type (specifically, the multivariate discriminant group for the multiple-group discrimination of cancer).

### 2-2. System Configuration

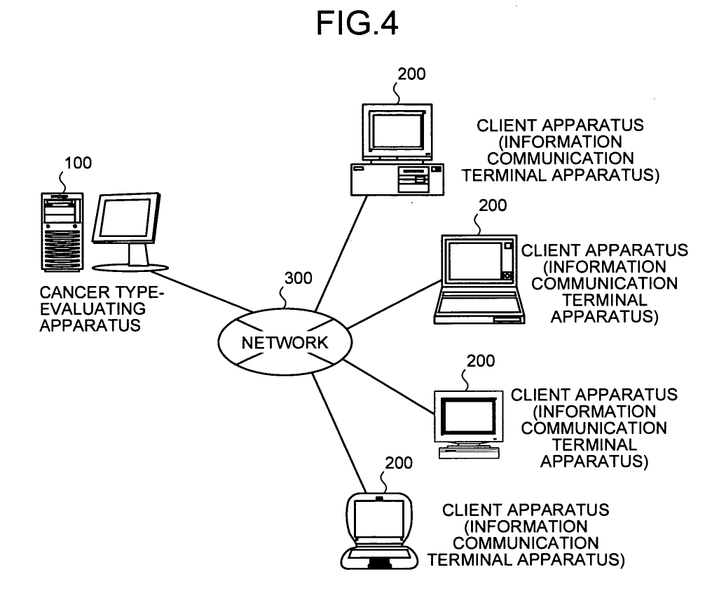

Hereinafter, the configuration of the cancer type-evaluating system according to the second embodiment (hereinafter referred to sometimes as the present system) will be described with reference to FIGS. 4 to 20. This system is merely one example, and the present invention is not limited thereto.

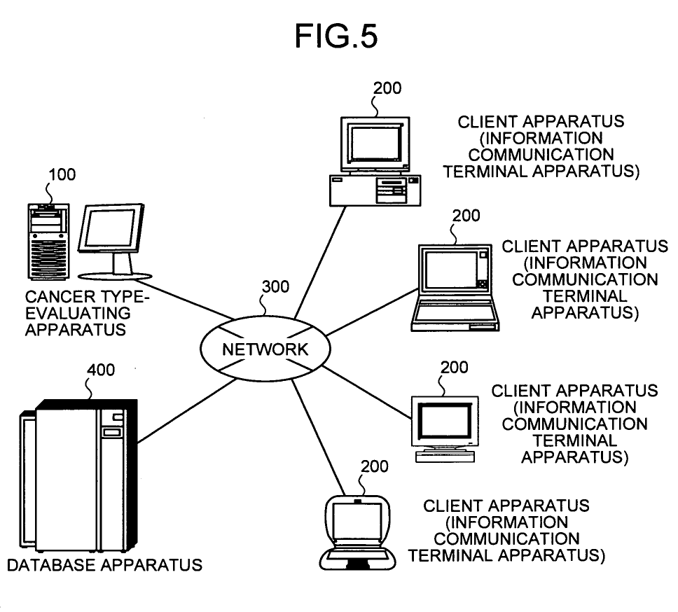

First, an entire configuration of the present system will be described with reference to FIGS. 4 and 5. FIG. 4 is a diagram showing an example of the entire configuration of the present system. FIG. 5 is a diagram showing another example of the entire configuration of the present system. As shown in FIG. 4, the present system is constituted in which the cancer type-evaluating apparatus 100 that evaluates the cancer type in the subject, and the client apparatus 200 (corresponding to the information communication terminal apparatus of the present invention) that provides the amino acid concentration data of the subject on the concentration values of the amino acids, are communicatively connected to each other via a network 300.

In the present system as shown in FIG. 5, in addition to the cancer type-evaluating apparatus 100 and the client apparatus 200, the database apparatus 400 storing, for example, the cancer state information used in preparing the multivariate discriminant and the multivariate discriminant used in evaluating the cancer state in the cancer type-evaluating apparatus 100, may be communicatively connected via the network 300. In this configuration, the information on the cancer state etc. are provided via the network 300 from the cancer type-evaluating apparatus 100 to the client apparatuses 200 and the database apparatus 400, or from the client apparatuses 200 and the database apparatus 400 to the cancer type-evaluating apparatus 100. The “information on the cancer state” is information on the measured values of particular items of the cancer state of organisms including human. The information on the cancer state is generated in the cancer type-evaluating apparatus 100, client apparatus 200, or other apparatuses (e.g., various measuring apparatuses) and stored mainly in the database apparatus 400.

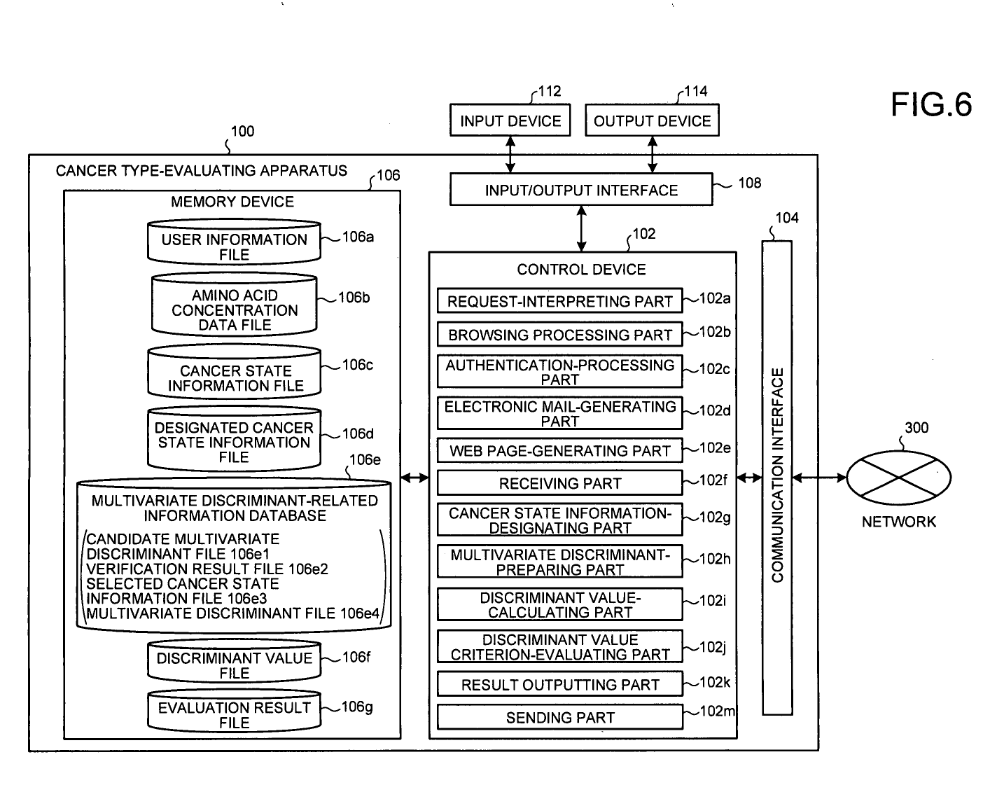

Now, the configuration of the cancer type-evaluating apparatus 100 in the present system will be described with reference to FIGS. 6 to 18. FIG. 6 is a block diagram showing an example of the configuration of the cancer type-evaluating apparatus 100 in the present system, showing conceptually only the region relevant to the present invention.

The cancer type-evaluating apparatus 100 includes (a) a control device 102, such as CPU (Central Processing Unit), that integrally controls the cancer type-evaluating apparatus 100, (b) a communication interface 104 that connects the cancer type-evaluating apparatus 100 to the network 300 communicatively via communication apparatuses such as a router and wired or wireless communication lines such as a private line, (c) a memory device 106 that stores various databases, tables, files and others, and (d) an input/output interface 108 connected to an input device 112 and an output device 114, and these parts are connected to each other communicatively via any communication channel. The cancer type-evaluating apparatus 100 may be present together with various analyzers (e.g., amino acid analyzer) in a same housing. A typical configuration of disintegration/integration of the cancer type-evaluating apparatus 100 is not limited to that shown in the figure, and all or a part of it may be disintegrated or integrated functionally or physically in any unit, for example, according to various loads applied. For example, a part of the processing may be performed via CGI (Common Gateway Interface).

The memory device 106 is a storage means, and examples thereof include memory apparatuses such as RAM (Random Access Memory) and ROM (Read Only Memory), fixed disk drives such as a hard disk, a flexible disk, an optical disk, and the like. The memory device 106 stores computer programs giving instructions to the CPU for various processings, together with OS (Operating System). As shown in the figure, the memory device 106 stores the user information file 106a, the amino acid concentration data file 106b, the cancer state information file 106c, the designated cancer state information file 106d, a multivariate discriminant-related information database 106e, the discriminant value file 106f and the evaluation result file 106g.

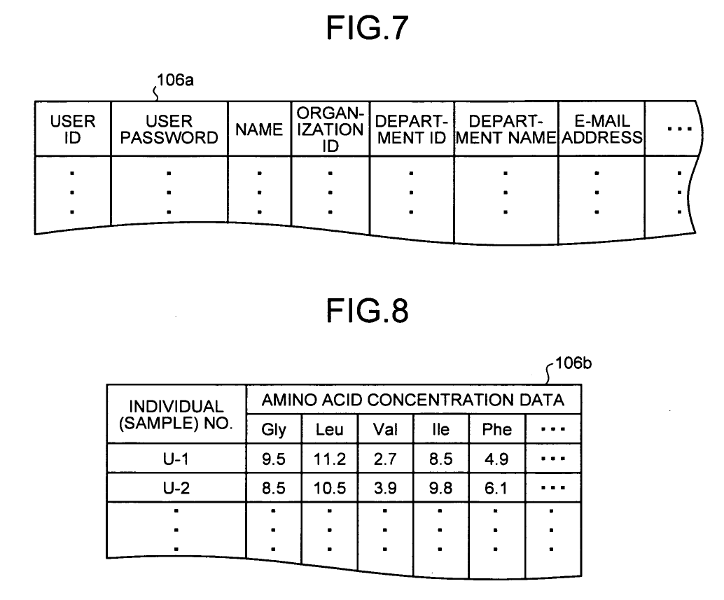

The user information file 106a stores user information on users. FIG. 7 is a chart showing an example of information stored in the user information file 106a. As shown in FIG. 7, the information stored in the user information file 106a includes user ID (identification) for identifying a user uniquely, user password for authentication of the user, user name, organization ID for uniquely identifying an organization of the user, department ID for uniquely identifying a department of the user organization, department name, and electronic mail address of the user that are correlated to one another.

Returning to FIG. 6, the amino acid concentration data file 106b stores the amino acid concentration data on the concentration values of the amino acids. FIG. 8 is a chart showing an example of information stored in the amino acid concentration data file 106b. As shown in FIG. 8, the information stored in the amino acid concentration data file 106b includes individual number for uniquely identifying an individual (sample) as a subject to be evaluated and amino acid concentration data that are correlated to one another. In FIG. 8, the amino acid concentration data is assumed to be numerical values, i.e., on a continuous scale, but the amino acid concentration data may be expressed on a nominal scale or an ordinal scale. In the case of the nominal or ordinal scale, any number may be allocated to each state for analysis. The amino acid concentration data may be combined with other biological information (e.g., the concentrations of metabolites other than the amino acids, the gene expression level, the protein expression level, the age and sex of the subject, the presence or absence of the smoking, and the digitalized electrocardiogram waveform).

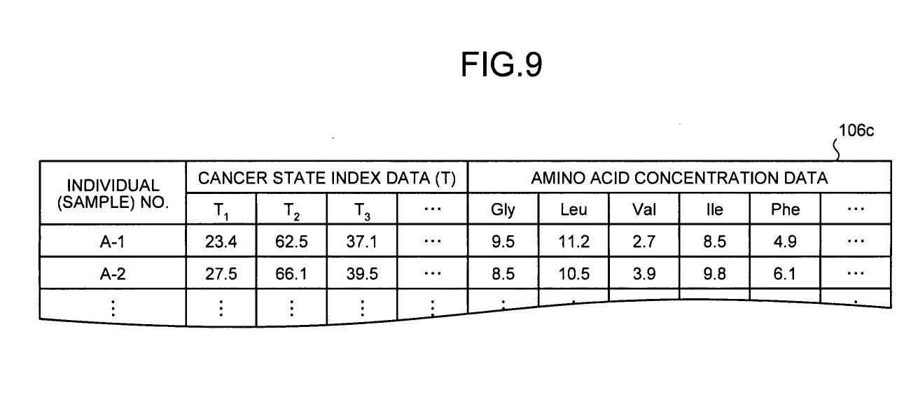

Returning to FIG. 6, the cancer state information file 106c stores the cancer state information used in preparing the multivariate discriminant. FIG. 9 is a chart showing an example of information stored in the cancer state information file 106c. As shown in FIG. 9, the information stored in the cancer state information file 106c includes individual (sample) number, cancer state index data (T) corresponding to a cancer state index (index T₁, index T₂, index T₃ . . . ), and amino acid concentration data that are correlated to one another. In FIG. 9, the cancer state index data and the amino acid concentration data are assumed to be numerical values, i.e., on a continuous scale, but the cancer state index data and the amino acid concentration data may be expressed on a nominal scale or an ordinal scale. In the case of the nominal or ordinal scale, any number may be allocated to each state for analysis. The cancer state index data is a single known condition index serving as a marker of the cancer state, and numerical data may be used.

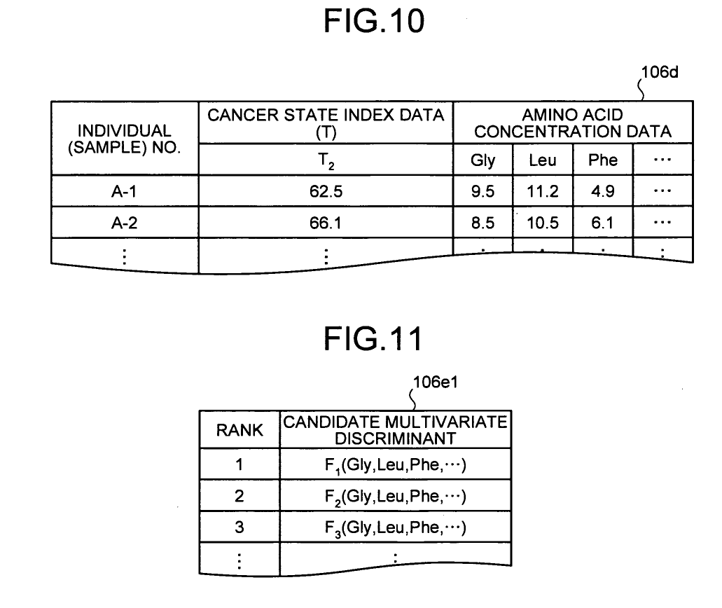

Returning to FIG. 6, the designated cancer state information file 106d stores the cancer state information designated in a cancer state information-designating part 102g described below. FIG. 10 is a chart showing an example of information stored in the designated cancer state information file 106d. As shown in FIG. 10, the information stored in the designated cancer state information file 106d includes individual number, designated cancer state index data, and designated amino acid concentration data that are correlated to one another.

Returning to FIG. 6, the multivariate discriminant-related information database 106e is composed of (i) the candidate multivariate discriminant file 106e1 storing the candidate multivariate discriminant group prepared in a candidate multivariate discriminant-preparing part 102h1 described below, (ii) the verification result file 106e2 storing the verification results obtained in a candidate multivariate discriminant-verifying part 102h2 described below, (iii) the selected cancer state information file 106e3 storing the cancer state information containing the combination of the amino acid concentration data selected in an explanatory variable-selecting part 102h3 described below, and (iv) the multivariate discriminant file 106e4 storing the multivariate discriminant group prepared in the multivariate discriminant-preparing part 102h described below.

The candidate multivariate discriminant file 106e1 stores the candidate multivariate discriminant groups prepared in the candidate multivariate discriminant-preparing part 102h1 described below. FIG. 11 is a chart showing an example of information stored in the candidate multivariate discriminant file 106e1. As shown in FIG. 11, the information stored in the candidate multivariate discriminant file 106e1 includes rank, and candidate multivariate discriminant (e.g., F₁ (Gly, Leu, Phe, . . . ), F₂ (Gly, Leu, Phe, . . . ), or F₃ (Gly, Leu, Phe, . . . ) in FIG. 11) that are correlated to each other.

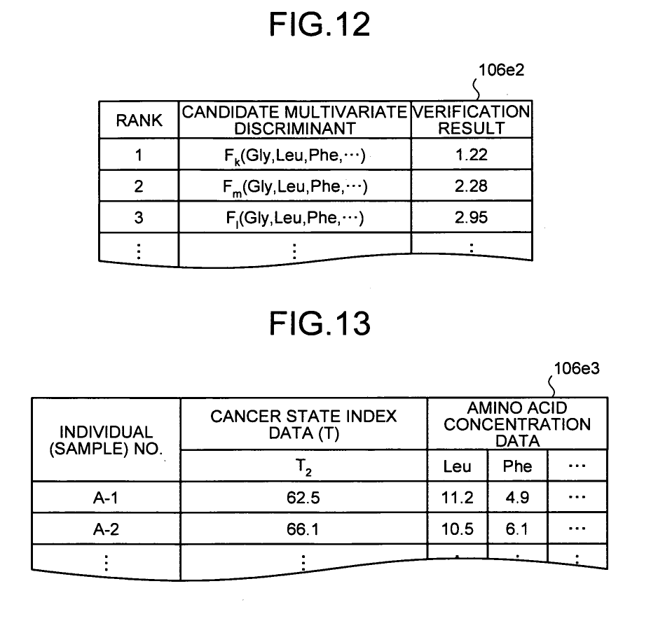

Returning to FIG. 6, the verification result file 106e2 stores the verification results obtained in the candidate multivariate discriminant-verifying part 102h2 described below. FIG. 12 is a chart showing an example of information stored in the verification result file 106e2. As shown in FIG. 12, the information stored in the verification result file 106e2 includes rank, candidate multivariate discriminant (e.g., Fₖ (Gly, Leu, Phe, . . . ), Fₘ (Gly, Leu, Phe, . . . ), Fₗ (Gly, Leu, Phe, . . . ) in FIG. 12), and verification result of each candidate multivariate discriminant (e.g., evaluation value of each candidate multivariate discriminant) that are correlated to one another.

Returning to FIG. 6, the selected cancer state information file 106e3 stores the cancer state information including the combination of the amino acid concentration data corresponding to the explanatory variables selected in the explanatory variable-selecting part 102h3 described below. FIG. 13 is a chart showing an example of information stored in the selected cancer state information file 106e3. As shown in FIG. 13, the information stored in the selected cancer state information file 106e3 includes individual number, cancer state index data designated in the cancer state information-designating part 102g described below, and amino acid concentration data selected in the explanatory variable-selecting part 102h3 described below that are correlated to one another.

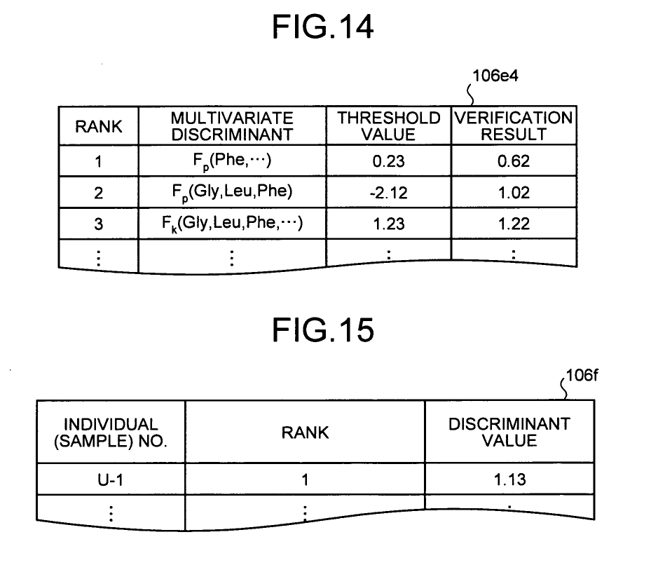

Returning to FIG. 6, the multivariate discriminant file 106e4 stores the multivariate discriminant groups prepared in the multivariate discriminant-preparing part 102h described below. FIG. 14 is a chart showing an example of information stored in the multivariate discriminant file 106e4. As shown in FIG. 14, the information stored in the multivariate discriminant file 106e4 includes rank, multivariate discriminant (e.g., Fₚ (Phe, . . . ), Fₚ (Gly, Leu, Phe), Fₖ (Gly, Leu, Phe, . . . ) in FIG. 14), a threshold corresponding to each discriminant-preparing method, and verification result of each multivariate discriminant (e.g., evaluation value of each multivariate discriminant) that are correlated to one another.

Returning to FIG. 6, the discriminant value file 106f stores the discriminant value calculated in a discriminant value-calculating part 102i described below. FIG. 15 is a chart showing an example of information stored in the discriminant value file 106f. As shown in FIG. 15, the information stored in the discriminant value file 106f includes individual number for uniquely identifying the individual (sample) as the subject, rank (number for uniquely identifying the multivariate discriminant), and discriminant value that are correlated to one another.

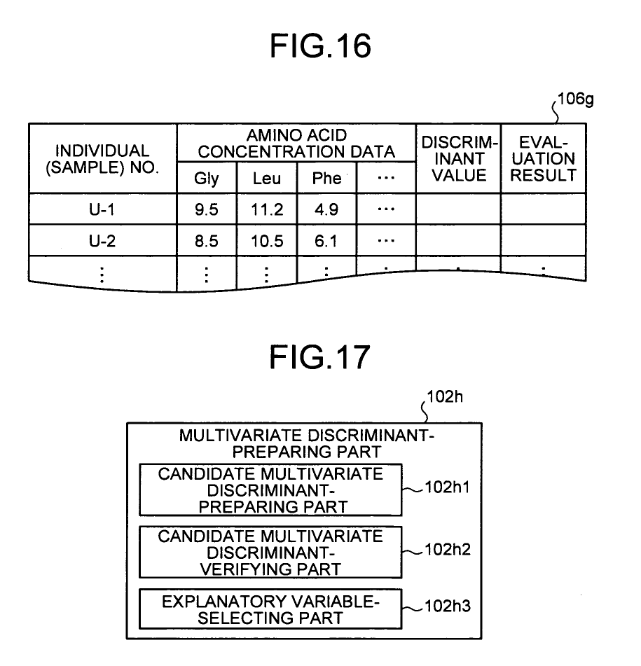

Returning to FIG. 6, the evaluation result file 106g stores the evaluation results obtained in the discriminant value criterion-evaluating part 102j described below (specifically the discrimination results obtained in a discriminant value criterion-discriminating part 102j1 described below). FIG. 16 is a chart showing an example of information stored in the evaluation result file 106g. The information stored in the evaluation result file 106g includes individual number for uniquely identifying the individual (sample) as the subject, previously obtained amino acid concentration data of the subject, one or a plurality of the discriminant values calculated by each multivariate discriminant, and evaluation result on the cancer type (specifically, discrimination result on which of the cancers the individual has) that are correlated to one another.

Returning to FIG. 6, the memory device 106 stores various Web data for providing the client apparatuses 200 with web site information, CGI programs, and others as information other than the information described above. The Web data include data for displaying the Web pages described below and others, and the data are generated as, for example, a HTML (HyperText Markup Language) or XML (Extensible Markup Language) text file. Files for components and files for operation for generation of the Web data, and other temporary files, and the like are also stored in the memory device 106. In addition, the memory device 106 may store as needed sound files of sounds for transmission to the client apparatuses 200 in WAVE format or AIFF (Audio Interchange File Format) format and image files of still images or motion pictures in JPEG (Joint Photographic Experts Group) format or MPEG2 (Moving Picture Experts Group phase 2) format.

The communication interface 104 allows communication between the cancer type-evaluating apparatus 100 and the network 300 (or communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as a monitor 114). A keyboard, a mouse, a microphone, or a monitor functioning as a pointing device together with a mouse may be used as the input device 112.

The control device 102 has an internal memory storing control programs such as OS (Operating System), programs for various processing procedures, and other needed data, and performs various information processings according to these programs. As shown in the figure, the control device 102 includes mainly a request-interpreting part 102a, a browsing processing part 102b, an authentication-processing part 102c, an electronic mail-generating part 102d, a Web page-generating part 102e, a receiving part 102f, the cancer state information-designating part 102g, the multivariate discriminant-preparing part 102h, the discriminant value-calculating part 102i, the discriminant value criterion-evaluating part 102j, a result outputting part 102k and a sending part 102m. The control device 102 performs data processings such as removal of data including defective, removal of data including many outliers, and removal of explanatory variables for the defective-including data in the cancer state information transmitted from the database apparatus 400 and in the amino acid concentration data transmitted from the client apparatus 200.

The request-interpreting part 102a interprets the requests transmitted from the client apparatus 200 or the database apparatus 400 and sends the requests to other parts in the control device 102 according to results of interpreting the requests. Upon receiving browsing requests for various screens transmitted from the client apparatus 200, the browsing processing part 102b generates and transmits web data for these screens. Upon receiving authentication requests transmitted from the client apparatus 200 or the database apparatus 400, the authentication-processing part 102c performs authentication. The electronic mail-generating part 102d generates electronic mails including various kinds of information. The Web page-generating part 102e generates Web pages for users to browse with the client apparatus 200.

The receiving part 102f receives, via the network 300, information (specifically, the amino acid concentration data, the cancer state information, the multivariate discriminant group etc.) transmitted from the client apparatus 200 and the database apparatus 400. The cancer state information-designating part 102g designates objective cancer state index data and objective amino acid concentration data in preparing the multivariate discriminant group.

The multivariate discriminant-preparing part 102h generates the multivariate discriminant groups based on the cancer state information received in the receiving part 102f and the cancer state information designated in the cancer state information-designating part 102g. Specifically, the multivariate discriminant-preparing part 102h generates the multivariate discriminant group by selecting the candidate multivariate discriminant group used as the multivariate discriminant group from a plurality of the candidate multivariate discriminant groups, based on verification results accumulated by repeating processings in the candidate multivariate discriminant-preparing part 102h1, the candidate multivariate discriminant-verifying part 102h2, and the explanatory variable-selecting part 102h3 from the cancer state information.

If the multivariate discriminant groups are stored previously in a predetermined region of the memory device 106, the multivariate discriminant-preparing part 102h may generate the multivariate discriminant group by selecting the desired multivariate discriminant group out of the memory device 106. Alternatively, the multivariate discriminant-preparing part 102h may generate the multivariate discriminant group by selecting and downloading the desired multivariate discriminant group from the multivariate discriminant groups previously stored in another computer apparatus (e.g., the database apparatus 400).

Hereinafter, a configuration of the multivariate discriminant-preparing part 102h will be described with reference to FIG. 17. FIG. 17 is a block diagram showing the configuration of the multivariate discriminant-preparing part 102h, and only a part in the configuration related to the present invention is shown conceptually. The multivariate discriminant-preparing part 102h has the candidate multivariate discriminant-preparing part 102h1, the candidate multivariate discriminant-verifying part 102h2, and the explanatory variable-selecting part 102h3, additionally. The candidate multivariate discriminant-preparing part 102h1 generates the candidate multivariate discriminant group that is a candidate of the multivariate discriminant group, from the cancer state information based on a predetermined discriminant-preparing method. The candidate multivariate discriminant-preparing part 102h1 may generate a plurality of the candidate multivariate discriminant groups from the cancer state information, by using a plurality of the different discriminant-preparing methods. The candidate multivariate discriminant-verifying part 102h2 verifies the candidate multivariate discriminant group prepared in the candidate multivariate discriminant-preparing part 102h1 based on a particular verifying method. The candidate multivariate discriminant-verifying part 102h2 may verify at least one of the discrimination rate, sensitivity, specificity, and information criterion of the candidate multivariate discriminant groups based on at least one of the bootstrap method, holdout method, and leave-one-out method. The explanatory variable-selecting part 102h3 selects the combination of the amino acid concentration data contained in the cancer state information used in preparing the candidate multivariate discriminant group, by selecting the explanatory variables of the candidate multivariate discriminant group based on a particular explanatory variable-selecting method from the verification results obtained in the candidate multivariate discriminant-verifying part 102h2. The explanatory variable-selecting part 102h3 may select the explanatory variables of the candidate multivariate discriminant group based on at least one of the stepwise method, best path method, local search method, and genetic algorithm from the verification results.

Returning to FIG. 6, the discriminant value-calculating part 102i calculates the discriminant value that is the value of the multivariate discriminant, for each of the multivariate discriminants composing the multivariate discriminant group, based on both (a) the concentration value of at least one of Glu, ABA, Val, Met, Pro, Phe, Thr, Ile, Leu, and His contained in the amino acid concentration data of the subject received in the receiving part 102f and (b) the multivariate discriminant group composed of one or a plurality of the multivariate discriminants containing at least one of Glu, ABA, Val, Met, Pro, Phe, Thr, Ile, Leu, and His as the explanatory variable prepared in the multivariate discriminant-preparing part 102h.

Each of the multivariate discriminants composing the multivariate discriminant group may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

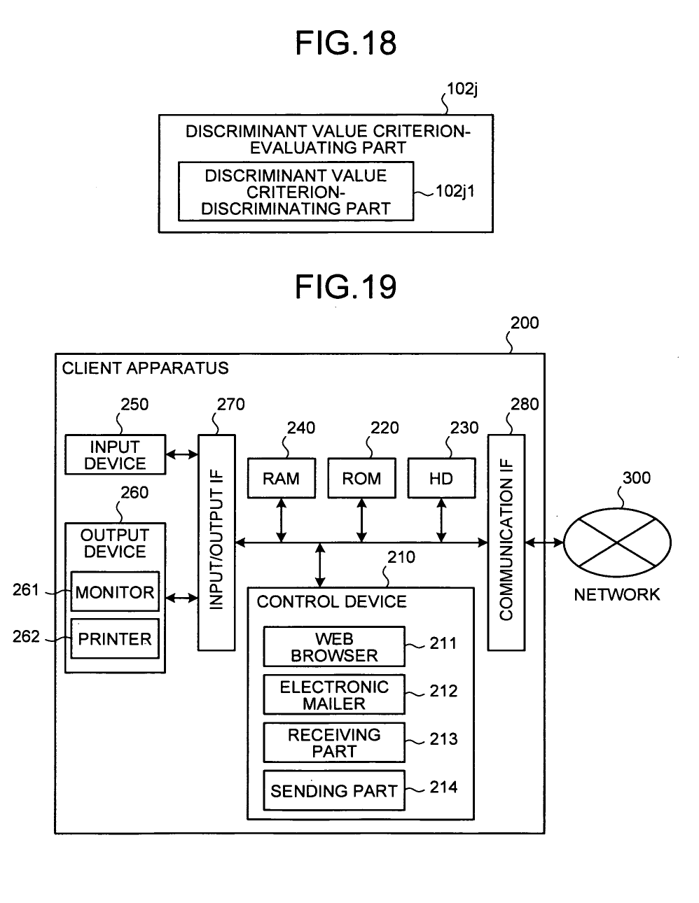

The discriminant value criterion-evaluating part 102j evaluates the cancer type in the subject based on the discriminant value group composed of one or a plurality of the discriminant values calculated in the discriminant value-calculating part 102i. The discriminant value criterion-evaluating part 102j further includes the discriminant value criterion-discriminating part 102j1. Now, the configuration of the discriminant value criterion-evaluating part 102j will be described with reference to FIG. 18. FIG. 18 is a block diagram showing the configuration of the discriminant value criterion-evaluating part 102j, and only a part in the configuration related to the present invention is shown conceptually. The discriminant value criterion-discriminating part 102j1 discriminates the cancer in the subject out of the previously established types of cancers (specifically, at least two cancers of colon cancer, breast cancer, prostatic cancer, thyroid cancer, lung cancer, gastric cancer, and uterine cancer (more specifically, at least three cancers of colon cancer, breast cancer, prostatic cancer, thyroid cancer, and lung cancer)) based on the discriminant value group. Specifically, the discriminant value criterion-discriminating part 102j1 compares the discriminant value group with a predetermined threshold value (cutoff value), thereby discriminating the cancer in the subject out of the previously established types of cancers (specifically, at least two cancers of colon cancer, breast cancer, prostatic cancer, thyroid cancer, lung cancer, gastric cancer, and uterine cancer (more specifically, at least three cancers of colon cancer, breast cancer, prostatic cancer, thyroid cancer, and lung cancer)).

Returning to FIG. 6, the result outputting part 102k outputs, into the output device 114, the processing results in each processing part in the control device 102 (the evaluation results obtained in the discriminant value criterion-evaluating part 102j (specifically, the discrimination results obtained in the discriminant value criterion-discriminating part 102j1)) etc.

The sending part 102m transmits the evaluation results to the client apparatus 200 that is a sender of the amino acid concentration data of the subject, and transmits the multivariate discriminant prepared in the cancer type-evaluating apparatus 100 and the evaluation results to the database apparatus 400.

Hereinafter, a configuration of the client apparatus 200 in the present system will be described with reference to FIG. 19. FIG. 19 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a Web browser 211, an electronic mailer 212, a receiving part 213, and a sending part 214. The Web browser 211 performs browsing processings of interpreting Web data and displaying the interpreted Web data on a monitor 261 described below. The Web browser 211 may have various plug-in softwares, such as stream player, having functions to receive, display and feedback streaming screen images. The electronic mailer 212 sends and receives electronic mails using a particular protocol (e.g., SMTP (Simple Mail Transfer Protocol) or POP3 (Post Office Protocol version 3)). The receiving part 213 receives various kinds of information, such as the evaluation results transmitted from the cancer type-evaluating apparatus 100, via the communication IF 280. The sending part 214 sends various kinds of information such as the amino acid concentration data of the subject, via the communication IF 280, to the cancer type-evaluating apparatus 100.

The input device 250 is for example a keyboard, a mouse or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatuses 200 are connected to the network 300 via a communication apparatus such as a modem, TA (Terminal Adapter) or a router, and a telephone line, or a private line. In this way, the client apparatuses 200 can access to the cancer type-evaluating apparatus 100 by using a particular protocol.

The client apparatus 200 may be realized by installing softwares (including programs, data and others) for a Web data-browsing function and an electronic mail-processing function to an information processing apparatus (for example, an information processing terminal such as a known personal computer, a workstation, a family computer, Internet TV (Television), PHS (Personal Handyphone System) terminal, a mobile phone terminal, a mobile unit communication terminal or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

Hereinafter, the network 300 in the present system will be described with reference to FIGS. 4 and 5. The network 300 has a function to connect the cancer type-evaluating apparatus 100, the client apparatuses 200, and the database apparatus 400 mutually, communicatively to one another, and is for example the Internet, an intranet, or LAN (Local Area Network (both wired/wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

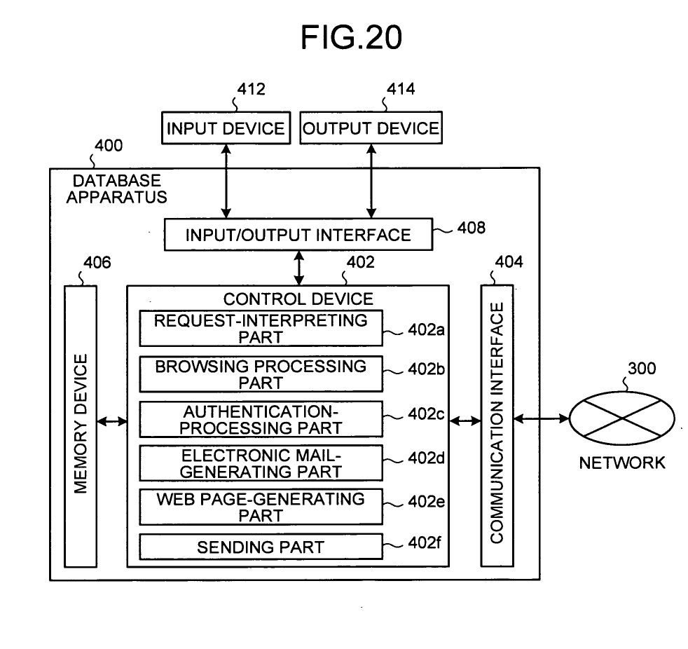

Hereinafter, the configuration of the database apparatus 400 in the present system will be described with reference to FIG. 20. FIG. 20 is a block diagram showing an example of the configuration of the database apparatus 400 in the present system, showing conceptually only the region relevant to the present invention.

The database apparatus 400 has functions to store, for example, the cancer state information used in preparing the multivariate discriminant groups in the cancer type-evaluating apparatus 100 or in the database apparatus 400, the multivariate discriminant groups prepared in the cancer type-evaluating apparatus 100, and the evaluation results obtained in the cancer type-evaluating apparatus 100. As shown in FIG. 20, the database apparatus 400 includes (a) a control device 402, such as CPU, which integrally controls the entire database apparatus 400, (b) a communication interface 404 connecting the database apparatus to the network 300 communicatively via a communication apparatus such as a router and via wired or wireless communication circuits such as a private line, (c) a memory device 406 storing various databases, tables and files (for example, files for Web pages), and (d) an input/output interface 408 connected to an input device 412 and an output device 414, and these parts are connected communicatively to each other via any communication channel.

The memory device 406 is a storage means, and may be, for example, memory apparatus such as RAM or ROM, a fixed disk drive such as a hard disk, a flexible disk, an optical disk, and the like. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414 (hereinafter, the output device 414 may be described as a monitor 414). A keyboard, a mouse, a microphone, or a monitor functioning as a pointing device together with a mouse may be used as the input device 412.

The control device 402 has an internal memory storing control programs such as OS (Operating System), programs for various processing procedures, and other needed data, and performs various information processings according to these programs. As shown in the figure, the control device 402 includes mainly a request-interpreting part 402a, a browsing processing part 402b, an authentication-processing part 402c, an electronic mail-generating part 402d, a Web page-generating part 402e, and a sending part 402f.

The request-interpreting part 402a interprets the requests transmitted from the cancer type-evaluating apparatus 100 and sends the requests to other parts in the control device 402 according to results of interpreting the requests. Upon receiving browsing requests for various screens transmitted from the cancer type-evaluating apparatus 100, the browsing processing part 402b generates and transmits web data for these screens. Upon receiving authentication requests transmitted from the cancer type-evaluating apparatus 100, the authentication-processing part 402c performs authentication. The electronic mail-generating part 402d generates electronic mails including various kinds of information. The Web page-generating part 402e generates Web pages for users to browse with the client apparatus 200. The sending part 402f transmits various kinds of information such as the cancer state information and the multivariate discriminant groups to the cancer type-evaluating apparatus 100.

### 2-3. Processing in the Present System

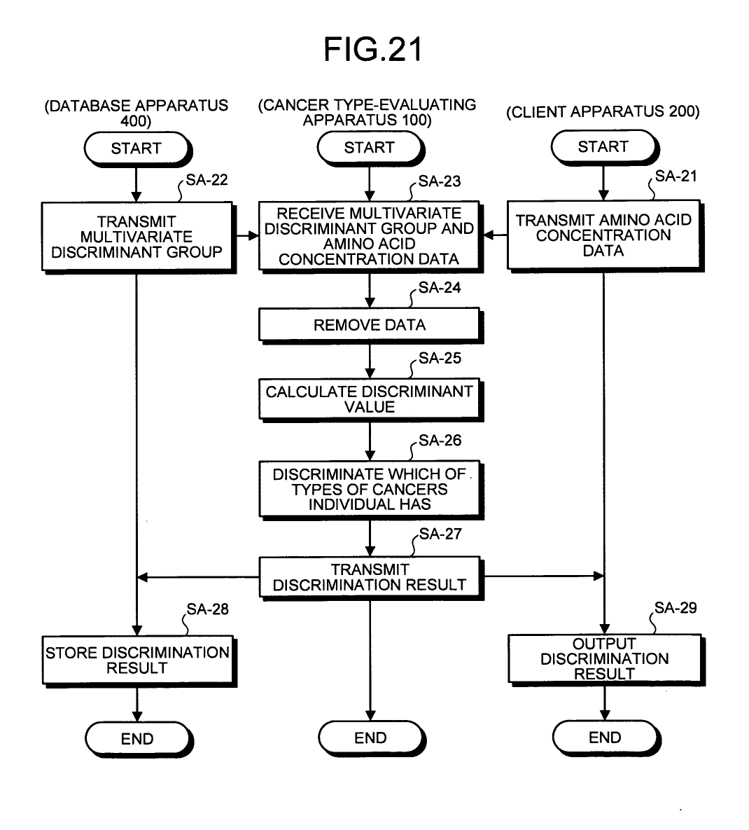

Here, an example of a cancer type evaluation service processing performed in the present system constituted as described above will be described with reference to FIG. 21. FIG. 21 is a flowchart showing the example of the cancer type evaluation service processing.

The amino acid concentration data used in the present processing is data concerning the concentration values of amino acids obtained by analyzing blood previously collected from an individual. Hereinafter, the method of analyzing blood amino acid will be described briefly. First, a blood sample is collected in a heparin-treated tube, and then the blood plasma is separated by centrifugation of the tube. All blood plasma samples separated are frozen and stored at −70° C. before a measurement of an amino acid concentration. Before the measurement of the amino acid concentration, the blood plasma samples are deproteinized by adding sulfosalicylic acid to a concentration of 3%. An amino acid analyzer by high-performance liquid chromatography (HPLC) by using ninhydrin reaction in the post column is used for the measurement of the amino acid concentration.

First, the client apparatus 200 accesses the cancer type-evaluating apparatus 100 when the user specifies the Web site address (such as URL) provided from the cancer type-evaluating apparatus 100, via the input device 250 on the screen displaying the Web browser 211. Specifically, when the user instructs update of the Web browser 211 screen on the client apparatus 200, the Web browser 211 sends the Web site address provided from the cancer type-evaluating apparatus 100 by a particular protocol to the cancer type-evaluating apparatus 100, thereby transmitting requests demanding a transmission of Web page corresponding to an amino acid concentration data transmission screen to the cancer type-evaluating apparatus 100 based on a routing of the address.

Then, upon receipt of the request transmitted from the client apparatus 200, the request-interpreting part 102a in the cancer type-evaluating apparatus 100 analyzes the transmitted requests and sends the requests to other parts in the control device 102 according to analytical results. Specifically, when the transmitted requests are requests to send the Web page corresponding to the amino acid concentration data transmission screen, mainly the browsing processing part 102b in the cancer type-evaluating apparatus 100 obtains the Web data for display of the Web page stored in a predetermined region of the memory device 106 and sends the obtained Web data to the client apparatus 200. More specifically, upon receiving the requests to transmit the Web page corresponding to the amino acid concentration data transmission screen by the user, the control device 102 in the cancer type-evaluating apparatus 100 demands inputs of user ID and user password from the user. If the user ID and password are input, the authentication-processing part 102c in the cancer type-evaluating apparatus 100 examines the input user ID and password by comparing them with the user ID and user password stored in the user information file 106a for authentication. Only when the user is authenticated, the browsing processing part 102b in the cancer type-evaluating apparatus 100 sends the Web data for displaying the Web page corresponding to the amino acid concentration data transmission screen to the client apparatus 200. The client apparatus 200 is identified with the IP (Internet Protocol) address transmitted from the client apparatus 200 together with the transmission requests.

Then, the client apparatus 200 receives, in the receiving part 213, the Web data (for displaying the Web page corresponding to the amino acid concentration data transmission screen) transmitted from the cancer type-evaluating apparatus 100, interprets the received Web data with the Web browser 211, and displays the amino acid concentration data transmission screen on the monitor 261.

When the user inputs and selects, via the input device 250, for example the amino acid concentration data of the individual on the amino acid concentration data transmission screen displayed on the monitor 261, the sending part 214 of the client apparatus 200 transmits an identifier for identifying input information and selected items to the cancer type-evaluating apparatus 100, thereby transmitting the amino acid concentration data of the individual as the subject to the cancer type-evaluating apparatus 100 (step SA-21). In the step SA-21, the transmission of the amino acid concentration data may be realized for example by using an existing file transfer technology such as FTP (File Transfer Protocol).

Then, the request-interpreting part 102a of the cancer type-evaluating apparatus 100 interprets the identifier transmitted from the client apparatus 200 thereby interpreting the requests from the client apparatus 200, and requests the database apparatus 400 to send the multivariate discriminant group for the evaluation of the cancer type (specifically, for example, for the multiple-group discrimination of which of the previously established types of cancers the individual has).

Then, the request-interpreting part 402a in the database apparatus 400 interprets the transmission requests from the cancer type-evaluating apparatus 100 and transmits, to the cancer type-evaluating apparatus 100, the multivariate discriminant group composed of one or a plurality of the multivariate discriminants containing at least one of Glu, ABA, Val, Met, Pro, Phe, Thr, Ile, Leu, and His as the explanatory variables (for example, the updated newest multivariate discriminants) stored in a predetermined region of the memory device 406 (step SA-22).

In step SA-22, each of the multivariate discriminants composing the multivariate discriminant group transmitted to the cancer type-evaluating apparatus 100 may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

The cancer type-evaluating apparatus 100 receives, in the receiving part 102f, the amino acid concentration data of the individual transmitted from the client apparatuses 200 and the multivariate discriminant group transmitted from the database apparatus 400, and stores the received amino acid concentration data in a predetermined memory region of the amino acid concentration data file 106b and each multivariate discriminant composing the received multivariate discriminant group in a predetermined memory region of the multivariate discriminant file 106e4 (step SA-23).

Then, the control device 102 in the cancer type-evaluating apparatus 100 removes data such as defective and outliers from the amino acid concentration data of the individual received in step SA-23 (step SA-24).

Then, the cancer type-evaluating apparatus 100 calculates the discriminant value that is the value of the multivariate discriminant, in the discriminant value-calculating part 102i for each of the multivariate discriminants composing the multivariate discriminant group, based on both (a) the multivariate discriminant group received in step SA-23 and (b) the concentration value of at least one of Glu, ABA, Val, Met, Pro, Phe, Thr, Ile, Leu, and His contained in the amino acid concentration data of the individual from which the data such as the defective and outliers have been removed in step SA-24 (step SA-25).

Then, the discriminant value criterion-discriminating part 102j1 in the cancer type-evaluating apparatus 100 compares the discriminant value group composed of one or a plurality of the discriminant values calculated in step SA-25 with a previously established threshold (cutoff value), thereby discriminating the cancer in the individual out of the previously established types of cancers (specifically, at least two cancers of colon cancer, breast cancer, prostatic cancer, thyroid cancer, lung cancer, gastric cancer, and uterine cancer (more specifically, at least three cancers of colon cancer, breast cancer, prostatic cancer, thyroid cancer, and lung cancer)), and the discrimination results are stored in a predetermined memory region of the evaluation result file 106g (step SA-26).

Then, the sending part 102m in the cancer type-evaluating apparatus 100 sends, to the client apparatus 200 that has sent the amino acid concentration data and to the database apparatus 400, the discrimination results (the discrimination results on which of the cancers the individual has) obtained in step SA-26 (step SA-27). Specifically, the cancer type-evaluating apparatus 100 first generates a Web page for displaying the discrimination results in the Web page-generating part 102e and stores the Web data corresponding to the generated Web page in a predetermined memory region of the memory device 106. Then, the user is authenticated as described above by inputting a predetermined URL (Uniform Resource Locator) into the Web browser 211 of the client apparatus 200 via the input device 250, and the client apparatus 200 sends a Web page browsing request to the cancer type-evaluating apparatus 100. The cancer type-evaluating apparatus 100 then interprets the browsing request transmitted from the client apparatus 200 in the browsing processing part 102b and reads the Web data corresponding to the Web page for displaying the discrimination results, out of the predetermined memory region of the memory device 106. The sending part 102m in the cancer type-evaluating apparatus 100 then sends the read-out Web data to the client apparatus 200 and simultaneously sends the Web data or the discrimination results to the database apparatus 400.

In step SA-27, the control device 102 in the cancer type-evaluating apparatus 100 may notify the discrimination results to the user client apparatus 200 by electronic mail. Specifically, the electronic mail-generating part 102d in the cancer type-evaluating apparatus 100 first acquires the user electronic mail address by referencing the user information stored in the user information file 106a based on the user ID and the like at the transmission timing. The electronic mail-generating part 102d in the cancer type-evaluating apparatus 100 then generates electronic mail data with the acquired electronic mail address as its mail address, including the user name and the discrimination results. The sending part 102m in the cancer type-evaluating apparatus 100 then sends the generated electronic mail data to the user client apparatus 200.

Also in step SA-27, the cancer type-evaluating apparatus 100 may send the discrimination results to the user client apparatus 200 by using, for example, an existing file transfer technology such as FTP.

Returning to FIG. 21, the control device 402 in the database apparatus 400 receives the discrimination results or the Web data transmitted from the cancer type-evaluating apparatus 100 and stores (accumulates) the received discrimination results or the received Web data in a predetermined memory region of the memory device 406 (step SA-28).

The receiving part 213 of the client apparatus 200 receives the Web data transmitted from the cancer type-evaluating apparatus 100, and the received Web data is interpreted with the Web browser 211, to display on the monitor 261 the Web page screen displaying the discrimination result of the individual (step SA-29). When the discrimination results are sent from the cancer type-evaluating apparatus 100 by electronic mail, the electronic mail transmitted from the cancer type-evaluating apparatus 100 is received at any timing, and the received electronic mail is displayed on the monitor 261 with the known function of the electronic mailer 212 in the client apparatus 200.

In this way, the user can confirm the discrimination results of the individual on the multiple-group discrimination of cancer, by browsing the Web page displayed on the monitor 261. The user may print out the content of the Web page displayed on the monitor 261 by the printer 262.

When the discrimination results are transmitted by electronic mail from the cancer type-evaluating apparatus 100, the user reads the electronic mail displayed on the monitor 261, whereby the user can confirm the discrimination results of the individual on the multiple-group discrimination of cancer. The user may print out the content of the electronic mail displayed on the monitor 261 by the printer 262.

Given the foregoing description, the explanation of the cancer evaluation service processing is finished.

### 2-4. Summary of the Second Embodiment and Other Embodiments

According to the cancer-evaluating system described above in detail, the client apparatus 200 sends the amino acid concentration data of the individual to the cancer type-evaluating apparatus 100. Upon receiving the requests from the cancer type-evaluating apparatus 100, the database apparatus 400 transmits the multivariate discriminant group (the multivariate discriminant group composed of one or a plurality of the multivariate discriminants containing at least one of Glu, ABA, Val, Met, Pro, Phe, Thr, Ile, Leu, and His as the explanatory variable) for the multiple-group discrimination of cancer to the cancer type-evaluating apparatus 100. By the cancer type-evaluating apparatus 100, (1) the amino acid concentration data is received from the client apparatus 200, and the multivariate discriminant group is received from the database apparatus 400 simultaneously, (2) the discriminant value that is the value of the multivariate discriminant is calculated for each of the multivariate discriminants composing the multivariate discriminant group, based on both (a) the concentration value of at least one of Glu, ABA, Val, Met, Pro, Phe, Thr, Ile, Leu, and His contained in the received amino acid concentration data and (b) the received multivariate discriminant group, (3) the discriminant value group composed of one or a plurality of the calculated discriminant value is compared with the previously established threshold, thereby discriminating the cancer in the individual out of the previously established types of cancers, and (4) the discrimination results are transmitted to the client apparatus 200 and database apparatus 400. Then, the client apparatus 200 receives and displays the discrimination results transmitted from the cancer type-evaluating apparatus 100, and the database apparatus 400 receives and stores the discrimination results transmitted from the cancer type-evaluating apparatus 100. Thus, a discriminant value group obtained in a multivariate discriminant group useful for a multiple-group discrimination of cancer can be utilized to bring about an effect of enabling accurately the multiple-group discrimination of cancer.

According to the cancer-evaluating system, each of the multivariate discriminants composing the multivariate discriminant group may be any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree. Specifically, the multivariate discriminant group may be any one of following discriminant groups 1 to 16. Thus, a discriminant value group obtained in a multivariate discriminant group useful particularly for a multiple-group discrimination of cancer can be utilized to bring about an effect of enabling more accurately the multiple-group discrimination of cancer.

discriminant group 1: five linear expressions with age, sex, Thr, Glu, Gln, Pro, Cit, ABA, Val, Met, Ile, Leu, Tyr, Phe, His, Orn, Lys, and Arg as the explanatory variables

discriminant group 2: four linear expressions with age, Glu, Pro, Cit, ABA, Met, Ile, Leu, Phe, His, Trp, Orn, and Lys as the explanatory variables

discriminant group 3: four linear expressions with age, Thr, Glu, Gln, Pro, ABA, Val, Met, Ile, Leu, Phe, His, and Arg as the explanatory variables

discriminant group 4: four linear expressions with age, sex, Thr, Glu, Pro, ABA, Val, Met, Ile, Leu, Phe, and His as the explanatory variables

discriminant group 5: three linear expressions with age, Asn, Glu, ABA, Val, Phe, His, and Trp as the explanatory variables

discriminant group 6: three linear expressions with age, Thr, Glu, Pro, Val, Met, Ile, Leu, His, and Arg as the explanatory variables

discriminant group 7: four linear expressions with age, sex, Thr, Glu, Gln, Pro, Cit, ABA, Val, Met, Ile, Leu, Tyr, Phe, Orn, and Arg as the explanatory variables

discriminant group 8: three linear expressions with age, Asn, Glu, ABA, Val, Phe, His, and Trp as the explanatory variables

discriminant group 9: three linear expressions with age, Thr, Glu, Gln, Pro, ABA, Val, Met, Ile, Phe, and Arg as the explanatory variables

discriminant group 10: three linear expressions with age, sex, Thr, Glu, Pro, ABA, Val, and Met as the explanatory variables

discriminant group 11: two linear expressions with age, Cit, ABA, Val, and Met as the explanatory variables

discriminant group 12: two linear expressions with age, Thr, Glu, Pro, Met, and Phe as the explanatory variables

discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables

discriminant group 14: two linear expressions with Glu, Gln, ABA, Val, Ile, Phe, and Arg as the explanatory variables

discriminant group 15: two linear expressions with Thr, Glu, Gln, ABA, Ile, Leu, and Arg as the explanatory variables

discriminant group 16: two fractional expressions with Thr, Gln, Ala, Cit, ABA, Ile, His, Orn, and Arg as the explanatory variables

Each multivariate discriminant composing these multivariate discriminant groups can be prepared by a method described in International Publication WO 2004/052191 that is an international application filed by the present applicant or by a method (multivariate discriminant-preparing processing described later) described in International Publication WO 2006/098192 that is an international application filed by the present applicant. Any multivariate discriminants obtained by these methods can be preferably used in the evaluation of the cancer type, regardless of the unit of the amino acid concentration in the amino acid concentration data as input data.

In addition to the second embodiment described above, the cancer type-evaluating apparatus, the cancer-evaluating method, the cancer-evaluating system, the cancer-evaluating program product and the recording medium according to the present invention can be practiced in various different embodiments within the technological scope of the claims. For example, among the processings described in the second embodiment above, all or a part of the processings described above as performed automatically may be performed manually, and all or a part of the manually conducted processings may be performed automatically by known methods. In addition, the processing procedure, control procedure, specific name, various registered data, information including parameters such as retrieval condition, screen, and database configuration shown in the description above or drawings may be modified arbitrarily, unless specified otherwise. For example, the components of the cancer type-evaluating apparatus 100 shown in the figures are conceptual and functional and may not be the same physically as those shown in the figure. In addition, all or an arbitrary part of the operational function of each component and each device in the cancer type-evaluating apparatus 100 (in particular, the operational functions executed in the control device 102) may be executed by the CPU (Central Processing Unit) or the programs executed by the CPU, and may be realized as wired-logic hardware.

The “program” is a data processing method written in any language or by any description method and may be of any format such as source code or binary code. The “program” may not be limited to a program configured singly, and may include a program configured decentrally as a plurality of modules or libraries, and a program to achieve the function together with a different program such as OS (Operating System). The program is stored on a recording medium and read mechanically as needed by the cancer type-evaluating apparatus 100. Any well-known configuration or procedure may be used as specific configuration, reading procedure, installation procedure after reading, and the like for reading the programs recorded on the recording medium in each apparatus.

The “recording media” includes any “portable physical media”, “fixed physical media”, and “communication media”. Examples of the “portable physical media” include flexible disk, magnetic optical disk, ROM, EPROM (Erasable Programmable Read Only Memory), EEPROM (Electronically Erasable and Programmable Read Only Memory), CD-ROM (Compact Disk Read Only Memory), MO (Magneto-Optical disk), DVD (Digital Versatile Disk), and the like. Examples of the “fixed physical media” include ROM, RAM, HD, and the like which are installed in various computer systems. The “communication media” for example stores the program for a short period of time such as communication line and carrier wave when the program is transmitted via a network such as LAN (Local Area Network), WAN (Wide Area Network), or the Internet.

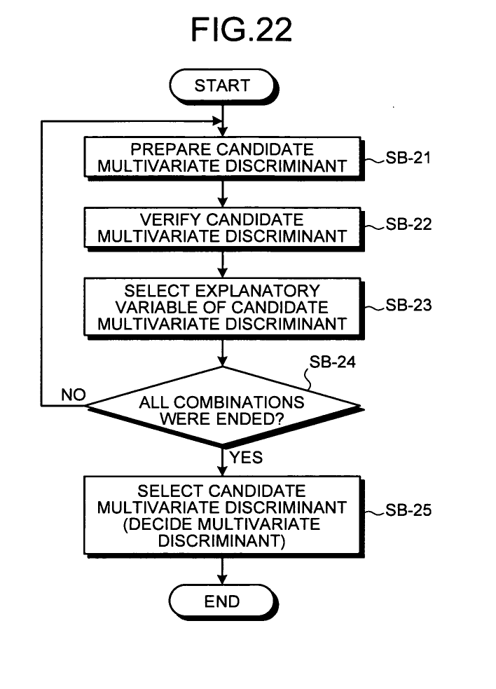

Finally, an example of the multivariate discriminant-preparing processing performed in the cancer type-evaluating apparatus 100 is described in detail with reference to FIG. 22. FIG. 22 is a flowchart showing an example of the multivariate discriminant-preparing processing. The multivariate discriminant-preparing processing is collectively executed to the data obtained by summarizing the cancers (specifically, for example, colon cancer, breast cancer, prostatic cancer, thyroid cancer, lung cancer, gastric cancer, and uterine cancer described above) being a subject when evaluating the cancer type. The multivariate discriminant-preparing processing may be performed in the database apparatus 400 handling the cancer state information.

In the present description, the cancer type-evaluating apparatus 100 stores the cancer state information previously obtained from the database apparatus 400 in a predetermined memory region of the cancer state information file 106c. The cancer type-evaluating apparatus 100 shall store, in a predetermined memory region of the designated cancer state information file 106d, the cancer state information including the cancer state index data and amino acid concentration data designated previously in the cancer state information-designating part 102g.

The candidate multivariate discriminant-preparing part 102h1 in the multivariate discriminant-preparing part 102h first prepares the candidate multivariate discriminant groups according to a predetermined discriminant-preparing method from the cancer state information stored in a predetermine memory region of the designated cancer state information file 106d, and stores the prepared candidate multivariate discriminant groups in a predetermined memory region of the candidate multivariate discriminant file 106e1 (step SB-21). Specifically, the candidate multivariate discriminant-preparing part 102h1 in the multivariate discriminant-preparing part 102h first selects a desired method out of a plurality of different discriminant-preparing methods (including those for multivariate analysis such as principal component analysis, discriminant analysis, support vector machine, multiple regression analysis, logistic regression analysis, k-means method, cluster analysis, and decision tree) and determines the form of the candidate multivariate discriminant group to be prepared based on the selected discriminant-preparing method. The candidate multivariate discriminant-preparing part 102h1 in the multivariate discriminant-preparing part 102h then performs various calculation corresponding to the selected function-selecting method (e.g., average or variance), based on the cancer state information. The candidate multivariate discriminant-preparing part 102h1 in the multivariate discriminant-preparing part 102h then determines the parameters for the calculation result and the determined candidate multivariate discriminant group. In this way, the candidate multivariate discriminant group is generated based on the selected discriminant-preparing method. When the candidate multivariate discriminant groups are generated simultaneously and concurrently (in parallel) by using a plurality of different discriminant-preparing methods in combination, the processings described above may be executed concurrently for each selected discriminant-preparing method. Alternatively when the candidate multivariate discriminant groups are generated in series by using a plurality of different discriminant-preparing methods in combination, for example, the candidate multivariate discriminant groups may be generated by converting the cancer state information with the candidate multivariate discriminant groups prepared by performing principal component analysis and performing discriminant analysis of the converted cancer state information.

The candidate multivariate discriminant-verifying part 102h2 in the multivariate discriminant-preparing part 102h verifies (mutually verifies) the candidate multivariate discriminant group prepared in the step SB-21 according to a particular verifying method and stores the verification result in a predetermined memory region of the verification result file 106e2 (step SB-22). Specifically, the candidate multivariate discriminant-verifying part 102h2 in the multivariate discriminant-preparing part 102h first generates the verification data to be used in verification of the candidate multivariate discriminant group, based on the cancer state information stored in a predetermined memory region of the designated cancer state information file 106d, and verifies the candidate multivariate discriminant group according to the generated verification data. If a plurality of the candidate multivariate discriminant groups is generated by using a plurality of different discriminant-preparing methods in the step SB-21, the candidate multivariate discriminant-verifying part 102h2 in the multivariate discriminant-preparing part 102h verifies each candidate multivariate discriminant group corresponding to each discriminant-preparing method according to a particular verifying method. Here in the step SB-22, at least one of the discrimination rate, sensitivity, specificity, information criterion, and the like of the candidate multivariate discriminant group may be verified based on at least one method of the bootstrap method, holdout method, leave-one-out method, and the like. Thus, it is possible to select the candidate multivariate discriminant group higher in predictability or reliability, by taking the cancer state information and diagnostic condition into consideration.

Then, the explanatory variable-selecting part 102h3 in the multivariate discriminant-preparing part 102h selects the combination of the amino acid concentration data contained in the cancer state information used in preparing the candidate multivariate discriminant group by selecting the explanatory variable of the candidate multivariate discriminant group from the verification result obtained in the step SB-22 according to a predetermined explanatory variable-selecting method, and stores the cancer state information including the selected combination of the amino acid concentration data in a predetermined memory region of the selected cancer state information file 106e3 (step SB-23). When a plurality of the candidate multivariate discriminant groups is generated by using a plurality of different discriminant-preparing methods in the step SB-21 and each candidate multivariate discriminant group corresponding to each discriminant-preparing method is verified according to a predetermined verifying method in the step SB-22, the explanatory variable-selecting part 102h3 in the multivariate discriminant-preparing part 102h selects the explanatory variable of the candidate multivariate discriminant group for each candidate multivariate discriminant group corresponding to the verification result obtained in the step SB-22, according to a predetermined explanatory variable-selecting method in the step SB-23. Here in the step SB-23, the explanatory variable of the candidate multivariate discriminant group may be selected from the verification results according to at least one of the stepwise method, best path method, local search method, and genetic algorithm. The best path method is a method of selecting an explanatory variable by optimizing an evaluation index of the candidate multivariate discriminant group while eliminating the explanatory variables contained in the candidate multivariate discriminant group one by one. In the step SB-23, the explanatory variable-selecting part 102h3 in the multivariate discriminant-preparing part 102h may select the combination of the amino acid concentration data based on the cancer state information stored in a predetermined memory region of the designated cancer state information file 106d.

The multivariate discriminant-preparing part 102h then judges whether all combinations of the amino acid concentration data contained in the cancer state information stored in a predetermined memory region of the designated cancer state information file 106d are processed, and if the judgment result is “End” (Yes in step SB-24), the processing advances to the next step (step SB-25), and if the judgment result is not “End” (No in step SB-24), it returns to the step SB-21. The multivariate discriminant-preparing part 102h may judge whether the processing is performed a predetermined number of times, and if the judgment result is “End” (Yes in step SB-24), the processing may advance to the next step (step SB-25), and if the judgment result is not “End” (No in step SB-24), it may return to the step SB-21. The multivariate discriminant-preparing part 102h may judge whether the combination of the amino acid concentration data selected in the step SB-23 is the same as the combination of the amino acid concentration data contained in the cancer state information stored in a predetermined memory region of the designated cancer state information file 106d or the combination of the amino acid concentration data selected in the previous step SB-23, and if the judgment result is “the same” (Yes in step SB-24), the processing may advance to the next step (step SB-25) and if the judgment result is not “the same” (No in step SB-24), it may return to the step SB-21. If the verification result is specifically the evaluation value for each multivariate discriminant group, the multivariate discriminant-preparing part 102h may advance to the step SB-25 or return to the step SB-21, based on the comparison of the evaluation value with a particular threshold corresponding to each discriminant-preparing method.

Then, the multivariate discriminant-preparing part 102h determines the multivariate discriminant group by selecting the candidate multivariate discriminant group used as the multivariate discriminant group based on the verification results from a plurality of the candidate multivariate discriminant groups, and stores the determined multivariate discriminant group (the selected candidate multivariate discriminant group) in particular memory region of the multivariate discriminant file 106e4 (step SB-25). Here, in the step SB-25, for example, there are cases where the optimal multivariate discriminant group is selected from the candidate multivariate discriminant groups prepared in the same discriminant-preparing method or the optimal multivariate discriminant group is selected from all candidate multivariate discriminant groups.

Given the foregoing description, the explanation of the multivariate discriminant-preparing processing is finished.

### Example 1

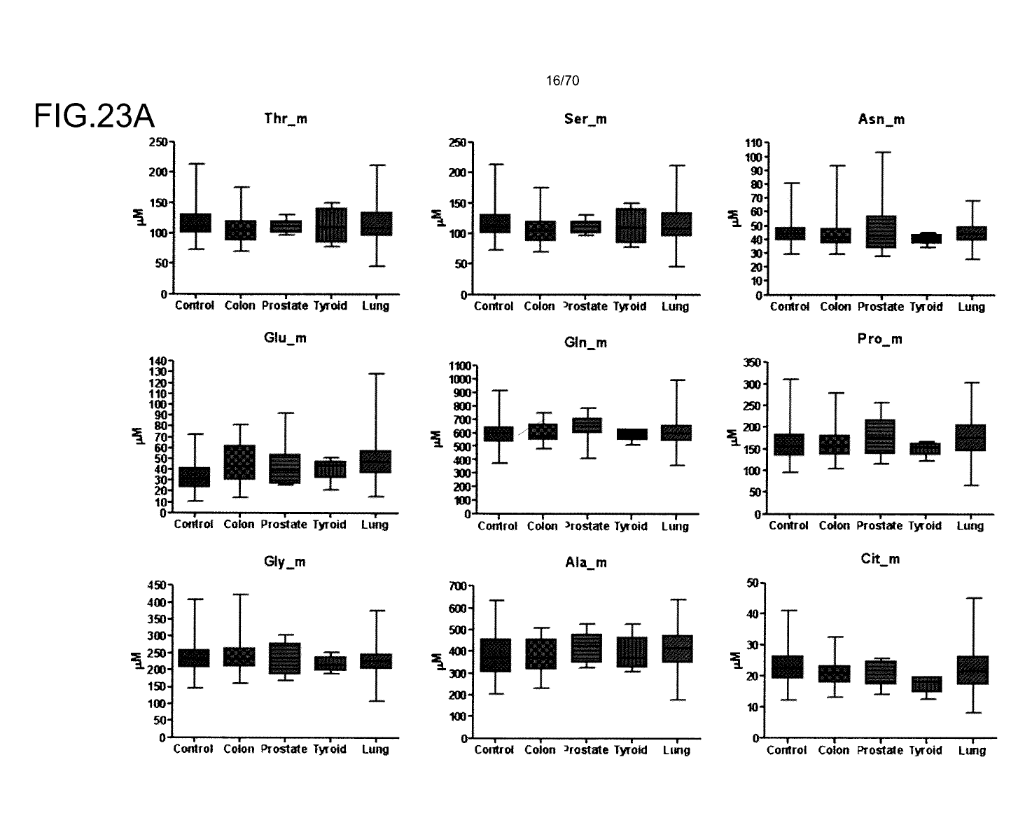

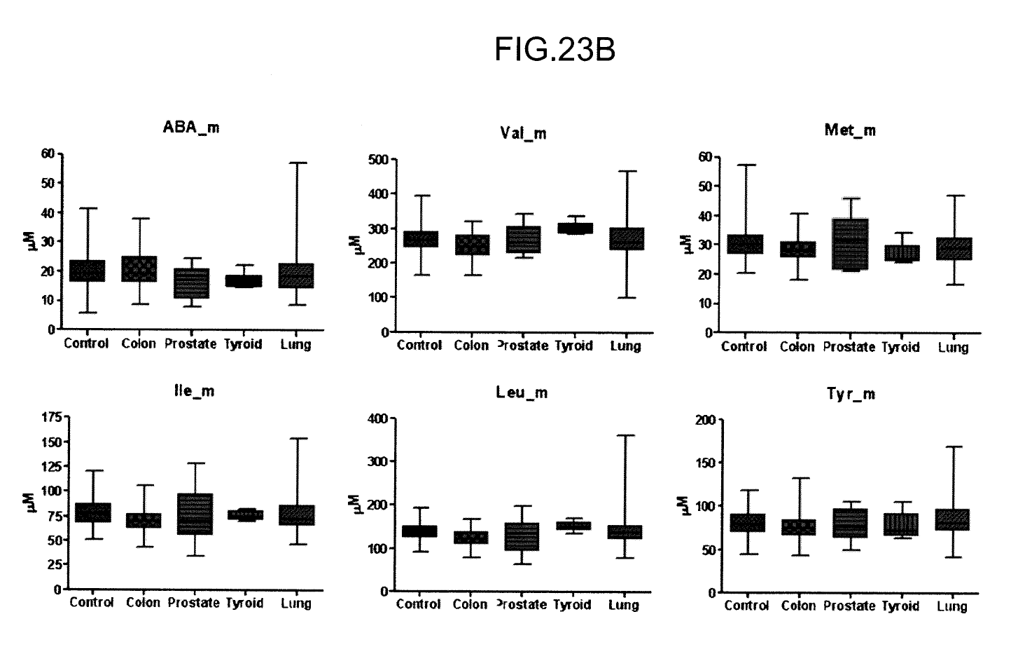

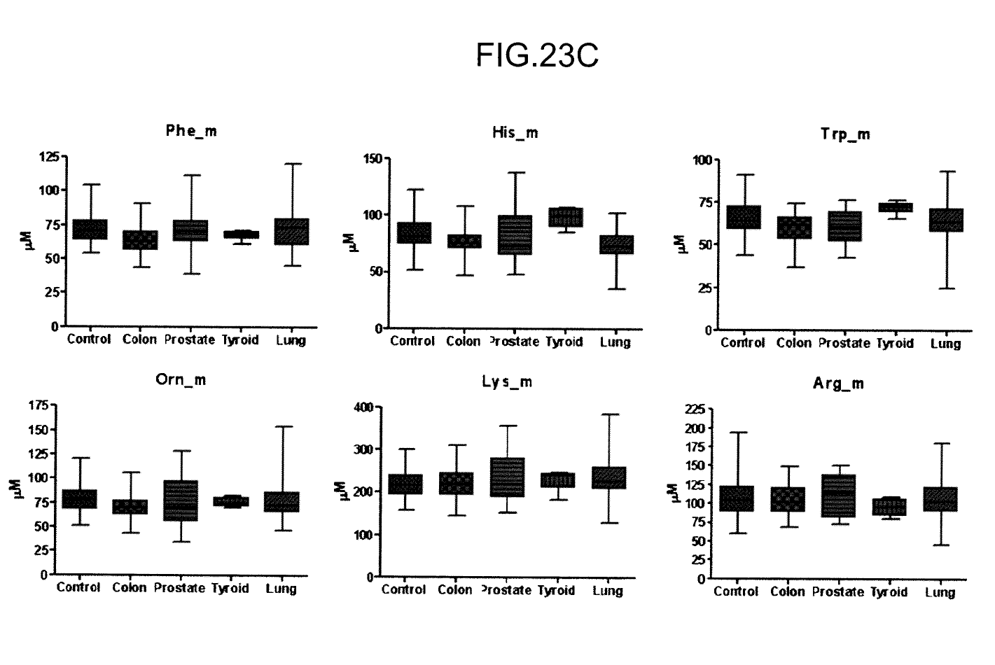

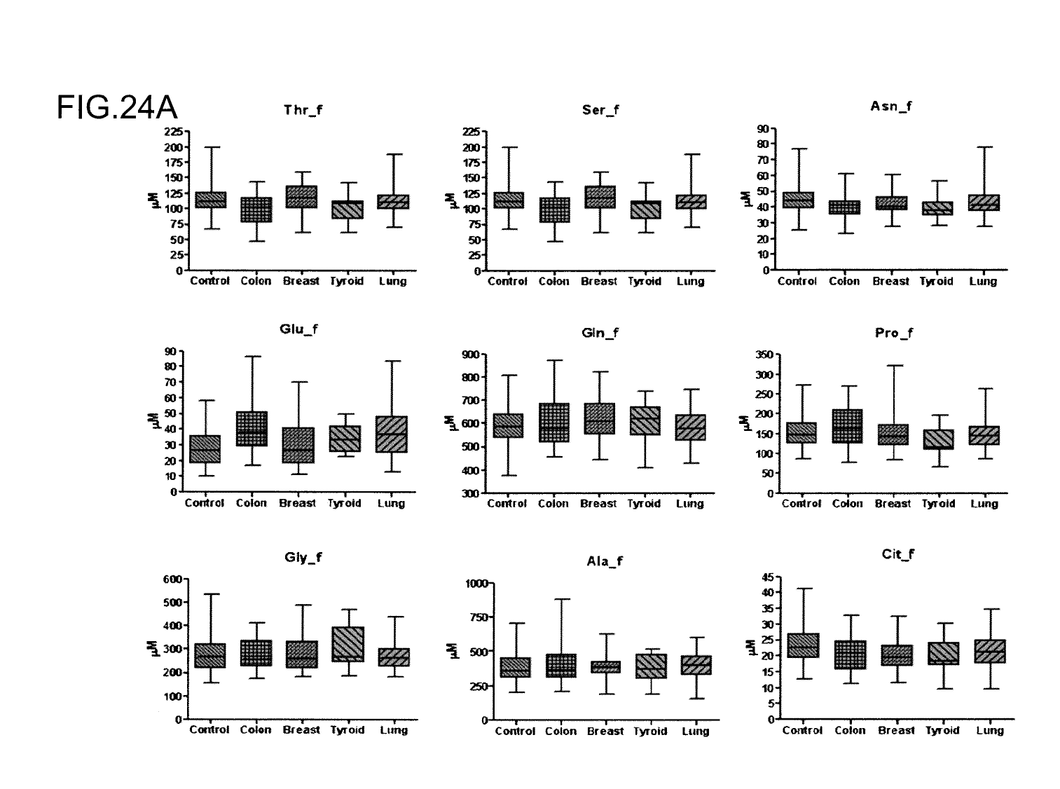

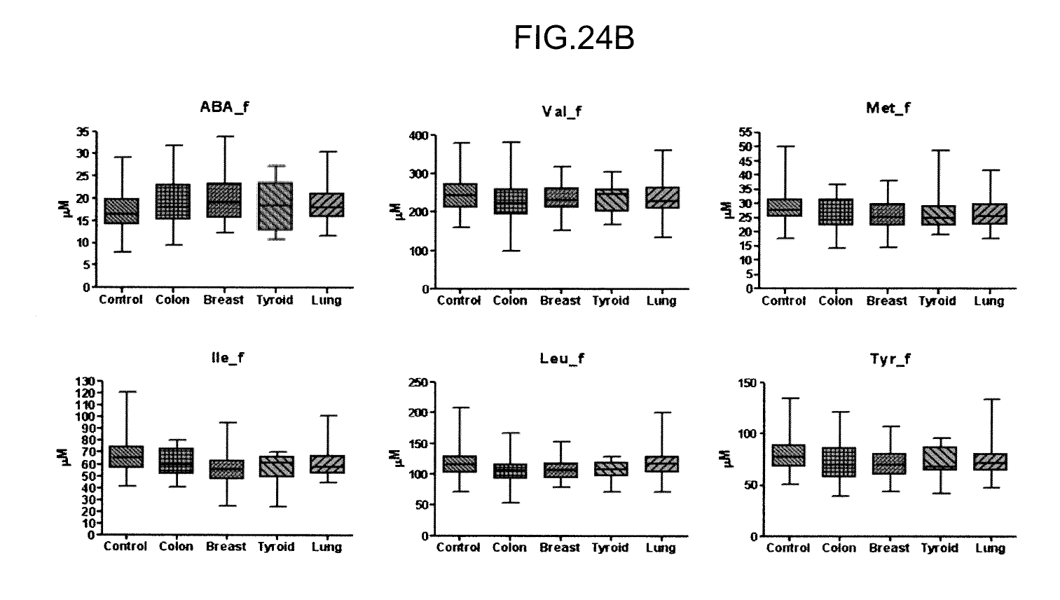

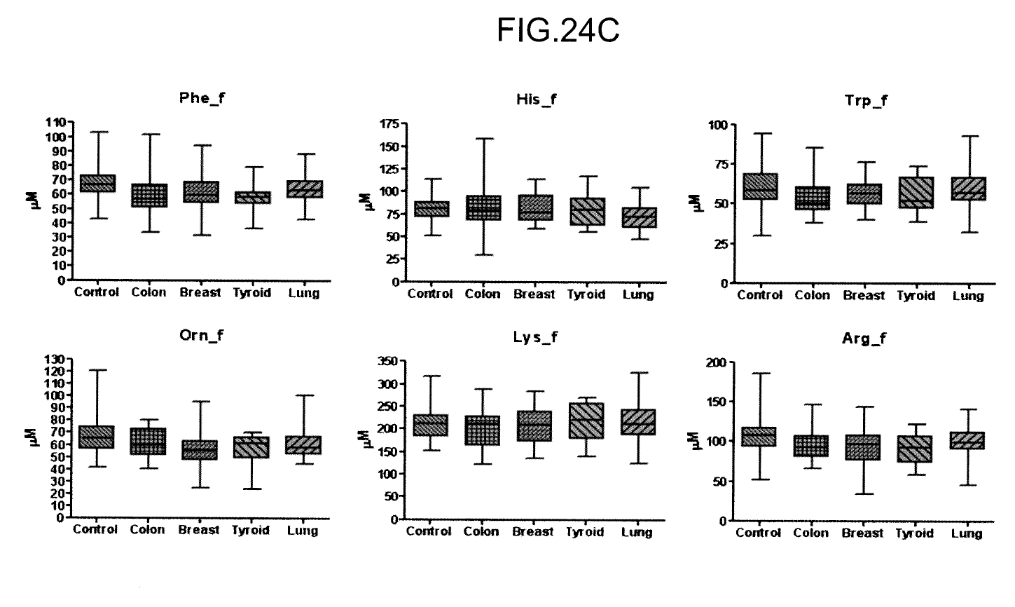

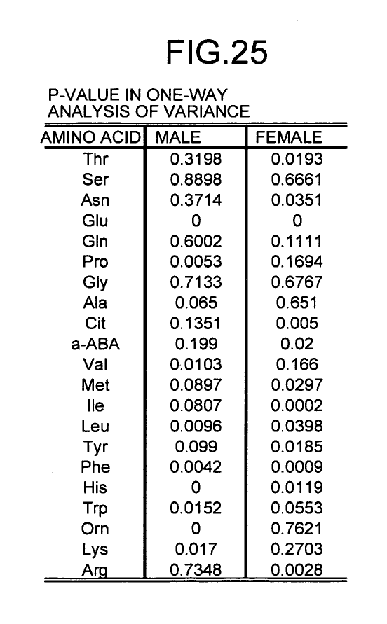

Amino acid concentration in blood is measured by the amino acid analysis method in blood samples of various cancer patient groups with definitive diagnosis of cancer and blood samples of a cancer-free group. The unit of the amino acid concentration is nmol/ml. FIGS. 23 and 24 are boxplots showing the distribution of amino acid explanatory variables of various cancer patients and cancer-free subjects. FIG. 23 is the boxplots showing the distribution of the amino acid explanatory variables of male various cancer patients and male cancer-free subjects, and FIG. 24 is the boxplots showing the distribution of the amino acid explanatory variables of female various cancer patients and female cancer-free subjects. In FIGS. 23 and 24, the horizontal axis indicates the cancer-free group and the various cancer groups, and ABA in the figures represents α-ABA (α-aminobutyric acid). Further, for the purpose of discrimination among the various cancer groups and the cancer-free group, evaluation using one-way analysis of variance is carried out with respect to the discrimination among the various cancer groups and the cancer-free group by each amino acid explanatory variable, and p-values of the amino acid explanatory variables Glu, Pro, Val, Leu, Phe, His, Trp, Orn, and Lys are smaller than 0.05 in male data, and p-values of the amino acid explanatory variables Asn, Glu, Pro, Cit, ABA, Met, Ile, Leu, Tyr, Phe, His, and Arg are smaller than 0.05 in female data (FIG. 25). As a result, it is proved that the amino acid explanatory variables Asn, Glu, Pro, Cit, ABA, Val, Met, Ile, Leu, Tyr, Phe, His, Trp, Orn, Lys, and Arg have an ability to discriminate among the multiple groups of the various cancer groups and the cancer-free group.

### Example 2

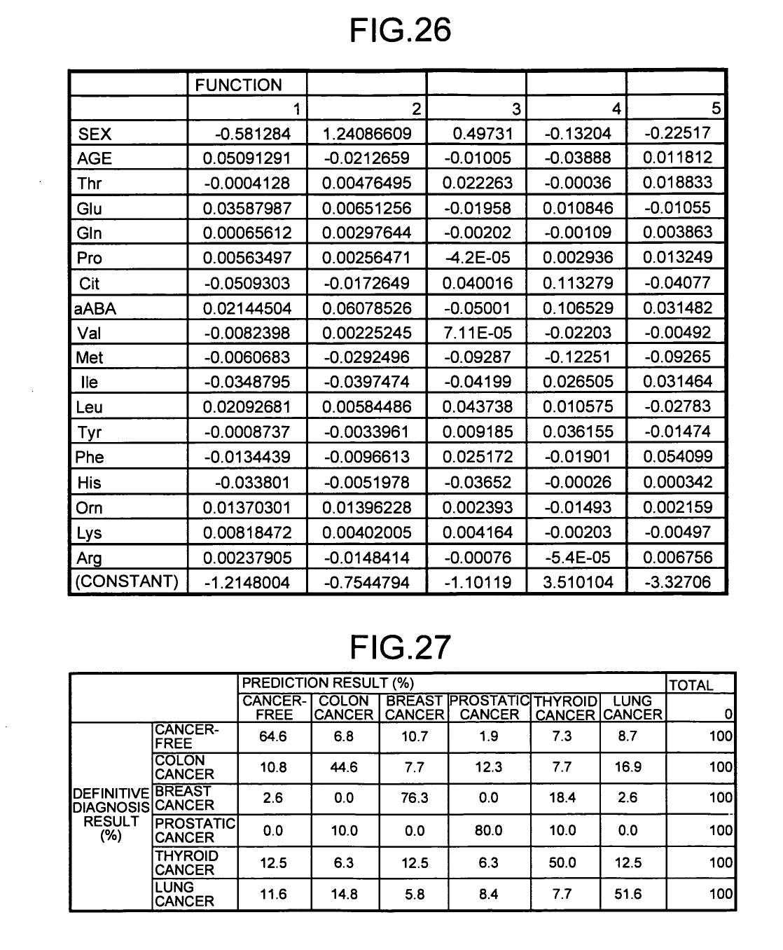

The sample data used in Example 1 is used. Indices to maximize performance to discriminate among six groups of the various cancer groups (colon cancer, breast cancer, prostatic cancer, thyroid cancer, and lung cancer) and the cancer-free group with respect to cancer are searched by linear discriminant analysis using a stepwise explanatory variable selecting method, and a linear discriminant group composed of age, sex (male=1 and female=2), Thr, Glu, Gln, Pro, Cit, ABA, Val, Met, Ile, Leu, Tyr, Phe, His, Orn, Lys, and Arg (the coefficients of the age, the sex, and the amino acid explanatory variables Thr, Glu, Gln, Pro, Cit, ABA, Val, Met, Ile, Leu, Tyr, Phe, His, Orn, Lys, and Arg of each discriminant are presented in FIG. 26) is obtained as an index formula group 1.

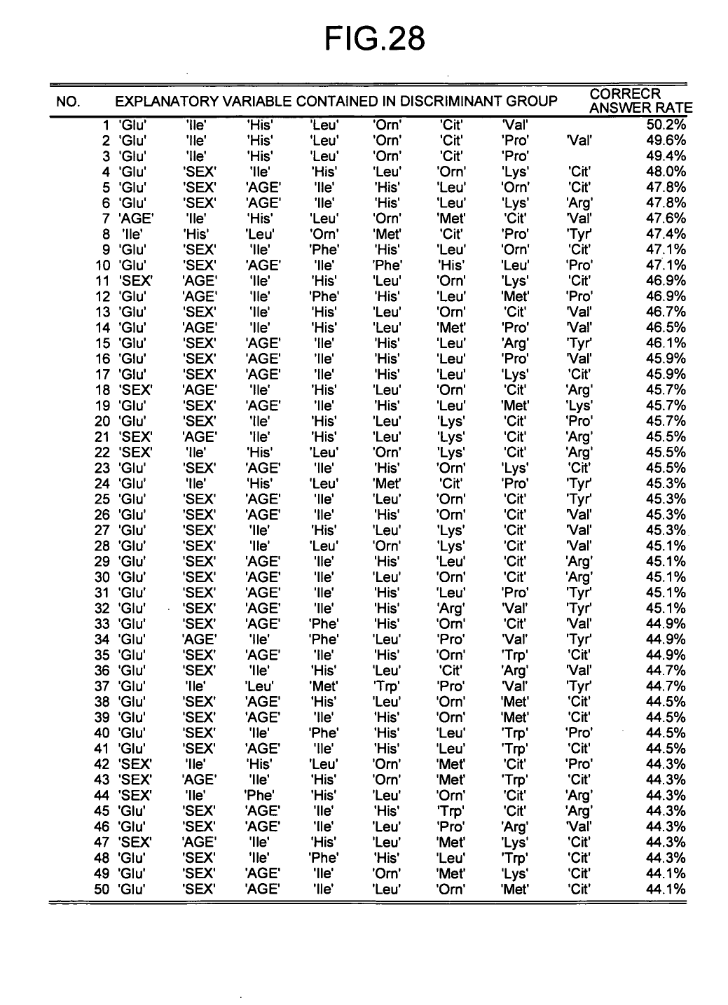

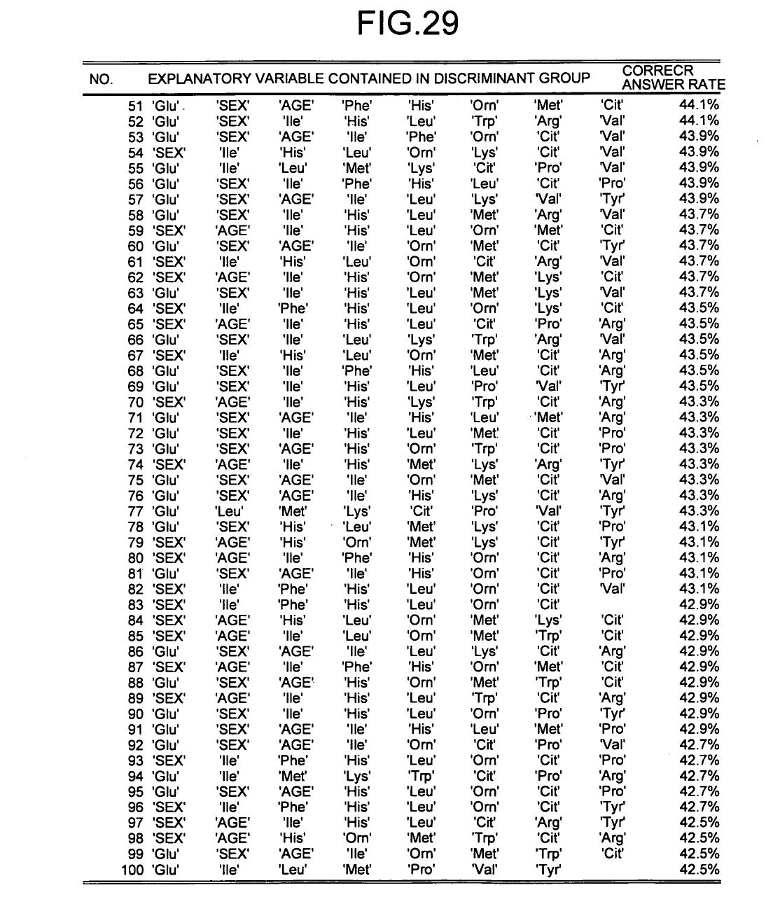

As a result of the evaluation of the diagnosis performance of the various cancers (colon cancer, breast cancer, prostatic cancer, thyroid cancer, and lung cancer) and the cancer-free based on the index formula group 1 by correct answer rates of discrimination results, high discrimination ability is demonstrated such that the correct answer rates of the cancer-free, the colon cancer, the breast cancer, the prostatic cancer, the thyroid cancer, and the lung cancer are 64.6%, 44.6%, 76.3%, 80.0%, 50.0%, and 51.6%, respectively, and the correct answer rate of the total is 58.6% when the prior probability of each is 16.7% (FIG. 27). The value of each coefficient in the discriminant presented in FIG. 26 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 26 is obtained. The list of the explanatory variables contained in the discriminant groups is presented in FIGS. 28 and 29.

### Example 3

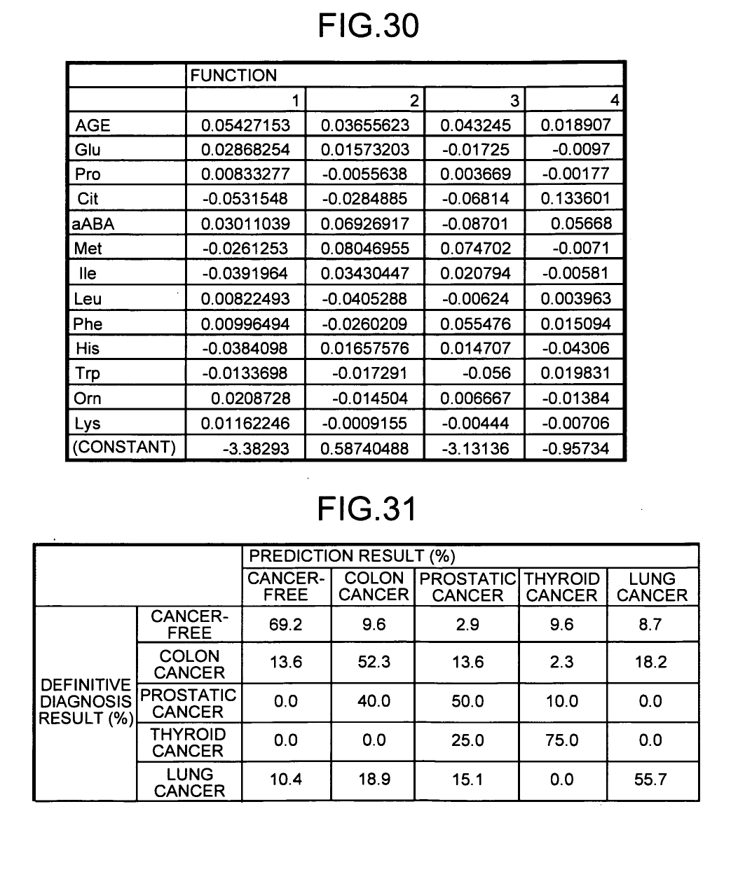

The male data out of the sample data used in Example 1 is used. Indices to maximize performance to discriminate among five groups of the various cancer groups (colon cancer, prostatic cancer, thyroid cancer, and lung cancer) and the cancer-free group with respect to cancer are searched by the linear discriminant analysis using the stepwise explanatory variable selecting method, and a linear discriminant group composed of age, Glu, Pro, Cit, ABA, Met, Ile, Leu, Phe, His, Trp, Orn, and Lys (the coefficients of the age and the amino acid explanatory variables Glu, Pro, Cit, ABA, Met, Ile, Leu, Phe, His, Trp, Orn, and Lys of each discriminant are presented in FIG. 30) is obtained as an index formula group 2.

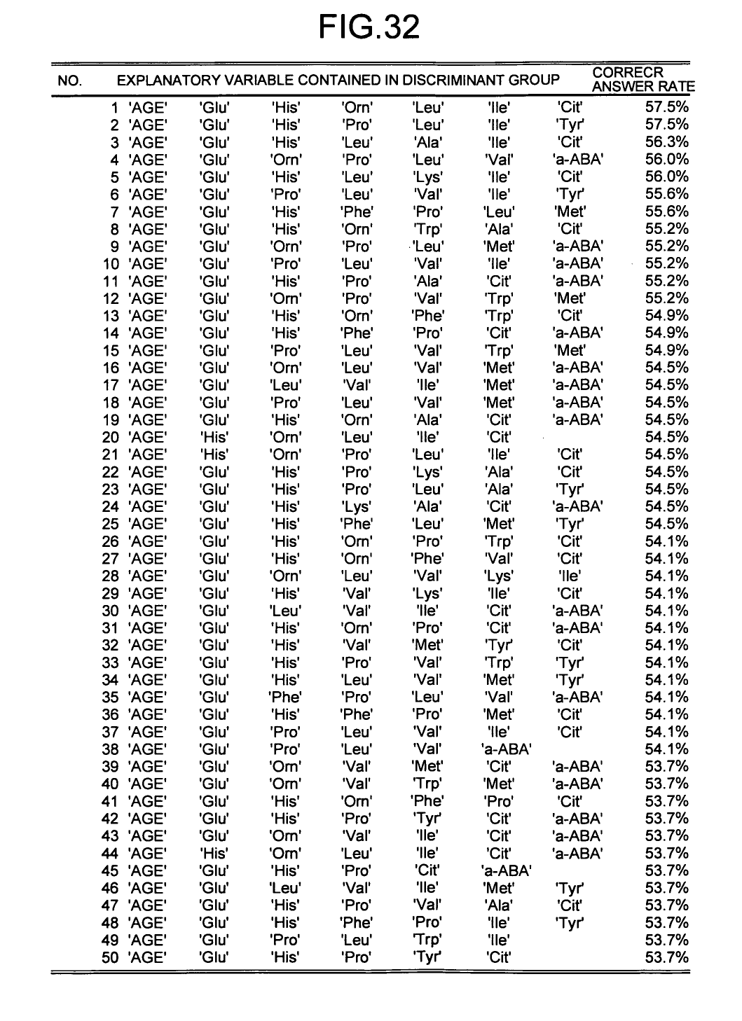

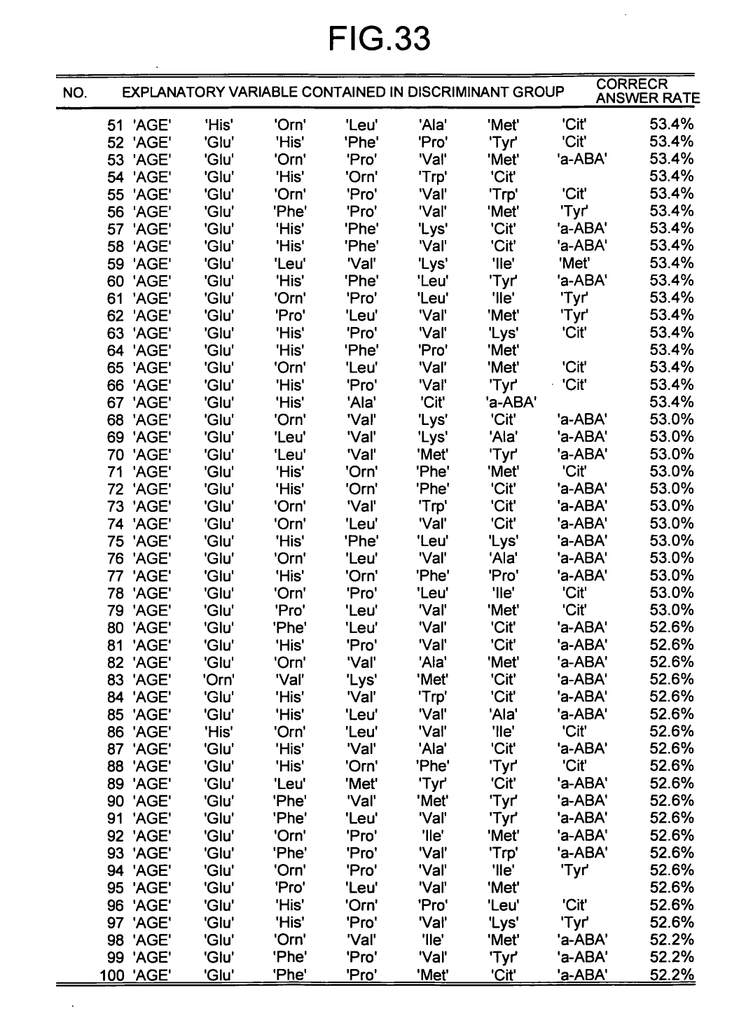

As a result of the evaluation of the diagnosis performance of the various cancers (colon cancer, prostatic cancer, thyroid cancer, and lung cancer) and the cancer-free based on the index formula group 2 by correct answer rates of discrimination results, high discrimination ability is demonstrated such that the correct answer rates of the cancer-free, the colon cancer, the prostatic cancer, the thyroid cancer, and the lung cancer are 69.2%, 52.3%, 50.0%, 75.0%, and 55.7%, respectively, and the correct answer rate of the total is 60.4% when the prior probability of each is 20.0% (FIG. 31). The value of each coefficient in the discriminant presented in FIG. 30 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 30 is obtained. The list of the explanatory variables contained in the discriminant groups is presented in FIGS. 32 and 33.

### Example 4

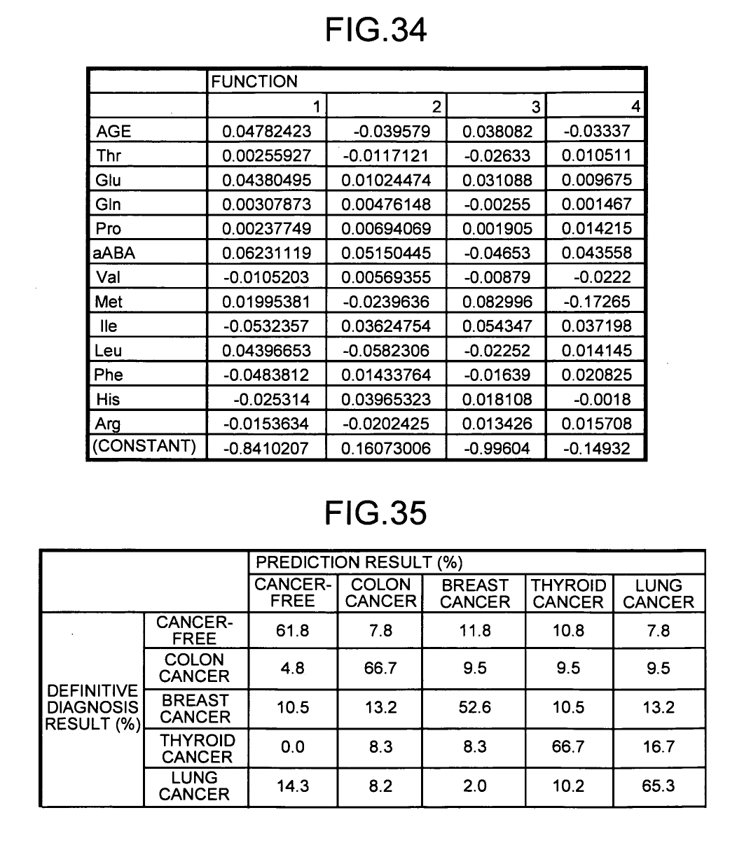

The female data out of the sample data used in Example 1 is used. Indices to maximize performance to discriminate among five groups of the various cancer groups (colon cancer, breast cancer, thyroid cancer, and lung cancer) and the cancer-free group with respect to cancer are searched by the linear discriminant analysis using the stepwise explanatory variable selecting method, and a linear discriminant group composed of age, Thr, Glu, Gln, Pro, ABA, Val, Met, Ile, Leu, Phe, His, and Arg (the coefficients of the age and the amino acid explanatory variables Thr, Glu, Gln, Pro, ABA, Val, Met, Ile, Leu, Phe, His, and Arg of each discriminant are presented in FIG. 34) is obtained as an index formula group 3.

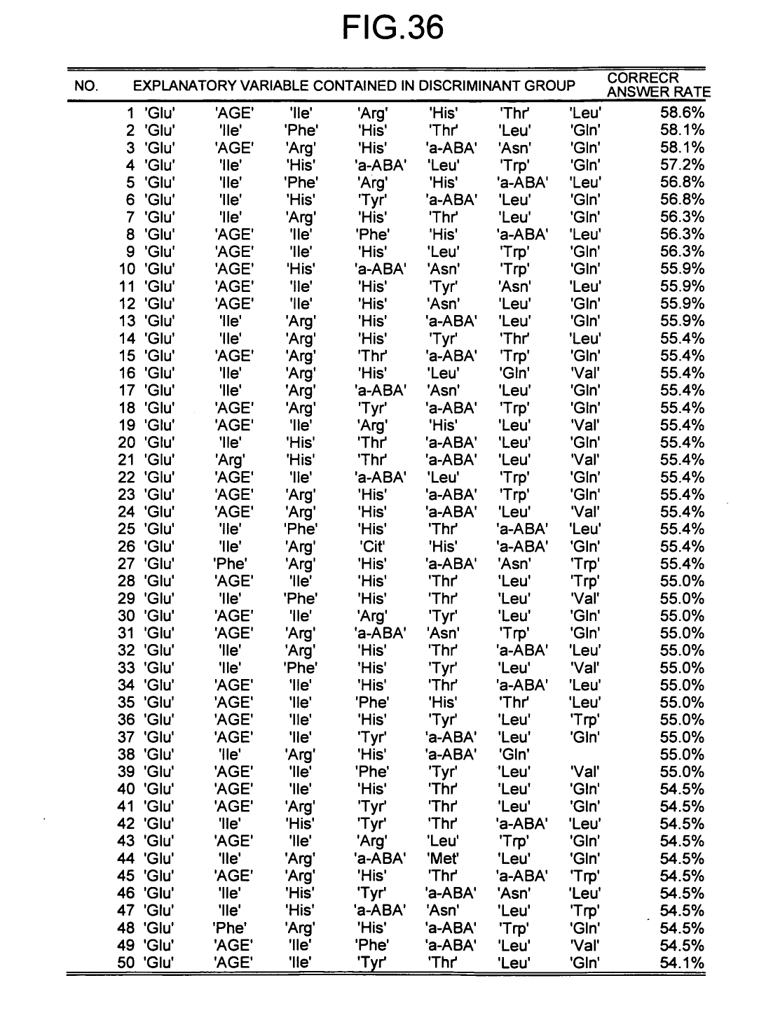

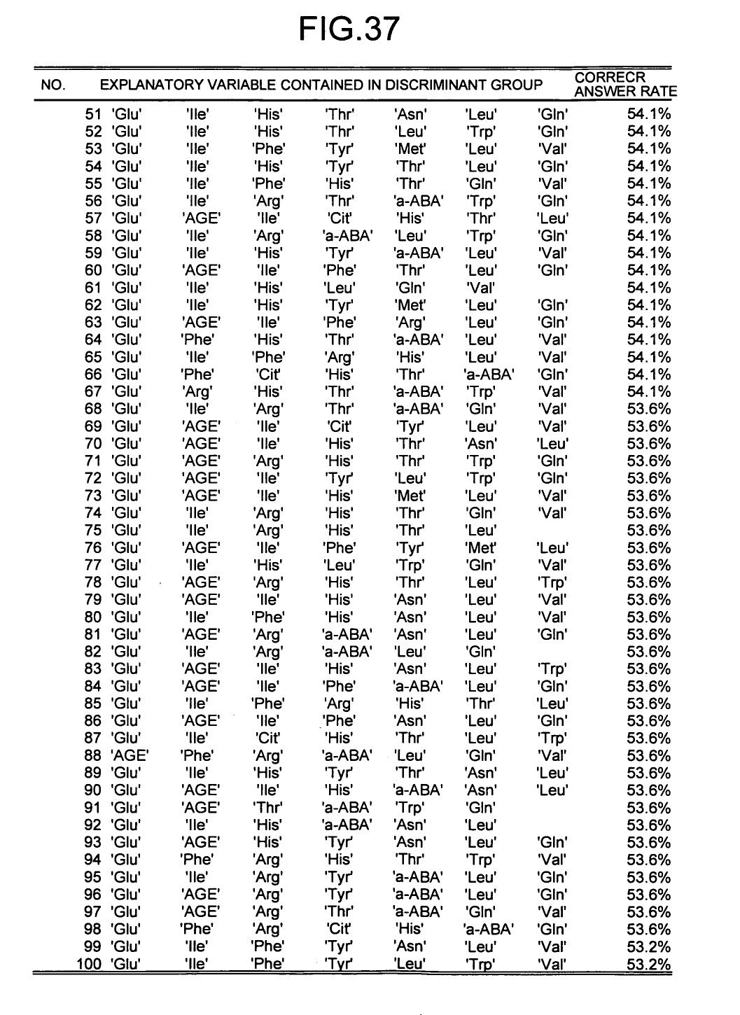

As a result of the evaluation of the diagnosis performance of the various cancers (colon cancer, breast cancer, thyroid cancer, and lung cancer) and the cancer-free based on the index formula group 3 by correct answer rates of discrimination results, high discrimination ability is demonstrated such that the correct answer rates of the cancer-free, the colon cancer, the breast cancer, the thyroid cancer, and the lung cancer are 61.8%, 66.7%, 52.6%, 66.7%, and 65.3%, respectively, and the correct answer rate of the total is 61.7% when the prior probability of each is 20.0% (FIG. 35). The value of each coefficient in the discriminant presented in FIG. 34 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 34 is obtained. The list of the explanatory variables contained in the discriminant groups is presented in FIGS. 36 and 37.

### Example 5

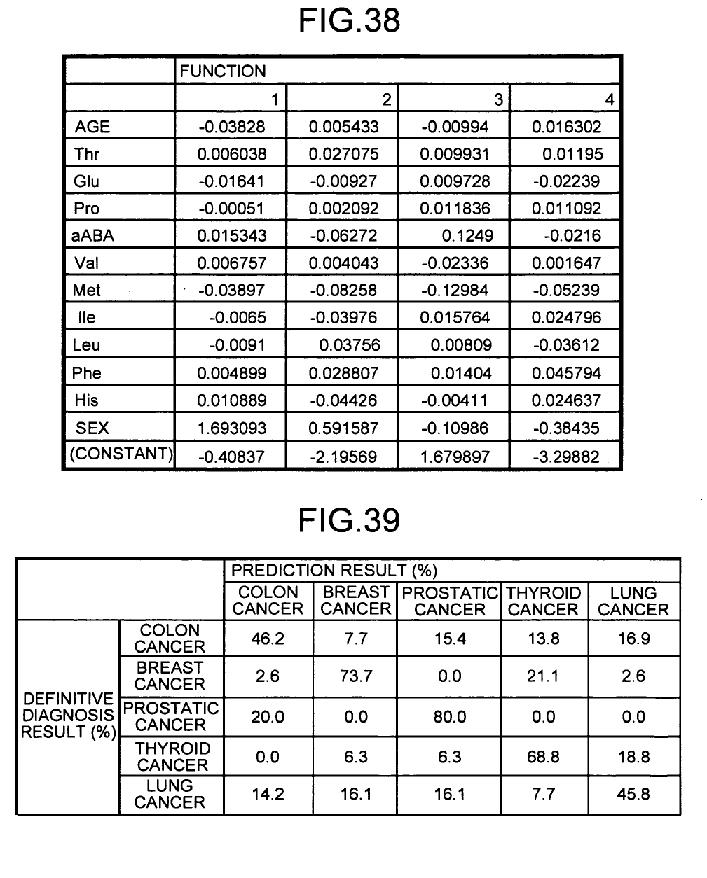

The data of the colon cancer group, the breast cancer group, the prostatic cancer group, the thyroid cancer group and the lung cancer group out of the sample data used in Example 1 is used. Indices to maximize performance to discriminate among five groups of the various cancer groups (colon cancer, breast cancer, prostatic cancer, thyroid cancer, and lung cancer) with respect to cancer are searched by the linear discriminant analysis using the stepwise explanatory variable selecting method, and a linear discriminant group composed of age, sex (male=1 and female=2), Thr, Glu, Pro, ABA, Val, Met, Ile, Leu, Phe, and His (the coefficients of the age, the sex, and the amino acid explanatory variables Thr, Glu, Pro, ABA, Val, Met, Ile, Leu, Phe, and His of each discriminant are presented in FIG. 38) is obtained as an index formula group 4.

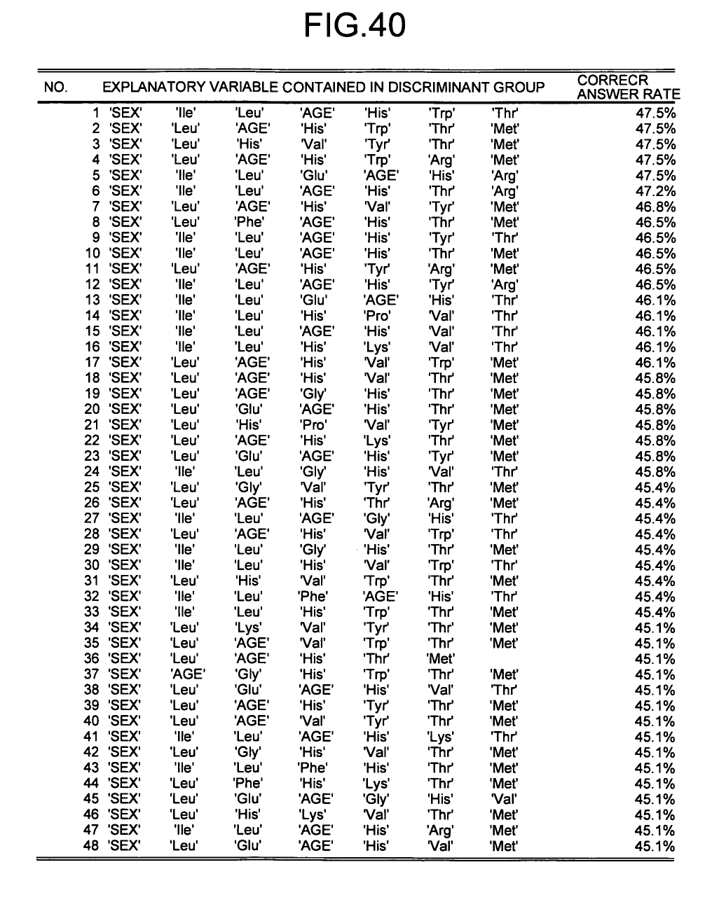

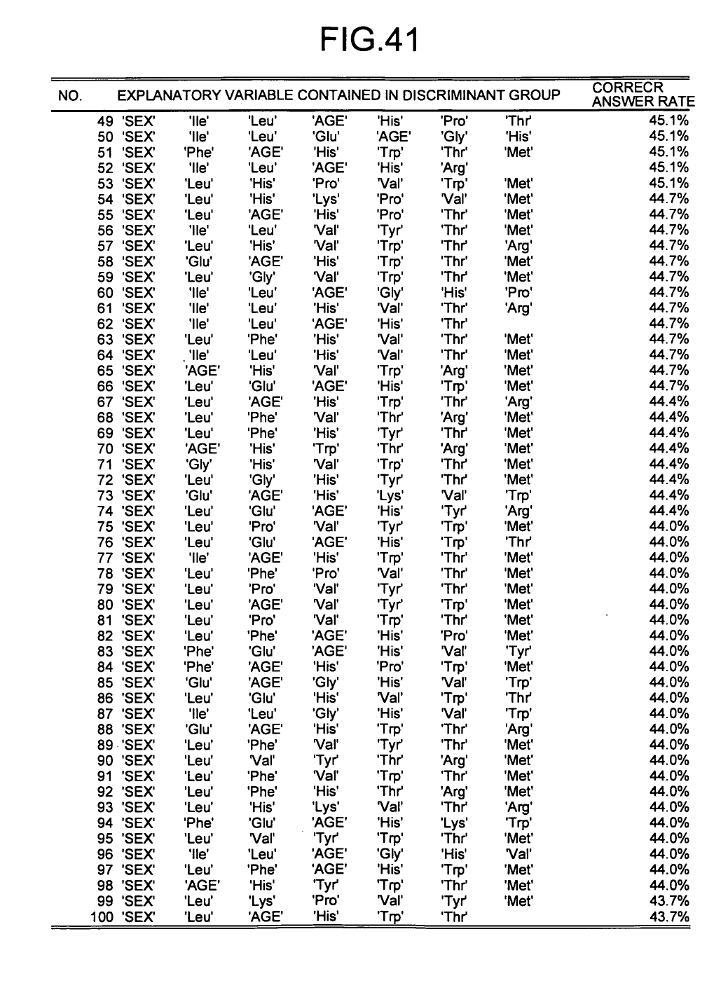

As a result of the evaluation of the diagnosis performance of the various cancers (colon cancer, breast cancer, prostatic cancer, thyroid cancer, and lung cancer) based on the index formula group 4 by correct answer rates of discrimination results, high discrimination ability is demonstrated such that the correct answer rates of the colon cancer, the breast cancer, the prostatic cancer, the thyroid cancer, and the lung cancer are 46.2%, 73.7%, 80.0%, 68.8%, and 45.8%, respectively, and the correct answer rate of the total is 52.1% when the prior probability of each is 20.0% (FIG. 39). The value of each coefficient in the discriminant presented in FIG. 38 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 38 is obtained. The list of the explanatory variables contained in the discriminant groups is presented in FIGS. 40 and 41.

### Example 6

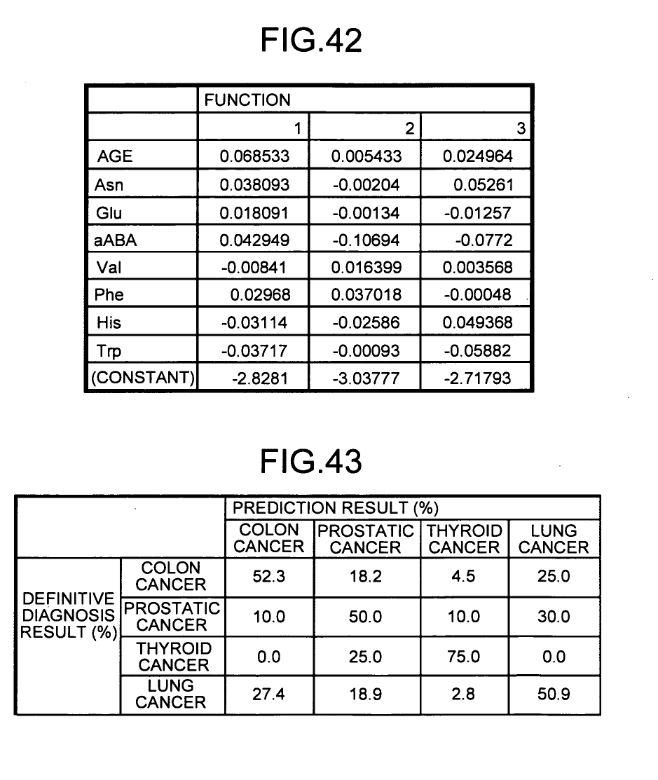

The data of the male colon cancer group, the male prostatic cancer group, the male thyroid cancer group, and the male lung cancer group out of the sample data used in Example 1 is used. Indices to maximize performance to discriminate among four groups of the various cancer groups (colon cancer, prostatic cancer, thyroid cancer, and lung cancer) with respect to cancer are searched by the linear discriminant analysis using the stepwise explanatory variable selecting method, and a linear discriminant group composed of age, Asn, Glu, ABA, Val, Phe, His, and Trp (the coefficients of the age and the amino acid explanatory variables Asn, Glu, ABA, Val, Phe, His, and Trp of each discriminant are presented in FIG. 42) is obtained as an index formula group 5.

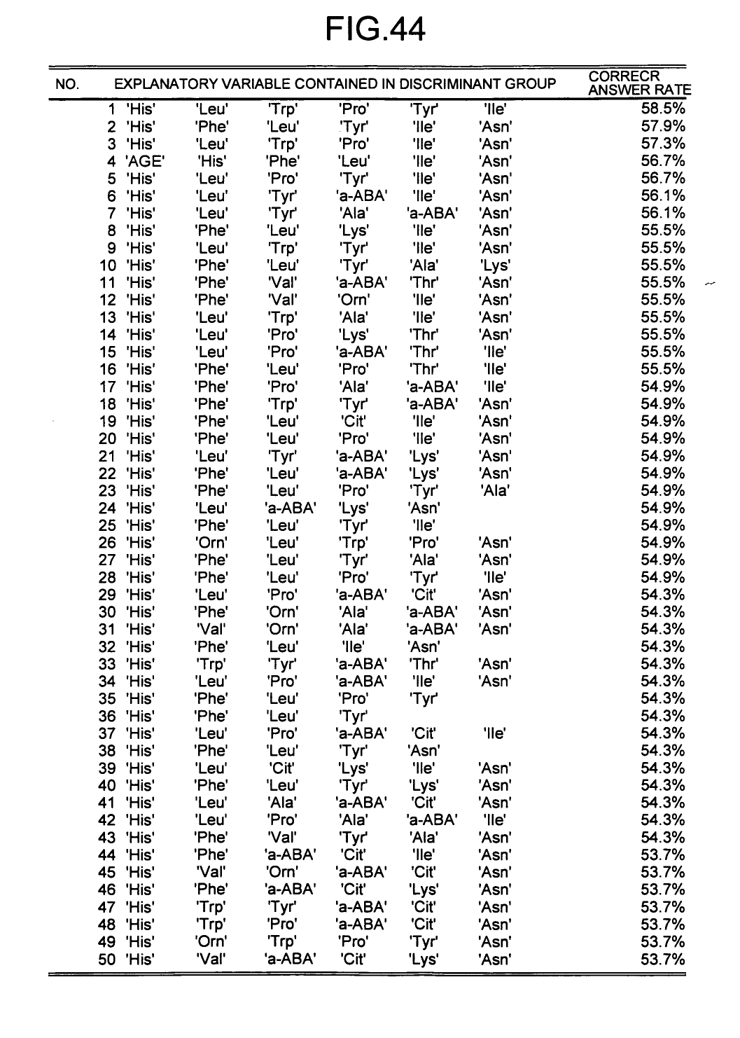

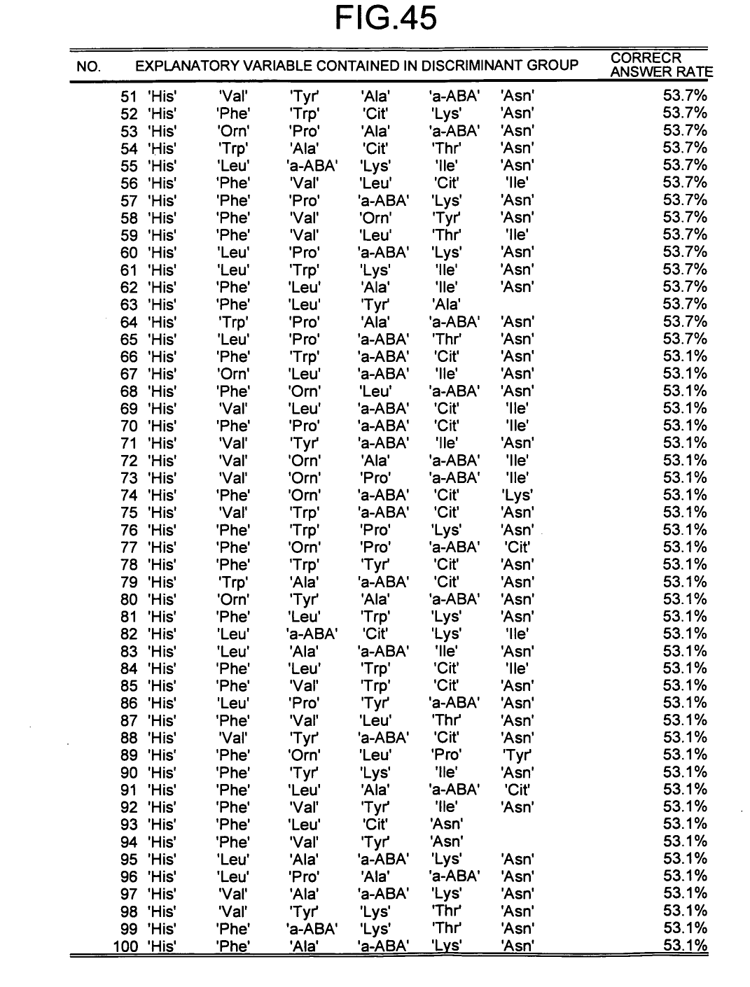

As a result of the evaluation of the diagnosis performance of the various cancers (colon cancer, prostatic cancer, thyroid cancer, and lung cancer) based on the index formula group 5 by correct answer rates of discrimination results, high discrimination ability is demonstrated such that the correct answer rates of the colon cancer, the prostatic cancer, the thyroid cancer, and the lung cancer are 52.3%, 50.0%, 75.0%, and 55.7%, respectively, and the correct answer rate of the total is 51.8% when the prior probability of each is 25.0% (FIG. 43). The value of each coefficient in the discriminant presented in FIG. 42 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 42 is obtained. The list of the explanatory variables contained in the discriminant groups is presented in FIGS. 44 and 45.

### Example 7

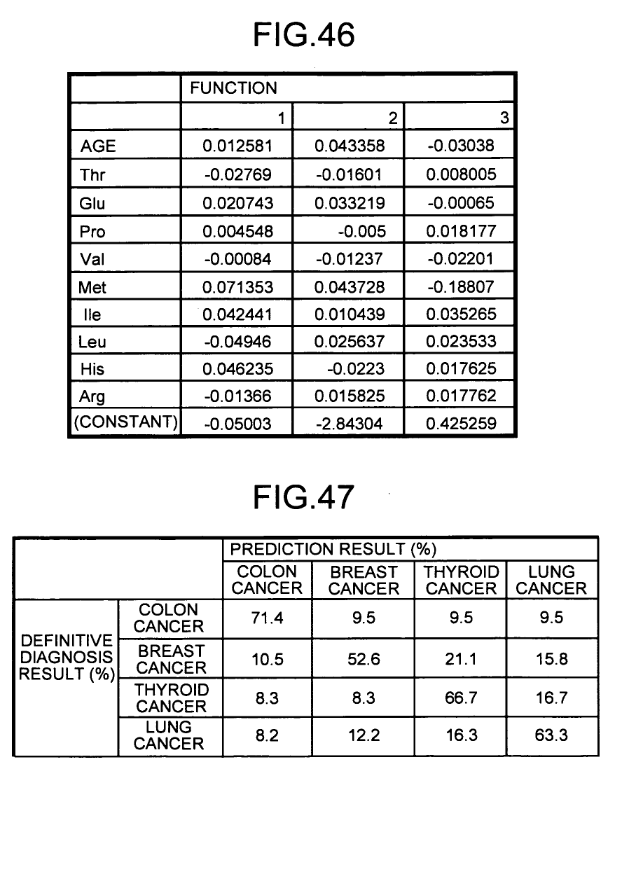

The data of the female colon cancer group, the female breast cancer group, the female thyroid cancer group, and the female lung cancer group out of the sample data used in Example 1 is used. Indices to maximize performance to discriminate among four groups of the various cancer groups (colon cancer, breast cancer, thyroid cancer, and lung cancer) with respect to cancer are searched by the linear discriminant analysis using the stepwise explanatory variable selecting method, and a linear discriminant group composed of age, Thr, Glu, Pro, Val, Met, Ile, Leu, His, and Arg (the coefficients of the age and the amino acid explanatory variables Thr, Glu, Pro, Val, Met, Ile, Leu, His, and Arg of each discriminant are presented in FIG. 46) is obtained as an index formula group 6.

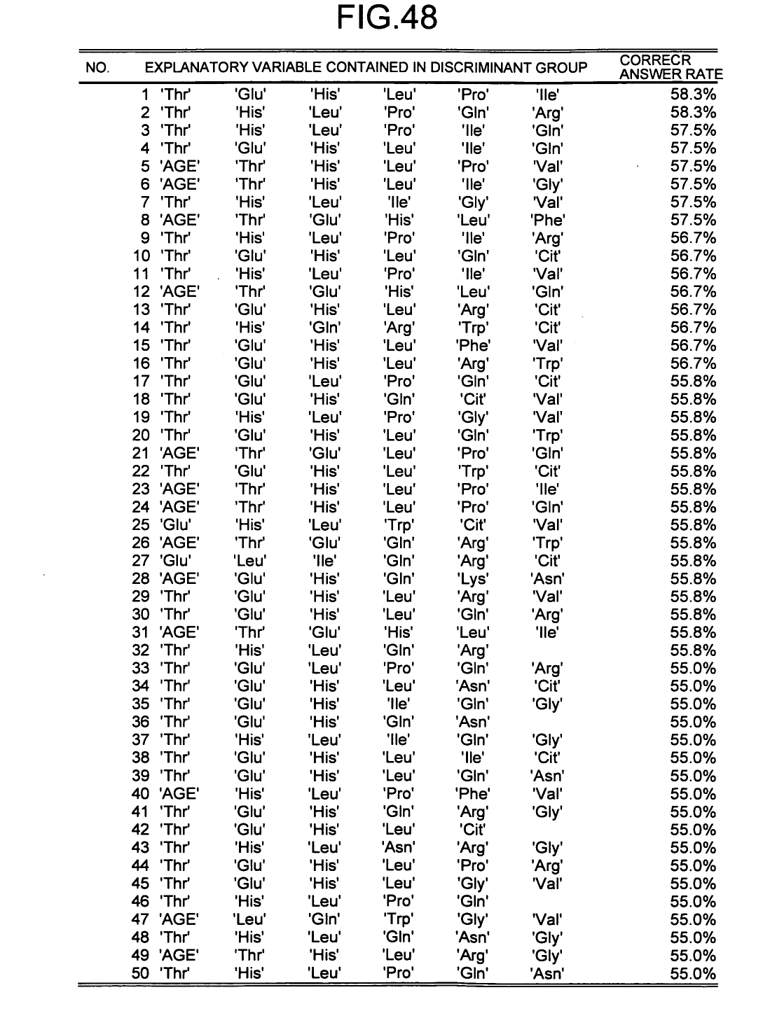

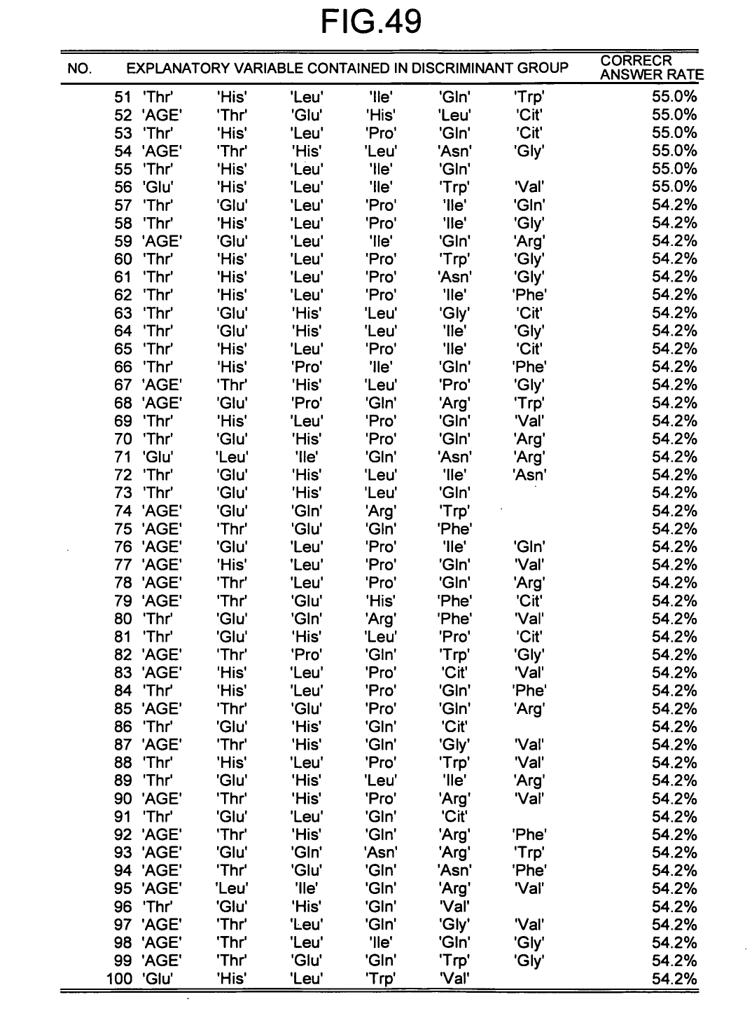

As a result of the evaluation of the diagnosis performance of the various cancers (colon cancer, breast cancer, thyroid cancer, and lung cancer) based on the index formula group 6 by correct answer rates of discrimination results, high discrimination ability is demonstrated such that the correct answer rates of the colon cancer, the breast cancer, the thyroid cancer, and the lung cancer are 71.4%, 52.6%, 66.7%, and 63.3%, respectively, and the correct answer rate of the total is 61.7% when the prior probability of each is 25.0% (FIG. 47). The value of each coefficient in the discriminant presented in FIG. 46 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 46 is obtained. The list of the explanatory variables contained in the discriminant groups is presented in FIGS. 48 and 49.

### Example 8

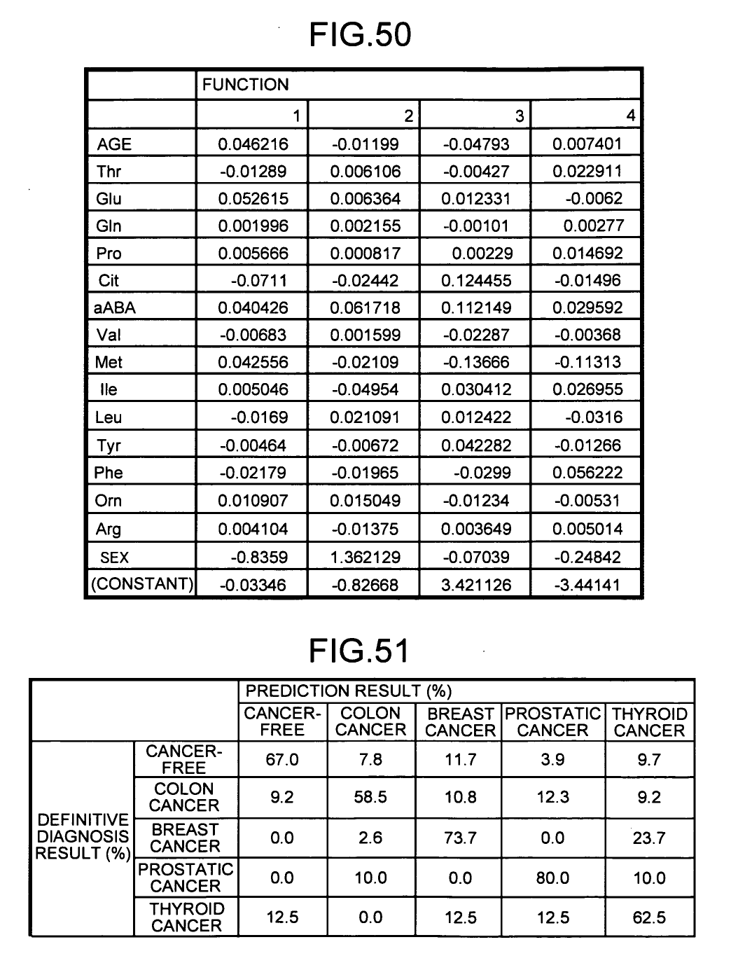

The data of the cancer-free group, the colon cancer group, the breast cancer group, the prostatic cancer group, and the thyroid cancer group out of the sample data used in Example 1 is used. Indices to maximize performance to discriminate among five groups of the various cancer groups (colon cancer, breast cancer, prostatic cancer, and thyroid cancer) and the cancer-free with respect to cancer are searched by the linear discriminant analysis using the stepwise explanatory variable selecting method, and a linear discriminant group composed of age, sex (male=1 and female=2), Thr, Glu, Gln, Pro, Cit, ABA, Val, Met, Ile, Leu, Tyr, Phr, Orn, and Arg (the coefficients of the age, the sex, and the amino acid explanatory variables Thr, Glu, Gln, Pro, Cit, ABA, Val, Met, Ile, Leu, Tyr, Phe, Orn, and Arg of each discriminant are presented in FIG. 50) is obtained as an index formula group 7.

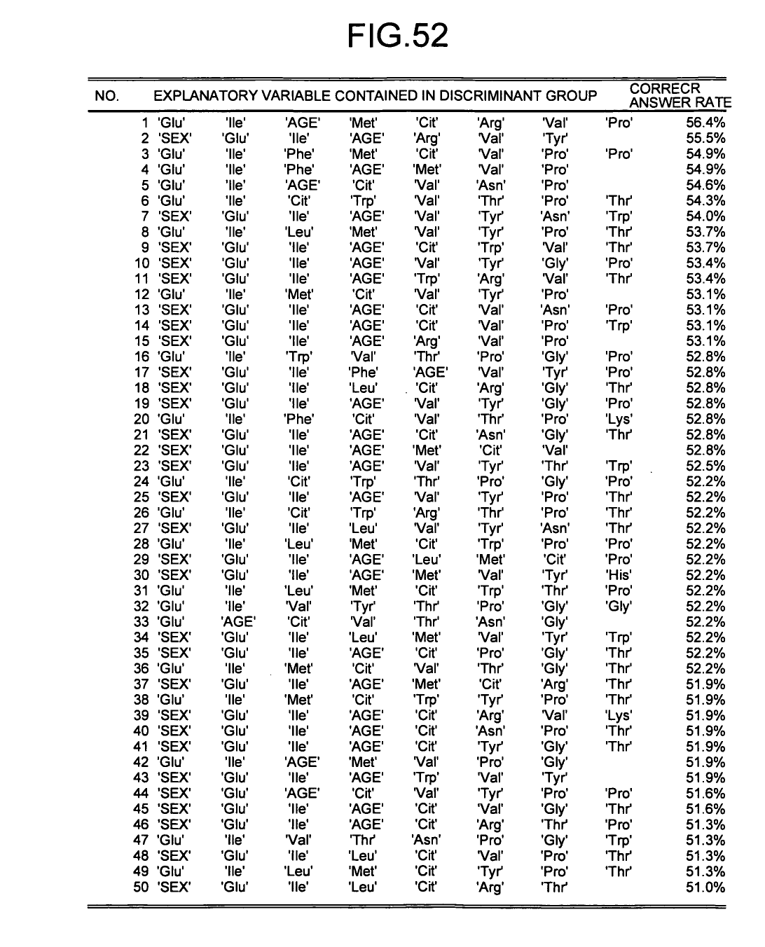

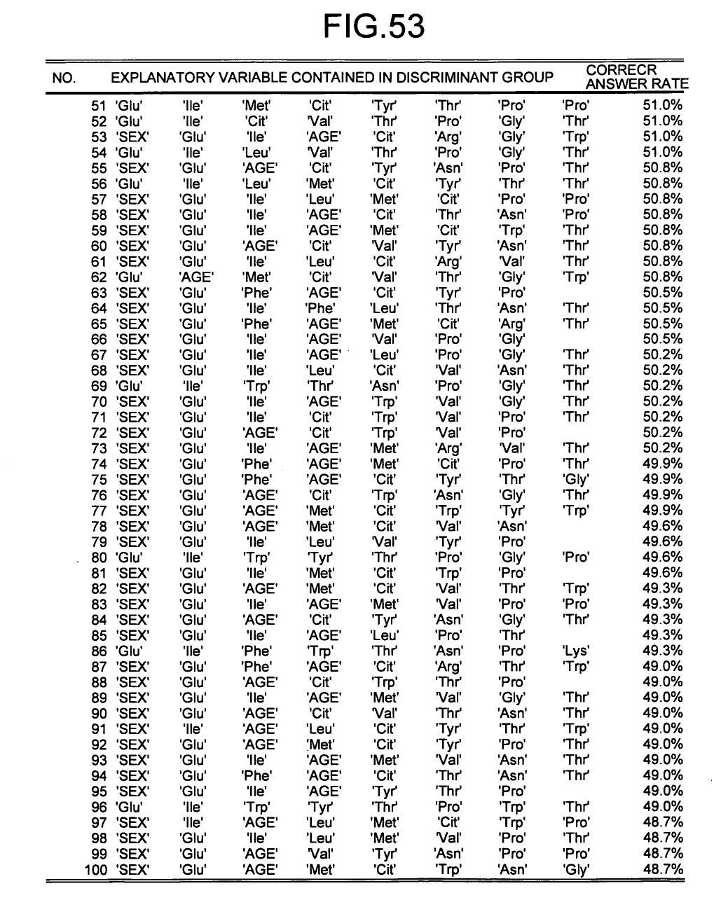

As a result of the evaluation of the diagnosis performance of the various cancers (colon cancer, breast cancer, prostatic cancer, and thyroid cancer) and the cancer-free group based on the index formula group 7 by correct answer rates of discrimination results, high discrimination ability is demonstrated such that the correct answer rates of the cancer-free, the colon cancer, the breast cancer, the prostatic cancer, and the thyroid cancer are 67.0%, 58.5%, 73.7%, 80.0% and 62.5%, respectively, and the correct answer rate of the total is 66.3% when the prior probability of each is 20.0% (FIG. 51). The value of each coefficient in the discriminant presented in FIG. 50 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 50 is obtained. The list of the explanatory variables contained in the discriminant groups is presented in FIGS. 52 and 53.

### Example 9

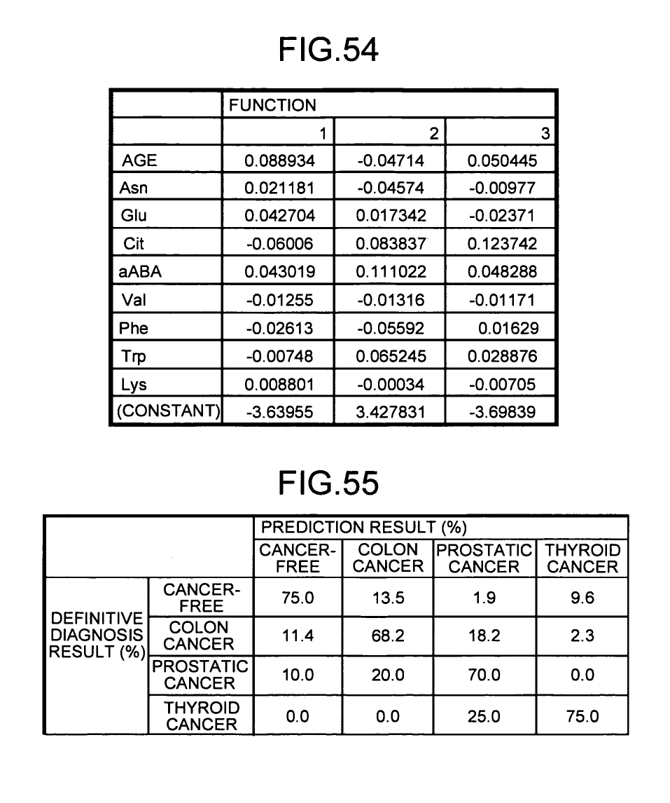

The data of the male cancer-free group, the male colon cancer group, the male prostatic cancer group, and the male thyroid cancer group out of the sample data used in Example 1 is used. Indices to maximize performance to discriminate among four groups of the various cancer groups (colon cancer, prostatic cancer, and thyroid cancer) and the cancer-free group with respect to cancer are searched by the linear discriminant analysis using the stepwise explanatory variable selecting method, and a linear discriminant group composed of age, Asn, Glu, ABA, Val, Phe, His, and Trp (the coefficients of the age and the amino acid explanatory variables Asn, Glu, ABA, Val, Phe, His, and Trp of each discriminant are presented in FIG. 54) is obtained as an index formula group 8.

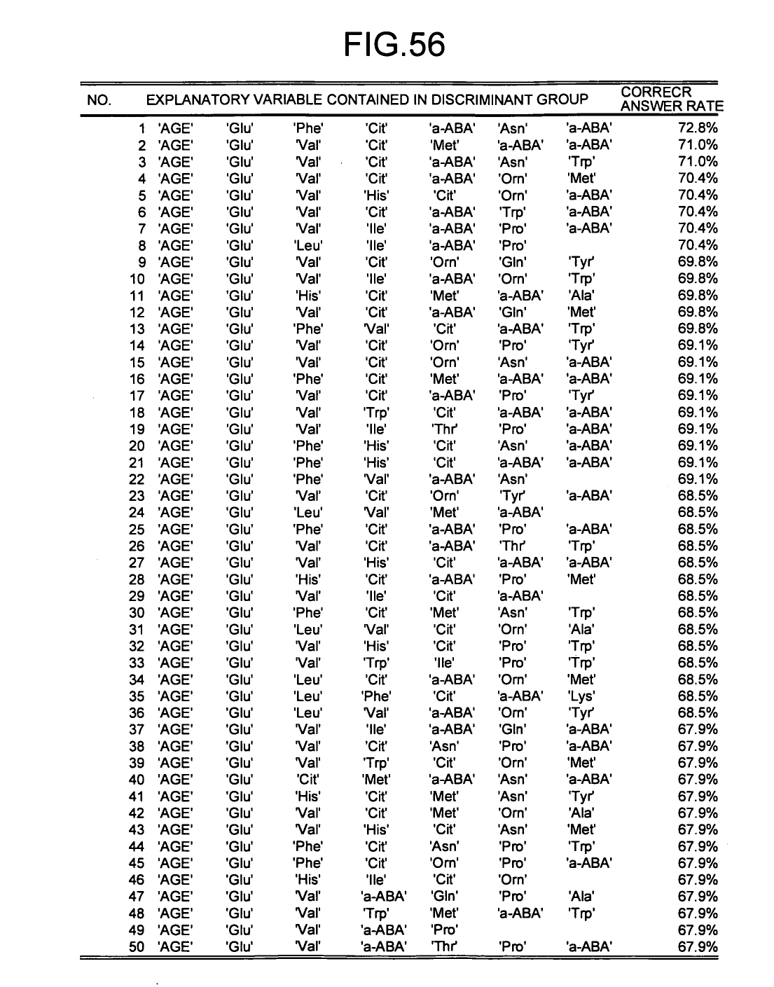

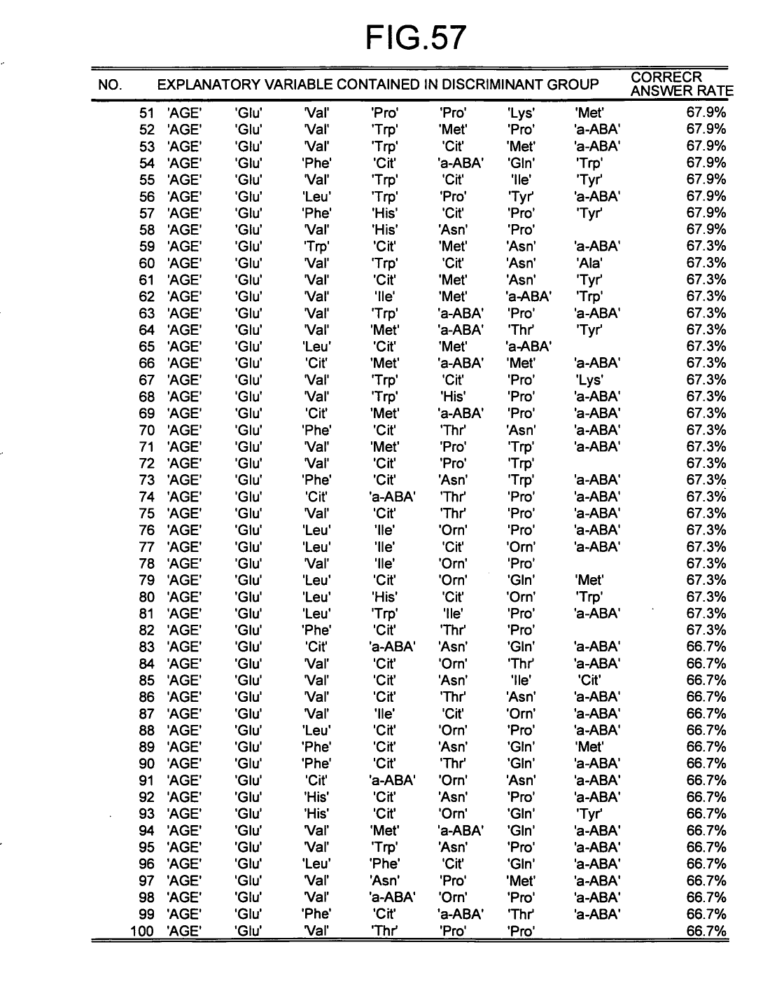

As a result of the evaluation of the diagnosis performance of the various cancers (colon cancer, prostatic cancer, and thyroid cancer) and the cancer-free group based on the index formula group 8 by correct answer rates of discrimination results, high discrimination ability is demonstrated such that the correct answer rates of the cancer-free group, the colon cancer, the prostatic cancer, and the thyroid cancer are 75.0%, 68.2%, 70.0% and 75.0%, respectively, and the correct answer rate of the total is 72.8% when the prior probability of each is 25.0% (FIG. 55). The value of each coefficient in the discriminant presented in FIG. 54 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 54 is obtained. The list of the explanatory variables contained in the discriminant groups is presented in FIGS. 56 and 57.

### Example 10

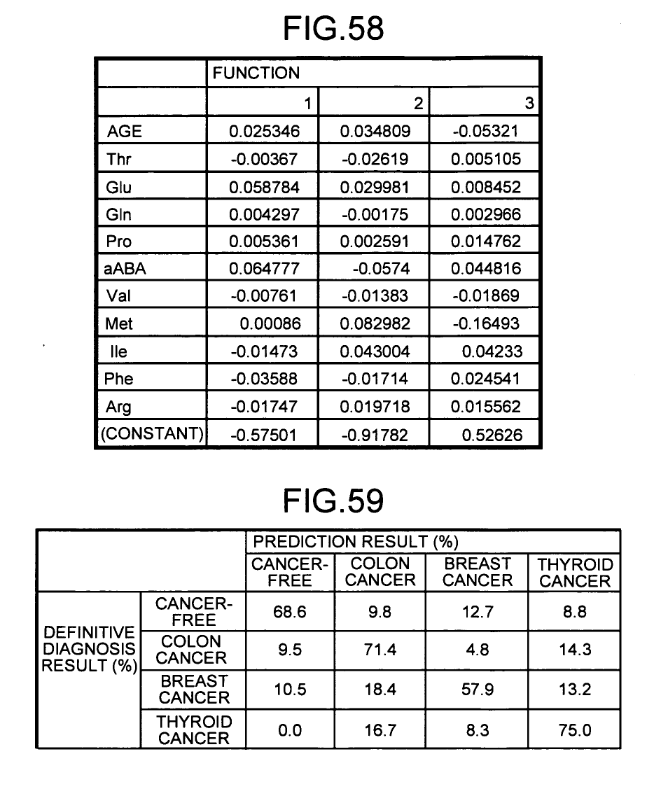

The data of the female cancer-free group, the female colon cancer group, the female breast cancer group, and the female thyroid cancer group out of the sample data used in Example 1 is used. Indices to maximize performance to discriminate among four groups of the various cancer groups (colon cancer, breast cancer, and thyroid cancer) and the cancer-free with respect to cancer are searched by the linear discriminant analysis using the stepwise explanatory variable selecting method, and a linear discriminant group composed of age, Thr, Glu, Gln, Pro, ABA, Val, Met, Ile, Phe, and Arg (the coefficients of the age and the amino acid explanatory variables Thr, Glu, Gln, Pro, ABA, Val, Met, Ile, Phe, and Arg of each discriminant are presented in FIG. 58) is obtained as an index formula group 9.

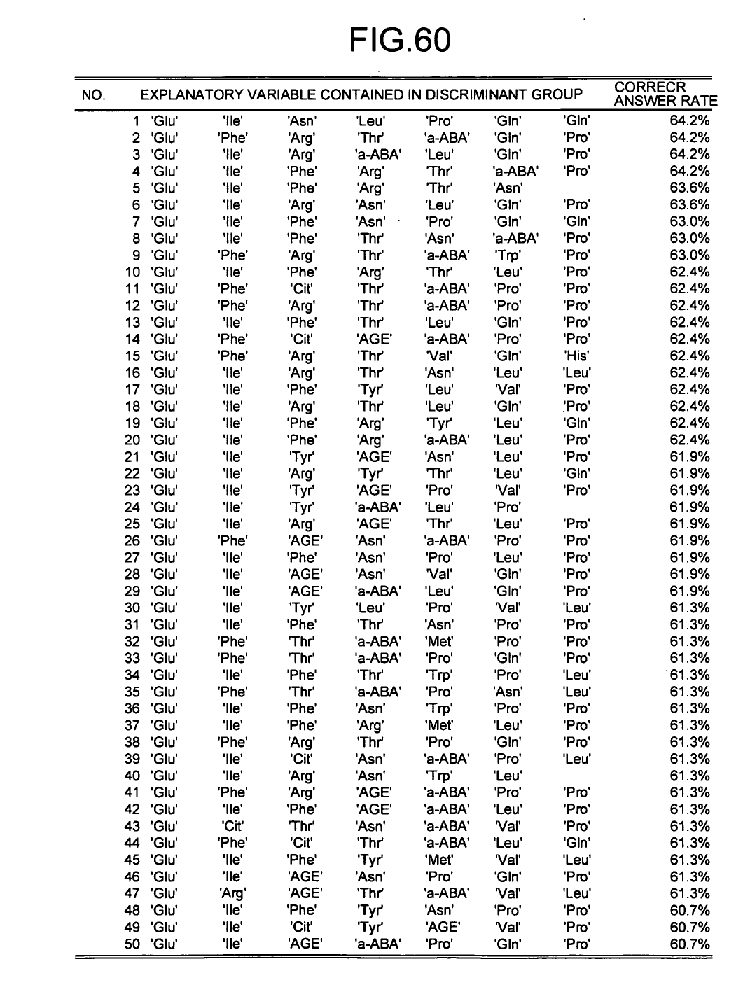

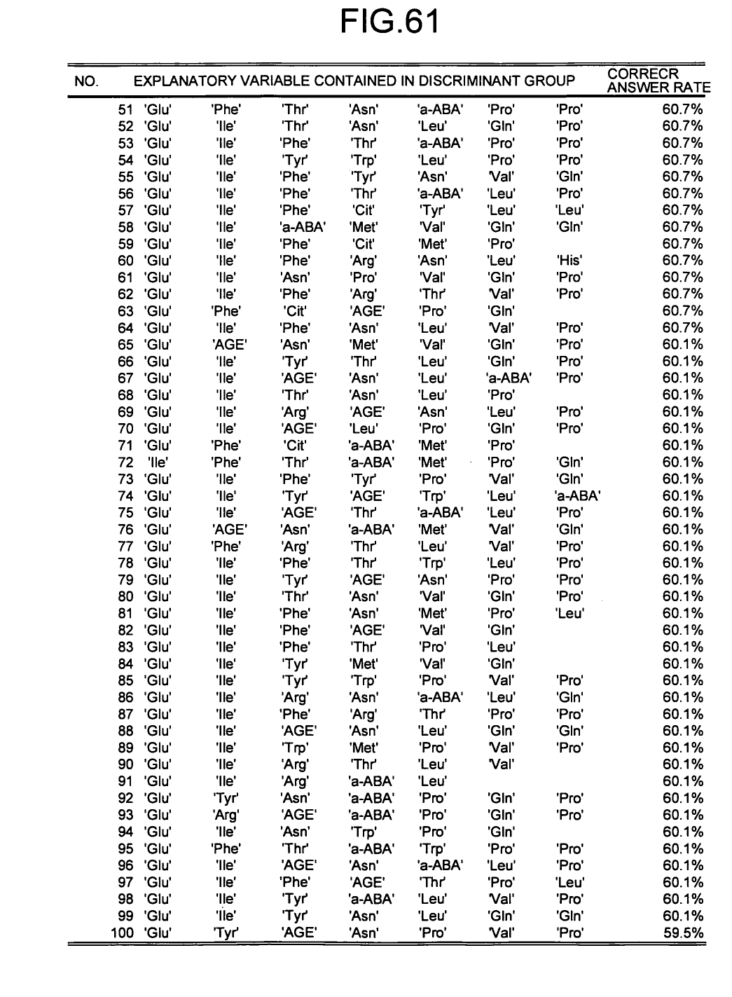

As a result of the evaluation of the diagnosis performance of the various cancers (colon cancer, breast cancer, and thyroid cancer) and the cancer-free group based on the index formula group 9 by correct answer rates of discrimination results, high discrimination ability is demonstrated such that the correct answer rates of the cancer-free group, the colon cancer, the breast cancer, and the thyroid cancer are 68.6%, 71.4%, 57.9%, and 75.0%, respectively, and the correct answer rate of the total is 67.1% when the prior probability of each is 25.0% (FIG. 59). The value of each coefficient in the discriminant presented in FIG. 58 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 58 is obtained. The list of the explanatory variables contained in the discriminant groups is presented in FIGS. 60 and 61.

### Example 11

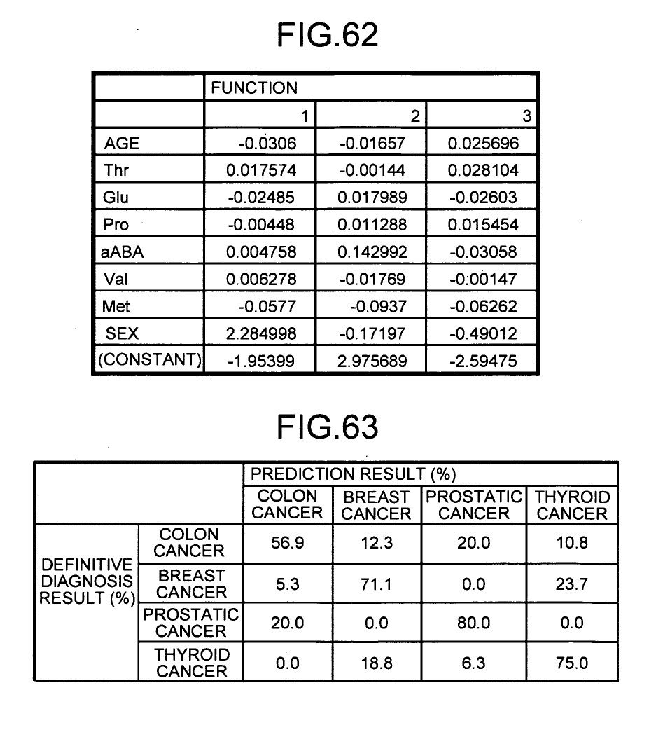

The data of the colon cancer group, the breast cancer group, the prostatic cancer group, and the thyroid cancer group out of the sample data used in Example 1 is used. Indices to maximize performance to discriminate among four groups of the various cancer groups (colon cancer, breast cancer, prostatic cancer, and thyroid cancer) with respect to cancer are searched by the linear discriminant analysis using the stepwise explanatory variable selecting method, and a linear discriminant group composed of age, sex (male=1 and female=2), Thr, Glu, Pro, ABA, Val, and Met (the coefficients of the age, the sex, and the amino acid explanatory variables Thr, Glu, Pro, ABA, Val, and Met of each discriminant are presented in FIG. 62) is obtained as an index formula group 10.

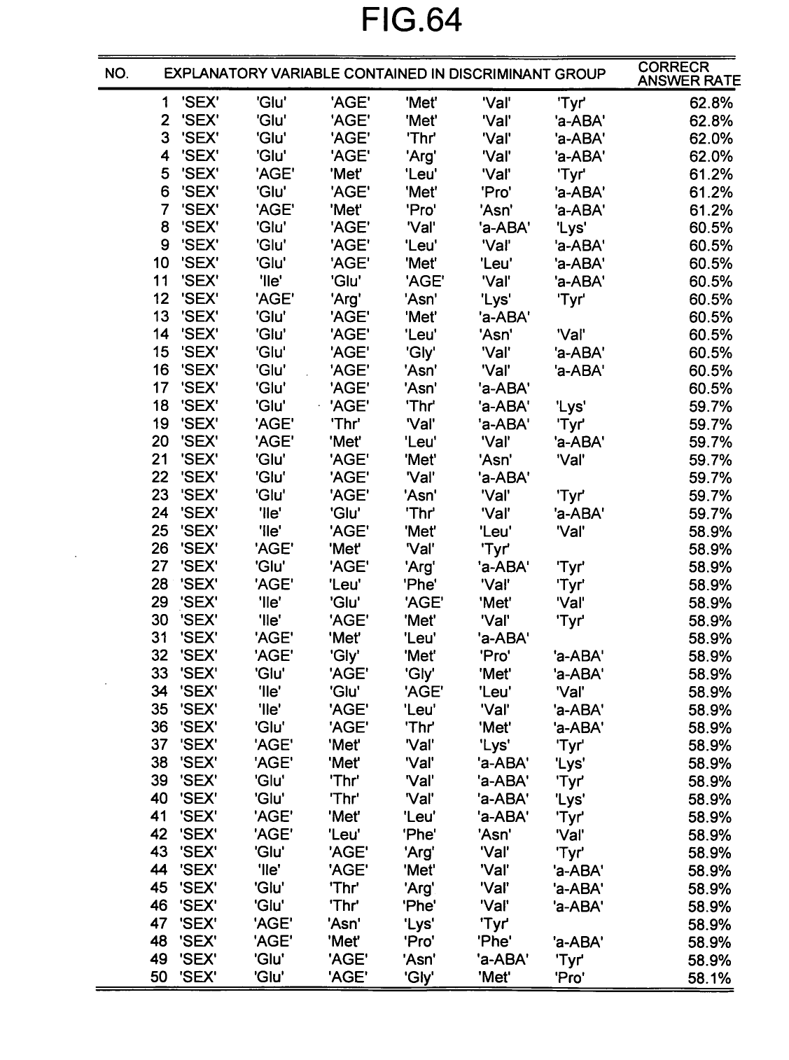

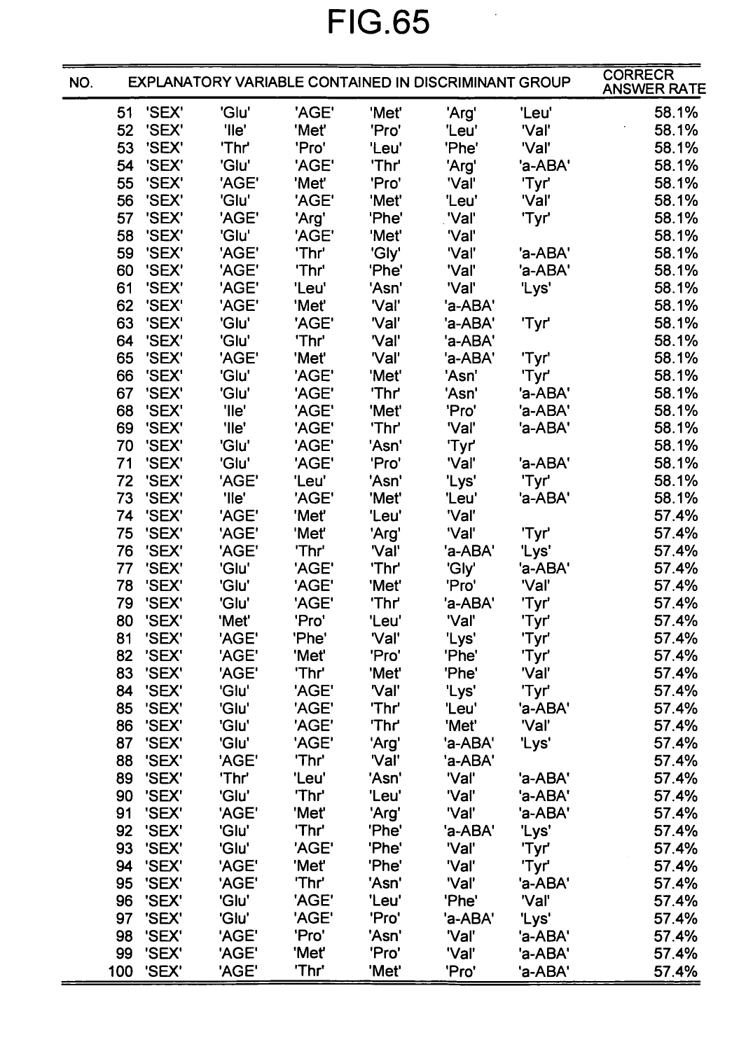

As a result of the evaluation of the diagnosis performance of the various cancers (colon cancer, breast cancer, prostatic cancer, and thyroid cancer) based on the index formula group 10 by correct answer rates of discrimination results, high discrimination ability is demonstrated such that the correct answer rates of the colon cancer, the breast cancer, the prostatic cancer, and the thyroid cancer are 56.9%, 71.1%, 80.0%, and 75.0%, respectively, and the correct answer rate of the total is 65.1% when the prior probability of each is 25.0% (FIG. 63). The value of each coefficient in the discriminant presented in FIG. 62 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 62 is obtained. The list of the explanatory variables contained in the discriminant groups is presented in FIGS. 64 and 65.

### Example 12

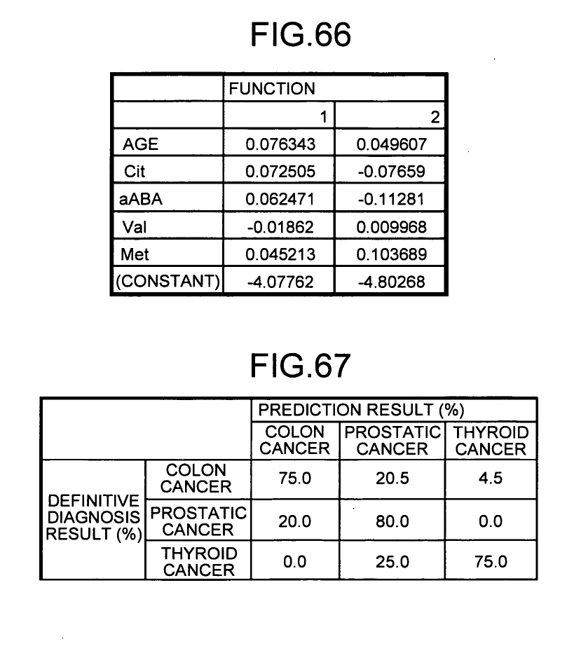

The data of the male colon cancer group, the male prostatic cancer group, and the male thyroid cancer group out of the sample data used in Example 1 is used. Indices to maximize performance to discriminate among three groups of the various cancer groups (colon cancer, prostatic cancer, and thyroid cancer) with respect to cancer are searched by the linear discriminant analysis using the stepwise explanatory variable selecting method, and a linear discriminant group composed of age, Cit, ABA, Val, and Met (the coefficients of the age and the amino acid explanatory variables Cit, ABA, Val, and Met of each discriminant are presented in FIG. 66) is obtained as an index formula group 11.

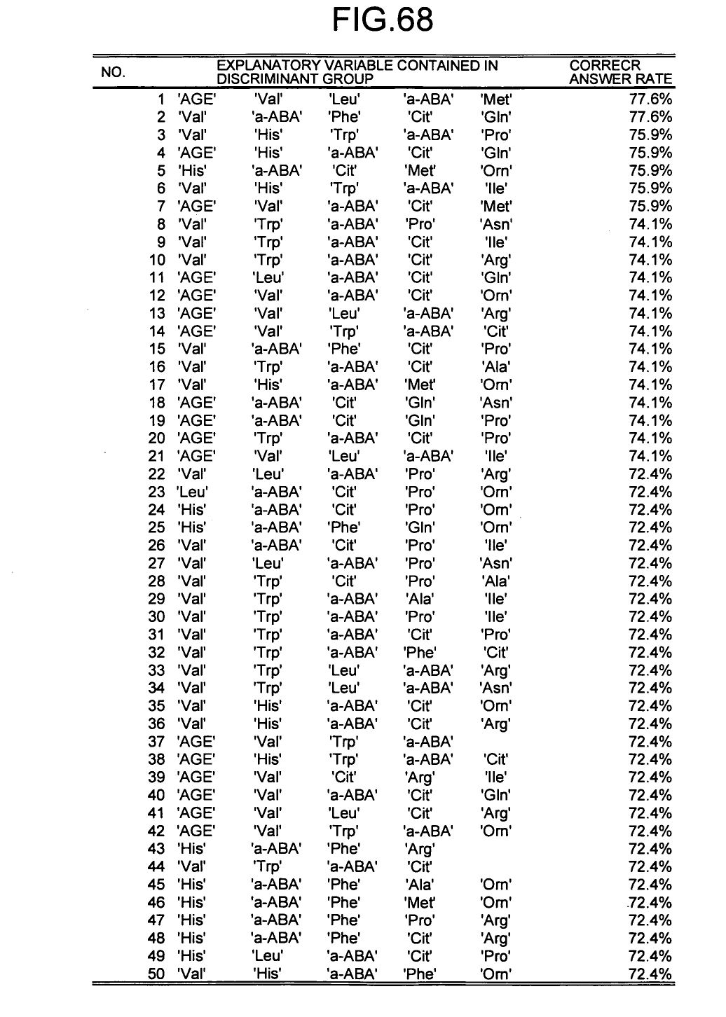

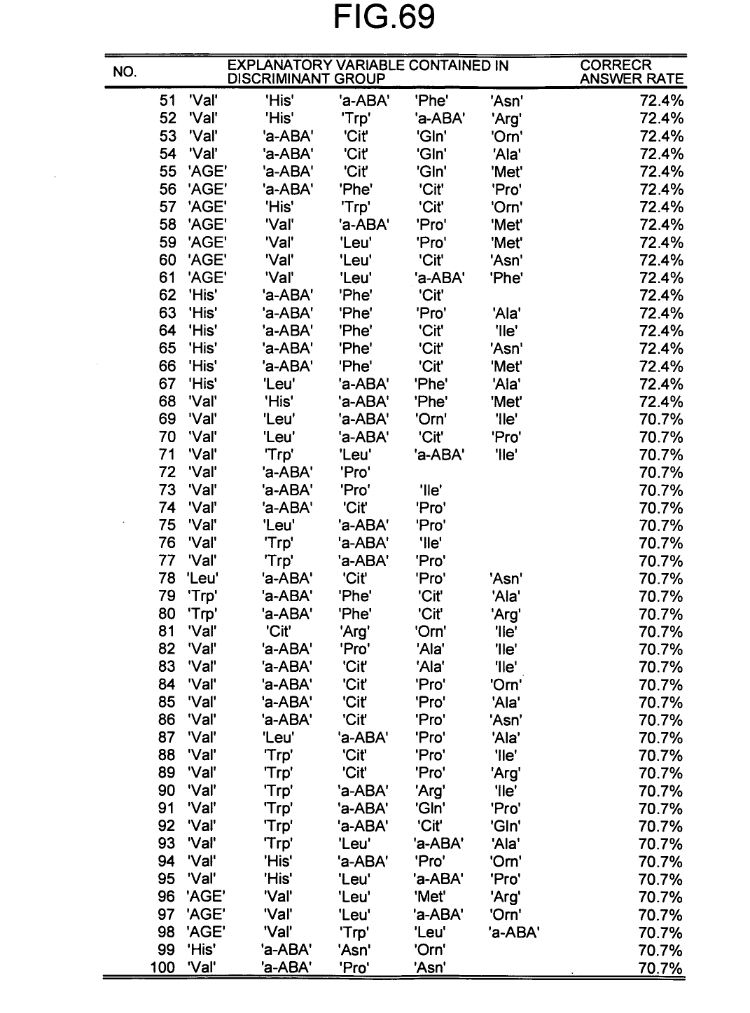

As a result of the evaluation of the diagnosis performance of the various cancers (colon cancer, prostatic cancer, and thyroid cancer) based on the index formula group 11 by correct answer rates of discrimination results, high discrimination ability is demonstrated such that the correct answer rates of the colon cancer, the prostatic cancer, and the thyroid cancer are 75.0%, 80.0%, and 75.0%, respectively, and the correct answer rate of the total is 75.9% when the prior probability of each is 33.3% (FIG. 67). The value of each coefficient in the discriminant presented in FIG. 66 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 66 is obtained. The list of the explanatory variables contained in the discriminant groups is presented in FIGS. 68 and 69.

### Example 13

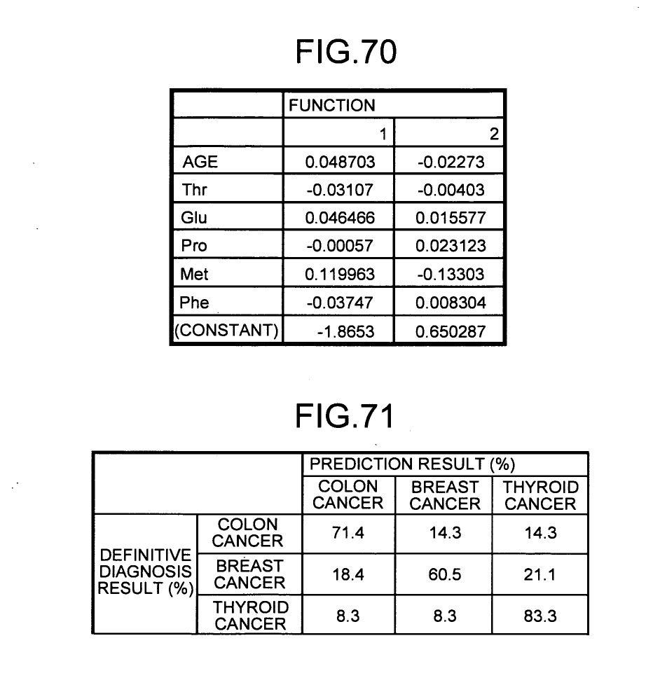

The data of the female colon cancer group, the female breast cancer group, and the female thyroid cancer group out of the sample data used in Example 1 is used. Indices to maximize performance to discriminate among three groups of the various cancer groups (colon cancer, breast cancer, and thyroid cancer) with respect to cancer are searched by the linear discriminant analysis using the stepwise explanatory variable selecting method, and a linear discriminant group composed of age, Thr, Glu, Pro, Met, and Phe (the coefficients of the age and the amino acid explanatory variables Thr, Glu, Pro, Met, and Phe of each discriminant are presented in FIG. 70) is obtained as an index formula group 12.

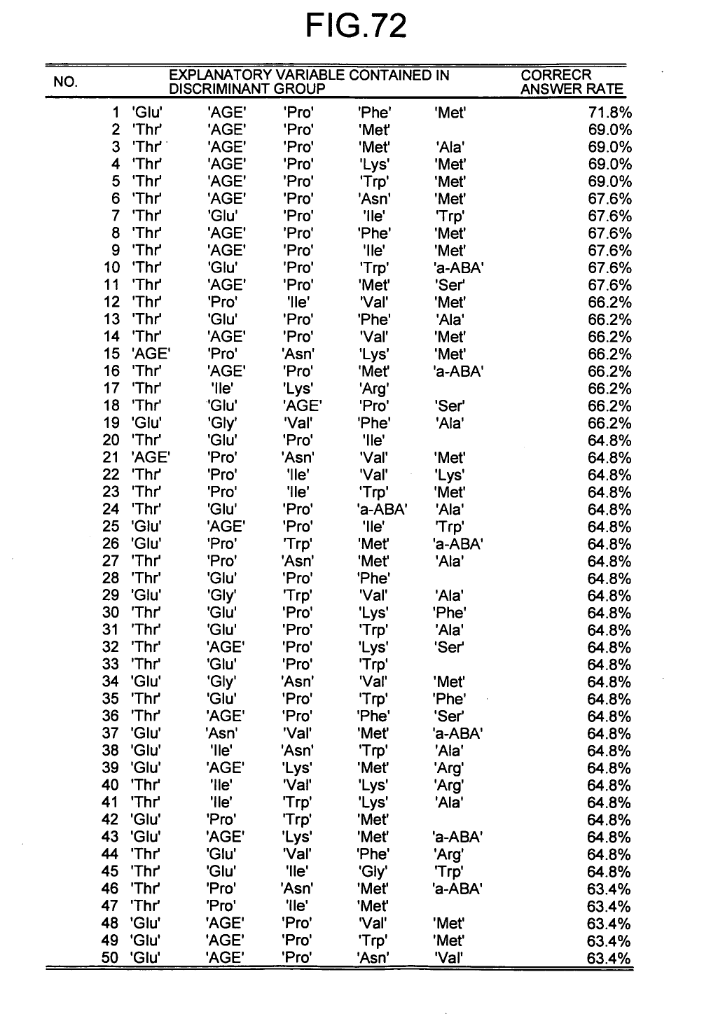

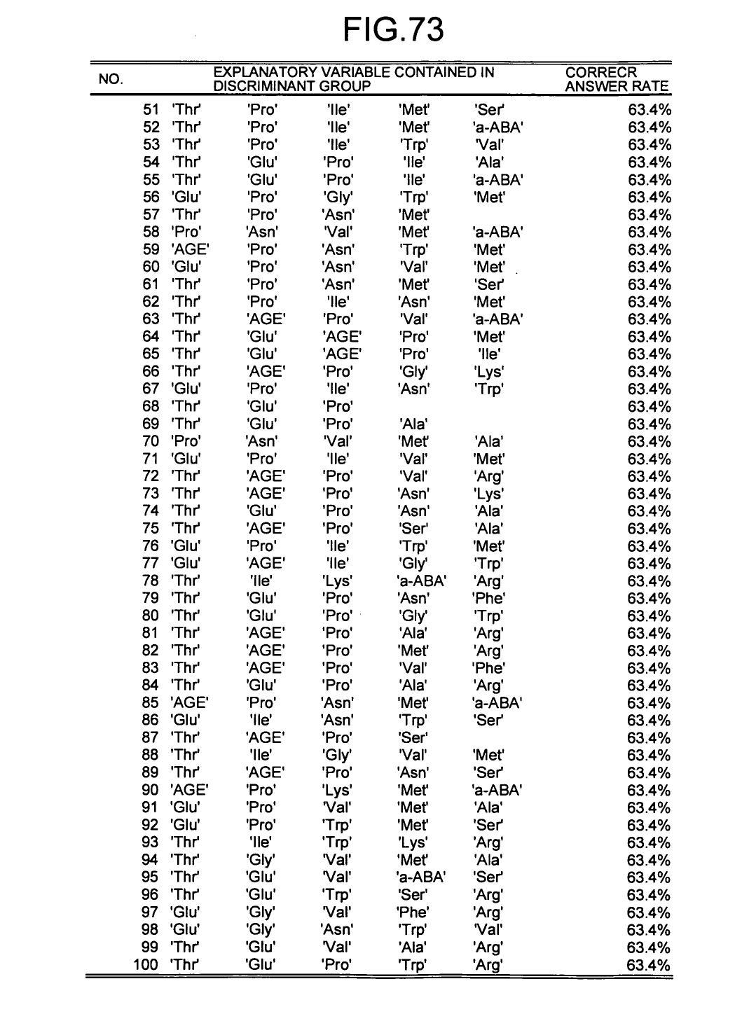

As a result of the evaluation of the diagnosis performance of the various cancers (colon cancer, breast cancer, and thyroid cancer) based on the index formula group 12 by correct answer rates of discrimination results, high discrimination ability is demonstrated such that the correct answer rates of the colon cancer, the breast cancer, and the thyroid cancer are 71.4%, 60.5%, and 83.3%, respectively, and the correct answer rate of the total is 67.6% when the prior probability of each is 33.3% (FIG. 71). The value of each coefficient in the discriminant presented in FIG. 70 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 70 is obtained. The list of the explanatory variables contained in the discriminant groups is presented in FIGS. 72 and 73.

### Example 14

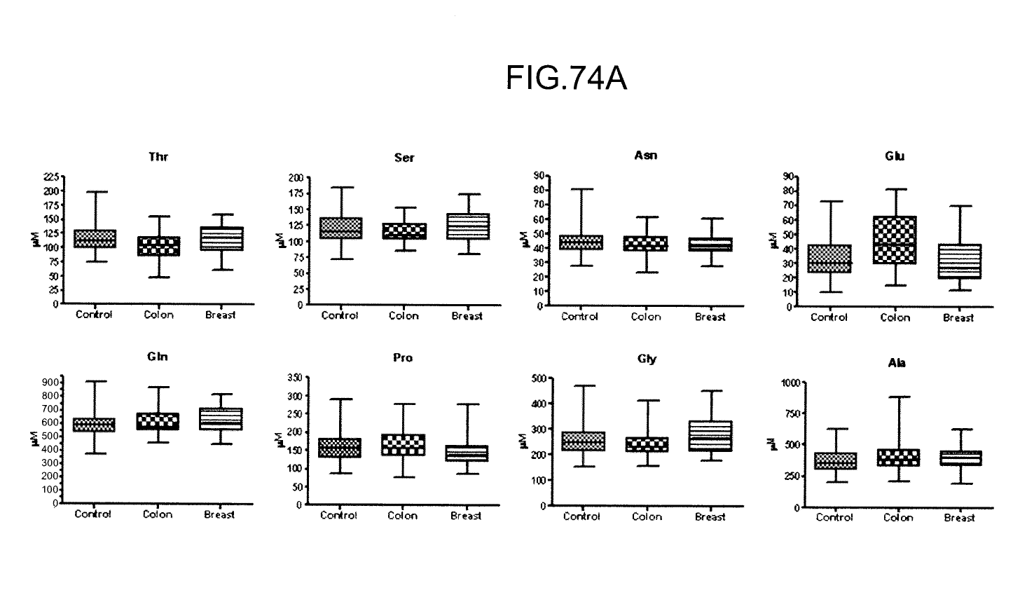

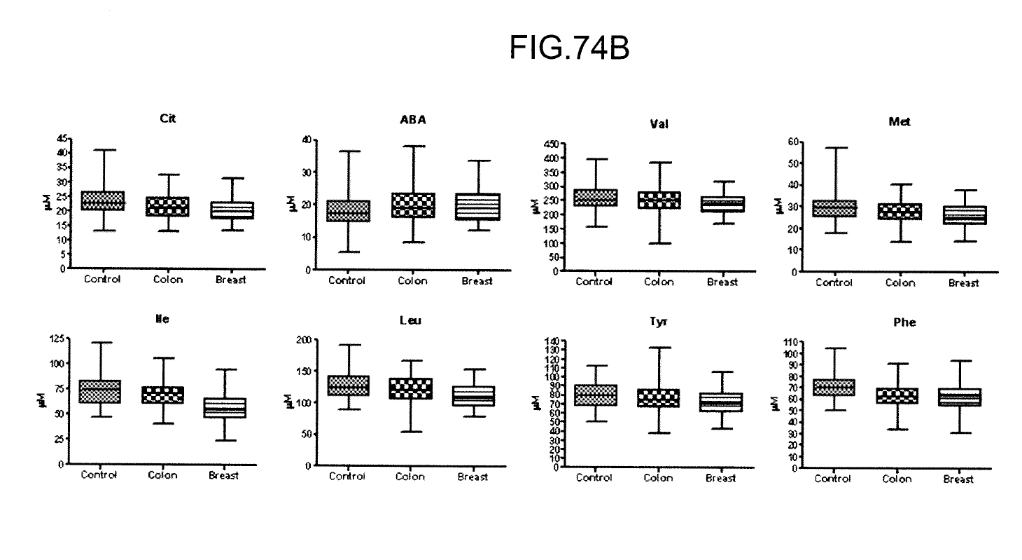

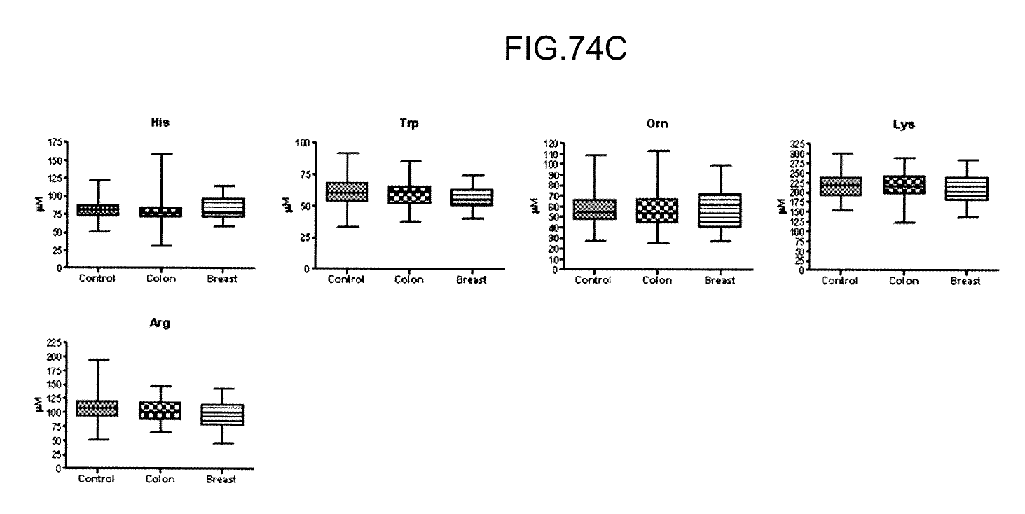

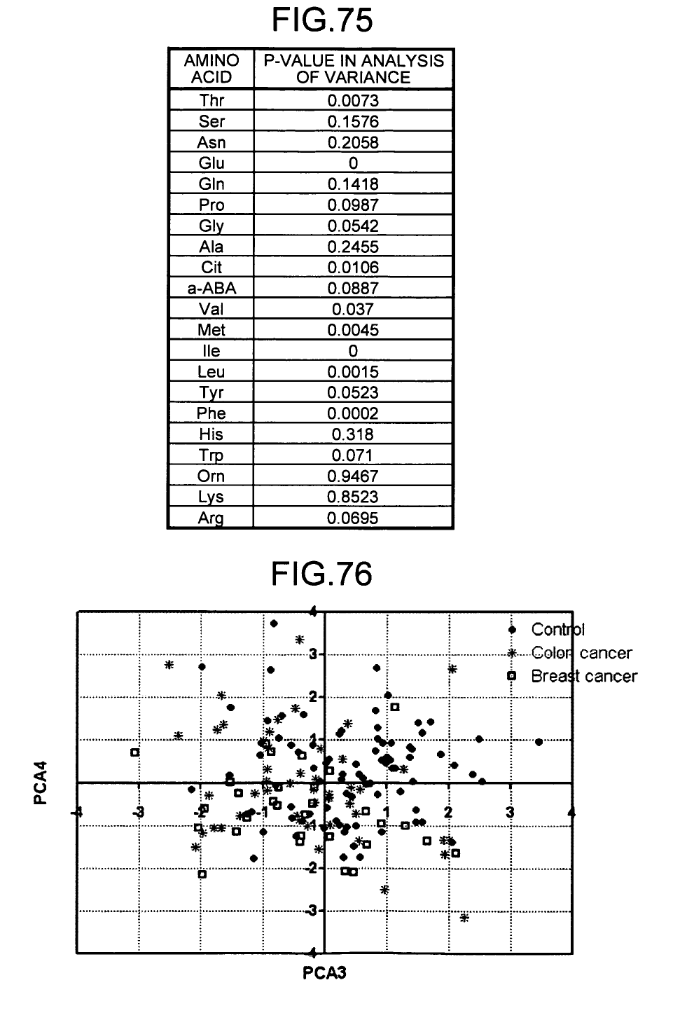

Amino acid concentration in blood is measured by the amino acid analysis method in blood samples of various cancer patient groups with definite diagnosis of colon cancer or breast cancer and blood samples of cancer-free group. The unit of the amino acid concentration is nmol/ml. FIG. 74 is boxplots showing the distribution of the amino acid explanatory variables of various cancer patients and cancer-free subjects. In FIG. 74, the horizontal axis indicates the cancer-free group and the various cancer groups, and ABA in the figure represents α-ABA (α-aminobutyric acid). Further, evaluation using one-way analysis of variance is carried out with respect to discrimination among the various cancer groups and the cancer-free group by each amino acid explanatory variable, and p-values of the amino acid explanatory variables Thr, Glu, Cit, Val, Met, Ile, Leu, and Phe are smaller than 0.05 (FIG. 75). As a result, it is proved that the amino acid explanatory variables Thr, Glu, Cit, Val, Met, Ile, Leu, and Phe have an ability to discriminate among three groups of the colon cancer group, the breast cancer group, and the cancer-free group.

### Example 15

The sample data used in Example 14 is used. Criteria of the concentration data of the amino acid explanatory variables are established. That is to say, a value obtained by performing conversion “(the concentration data of each amino acid explanatory variable−the average of the concentration of each amino acid explanatory variable)/the standard deviation of the concentration of each amino acid explanatory variable” is obtained. When extracting a principal component of which eigenvalue is larger than 1 by performing principal component analysis using the obtained criteria data, first to fifth principal components are obtained. As a result of plotting the third principal component and the fourth principal component on an x-axis and y-axis, respectively, it is proved that the cancer-free group and the colon cancer group, the cancer-free group and the breast cancer group, the cancer-free group and (the colon cancer group+the breast cancer group), and the colon cancer group and the breast cancer group are separated from each other (FIG. 76), and it is proved that the colon cancer group, the breast cancer group, and the cancer-free group can be discriminated from one another using the amino acid explanatory variables.

### Example 16

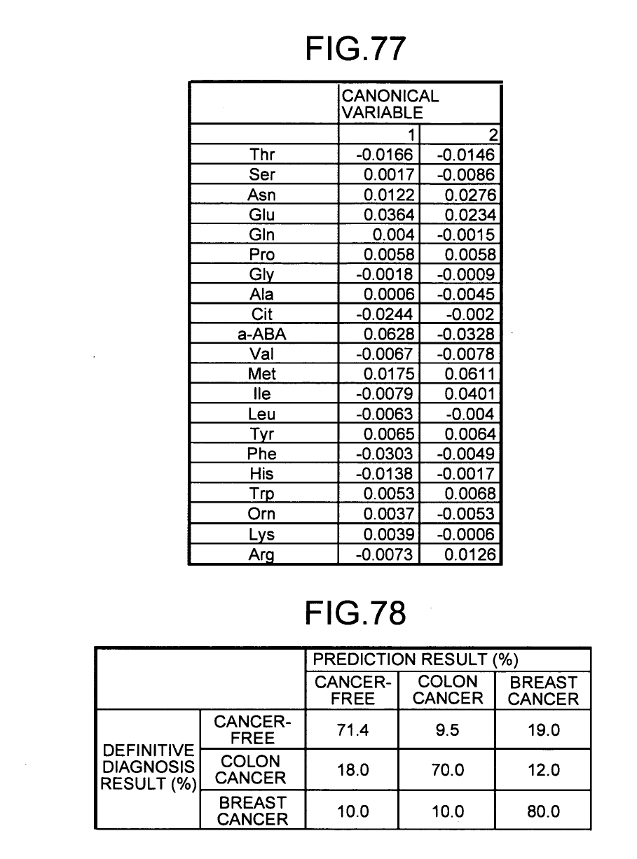

The sample data used in Example 14 is used. As a result of canonical correlation analysis using the total concentration data of the amino acid explanatory variables and numerical category data of each case (colon cancer=1 and breast cancer and cancer-free=0, and breast cancer=1 and colon cancer and cancer-free=0), two index formula groups 13 composed of synthetic explanatory variables of the concentration data of the amino acid explanatory variables are obtained. The coefficient of each amino acid explanatory variable composing the obtained canonical variable group is presented in FIG. 77. Further, as a result of the evaluation of the diagnosis performance of the colon cancer, the breast cancer, and the cancer-free group by correct answer rates of discrimination results by performing the discriminant analysis by the Mahalanobis' generalized distance using the obtained index formula group 13, high discrimination ability is demonstrated such that the correct answer rates of the cancer-free, the colon cancer, and the breast cancer are 71.4%, 70.0%, and 80.0%, respectively, and the correct answer rate of the total is 72.6% when the prior probability of each is 33.3% (FIG. 78). The value of each coefficient in the discriminant presented in FIG. 77 may be a value obtained by multiplying the same by a real number, and the value of the constant term may be a value obtained by carrying out the addition, subtraction, multiplication or division of an arbitrary real constant to the same.

### Example 17

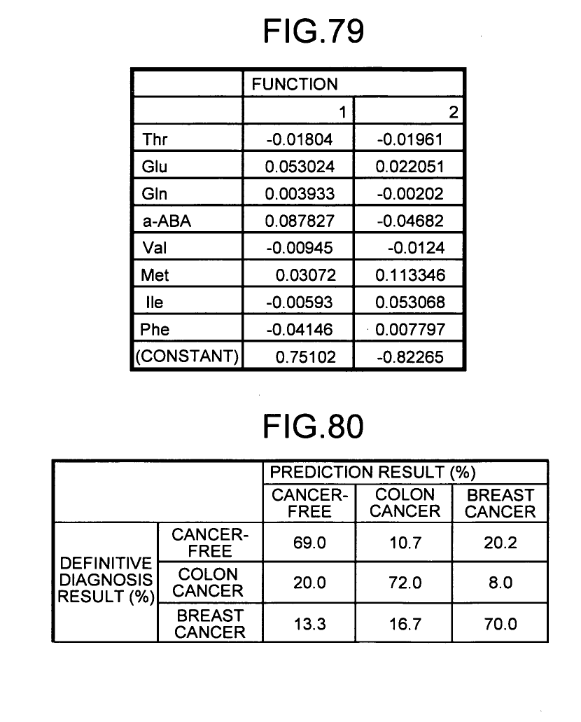

The sample data used in Example 14 is used. Indices to maximize performance to discriminate three groups of the colon cancer group, the breast cancer group, and the cancer-free group with respect to cancer are searched by the linear discriminant analysis using the stepwise explanatory variable selecting method, and a linear discriminant group composed of Thr, Glu, Gln, a-ABA, Val, Met, Ile, and Phe (the coefficients of the amino acid explanatory variables Thr, Glu, Gln, a-ABA, Val, Met, Ile, and Phe of each discriminant are presented in FIG. 79) is obtained as an index formula group 14.

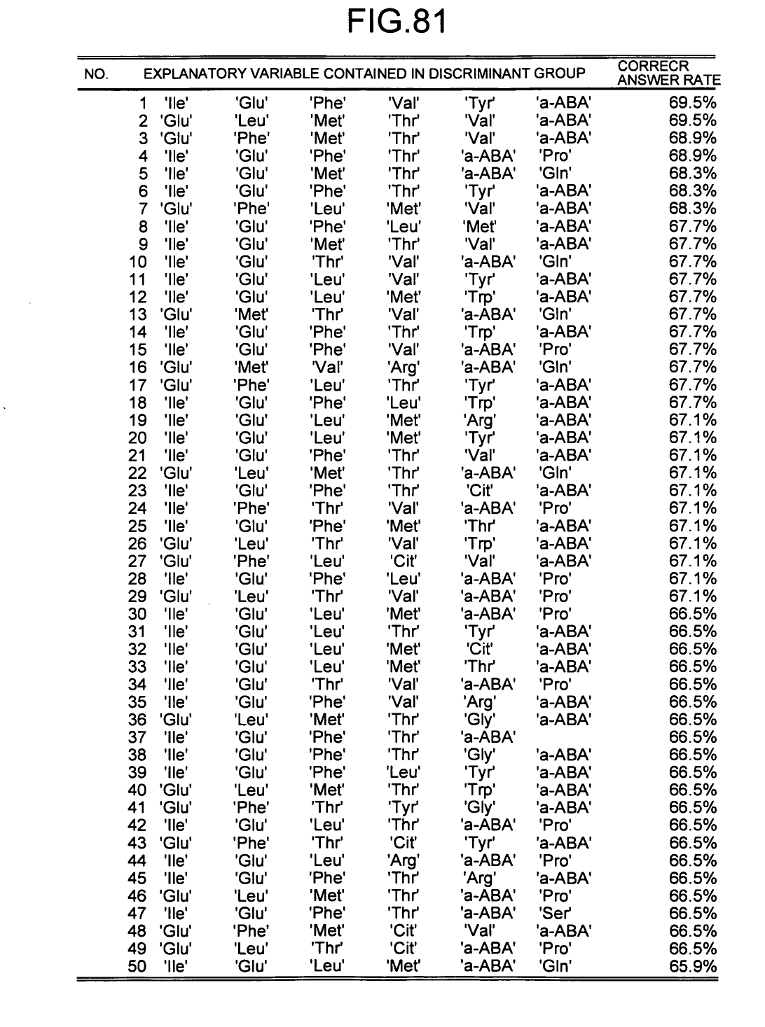

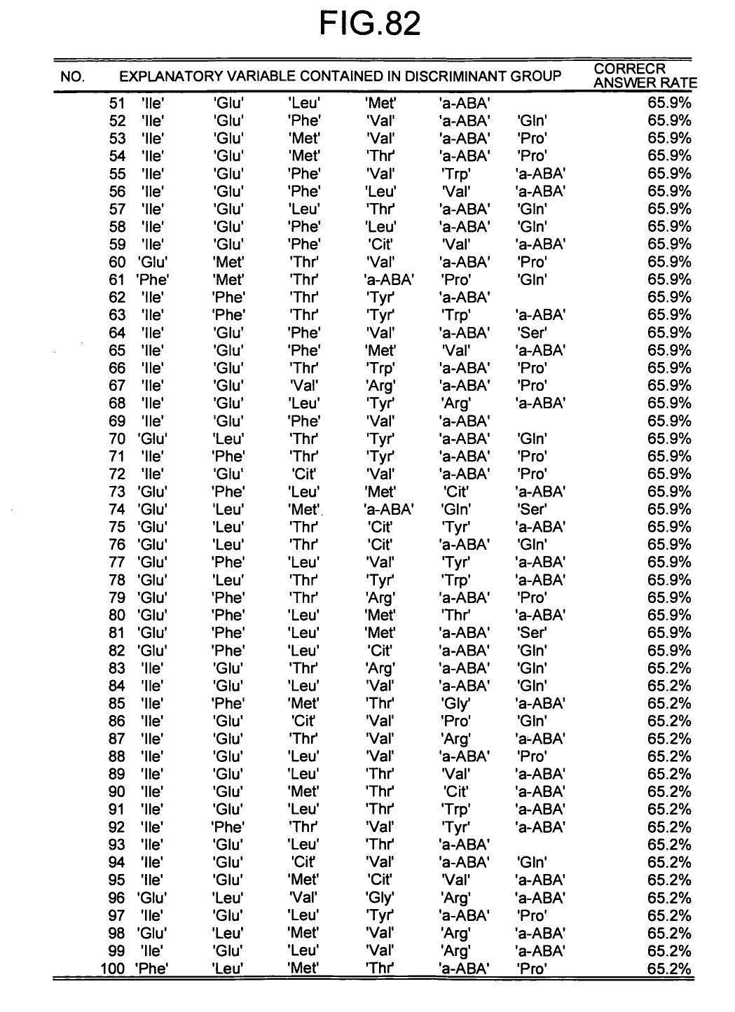

As a result of the evaluation of the diagnosis performance of the colon cancer, the breast cancer, and the cancer-free group based on the index formula group 14 by correct answer rates of discrimination results, high discrimination ability is demonstrated such that the correct answer rates of the cancer-free, the colon cancer, and the breast cancer are 69.0%, 72.0%, and 70.0%, respectively, and the correct answer rate of the total is 70.1% when the prior probability of each is 33.3% (FIG. 80). The value of each coefficient in the discriminant presented in FIG. 79 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 79 is obtained. The list of the explanatory variables contained in the discriminant groups is presented in FIGS. 81 and 82.

### Example 18

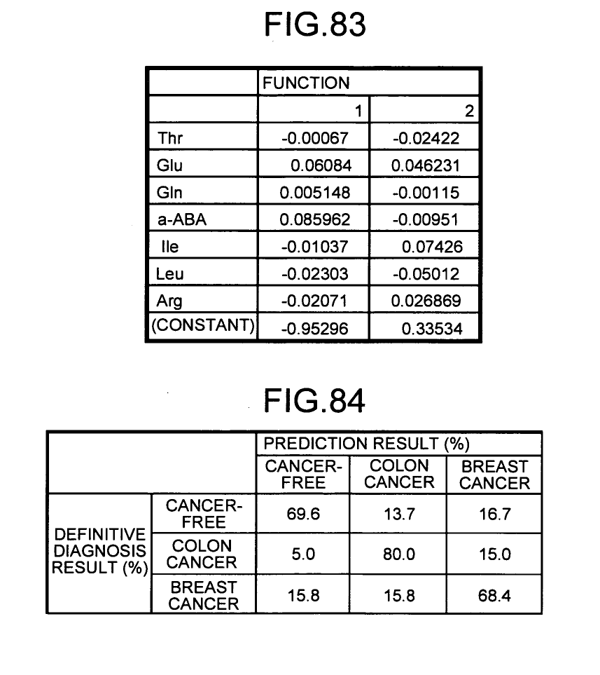

The female data out of the sample data used in Example 14 is used. Indices to maximize performance to discriminate among three groups of the colon cancer group, the breast cancer group, and the cancer-free group with respect to cancer are searched by the linear discriminant analysis using the stepwise explanatory variable selecting method, and a linear discriminant group composed of Thr, Glu, Gln, ABA, Ile, Leu, and Arg (the coefficients of the amino acid explanatory variables Thr, Glu, Gln, ABA, Ile, Leu, and Arg of each discriminant are presented in FIG. 83) is obtained as an index formula group 15.

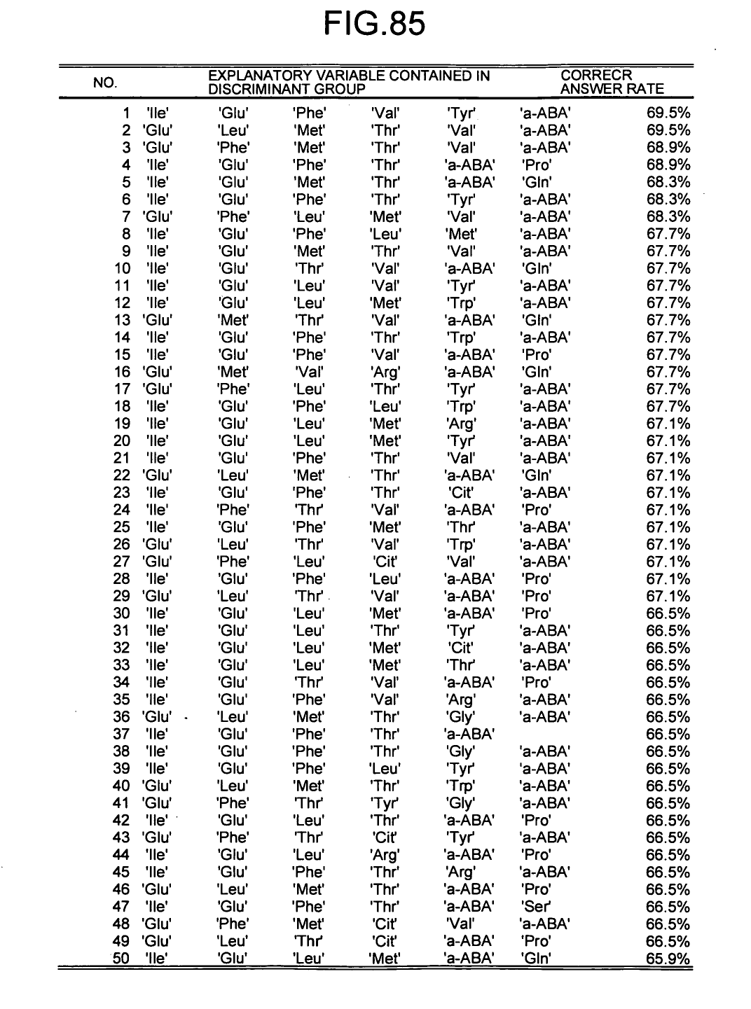

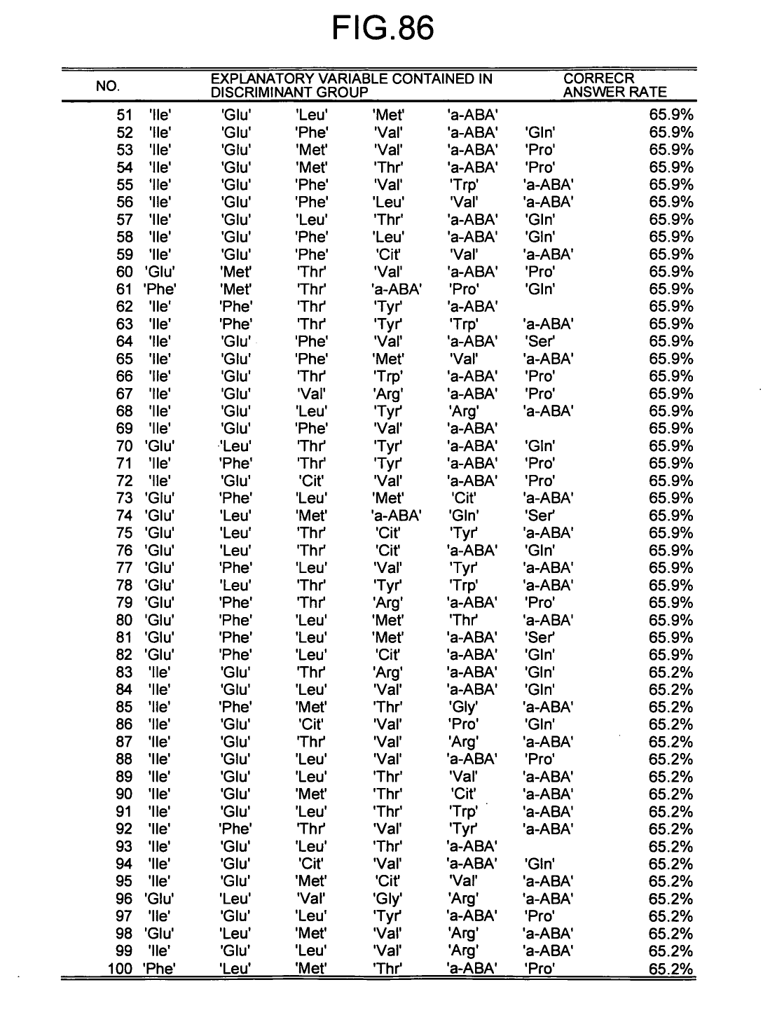

As a result of the evaluation of the diagnosis performance of the colon cancer, the breast cancer, and the cancer-free group based on the index formula group 15 by correct answer rates of discrimination results, high discrimination ability is demonstrated such that the correct answer rates of the cancer-free, the colon cancer, and the breast cancer are 69.6%, 80.0%, and 68.4%, respectively, and the correct answer rate of the total is 70.6% when the prior probability of each is 33.3% (FIG. 84). The value of each coefficient of the discriminant presented in FIG. 83 may be a value obtained by multiplying the same by a real number, and the value of the constant term may be a value obtained by carrying out the addition, subtraction, multiplication or division of an arbitrary real constant to the same. In addition to that, a plurality of discriminant groups having a discrimination performance equivalent to that of the discriminant group presented in FIG. 83 is obtained. The list of the explanatory variables contained in the discriminant groups is presented in FIGS. 85 and 86.

### Example 19

The female data out of the sample data used in Example 14 is used. Indices to maximize performance to discriminate among three groups of the colon cancer group, the breast cancer group, and the cancer-free group are eagerly searched using a method disclosed in International Publication WO 2004/052191 which is an international application filed by the present applicant, and an index formula group 16 composed of the amino acid explanatory variables Thr, Gln, Ala, Cit, ABA, Ile, His, Orn, and Arg is obtained in a plurality of indices having the equivalent performance (FIG. 87).

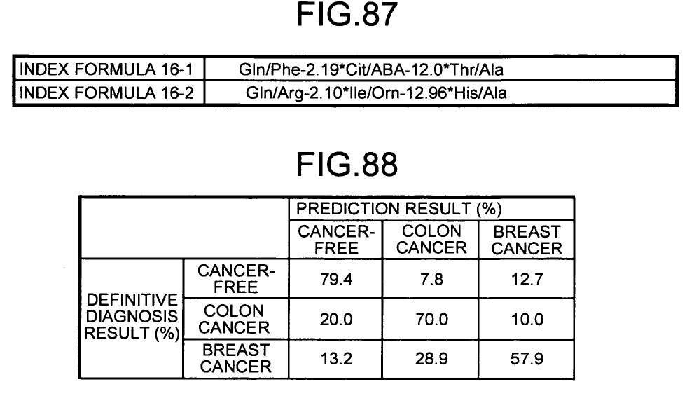

As a result of the evaluation of the diagnosis performance of the colon cancer, the breast cancer, and the cancer-free group based on the index formula group 16 by correct answer rates of discrimination results, high discrimination ability is demonstrated such that the correct answer rates of the cancer-free, the colon cancer, and the breast cancer are 79.4%, 70.0%, and 57.4%, respectively, and the correct answer rate of the total is 73.1% when the prior probability of each is 33.3% (FIG. 88). The value of each coefficient in the discriminant presented in FIG. 87 may be a value obtained by multiplying the same by a real number, and the value of the constant term may be a value obtained by carrying out the addition, subtraction, multiplication or division of an arbitrary real constant to the same.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A method of evaluating cancer type, comprising:
a measuring step of measuring amino acid concentration data on a concentration value of an amino acid in blood collected from a subject to be evaluated; and
a concentration value criterion evaluating step of evaluating a cancer type in the subject based on the concentration value of at least one of Glu, ABA, Val, Met, Pro, Phe, Thr, Ile, Leu, and His contained in the amino acid concentration data of the subject measured at the measuring step.

2. The method of evaluating cancer type according to claim 1, wherein the concentration value criterion evaluating step further includes a concentration value criterion discriminating step of discriminating a cancer in the subject out of at least two of colon cancer, breast cancer, prostatic cancer, thyroid cancer, lung cancer, gastric cancer, and uterine cancer based on the concentration value of at least one of Glu, ABA, Val, Met, Pro, Phe, Thr, Ile, Leu, and His contained in the amino acid concentration data of the subject measured at the measuring step.

3. The method of evaluating cancer type according to claim 2, wherein at the concentration value criterion discriminating step, the cancer in the subject is discriminated out of at least three of colon cancer, breast cancer, prostatic cancer, thyroid cancer, and lung cancer.

4. The method of evaluating cancer type according to claim 1, wherein the concentration value criterion evaluating step further includes:
a discriminant value calculating step of calculating a discriminant value that is a value of a multivariate discriminant with a concentration of the amino acid as an explanatory variable, for each of the multivariate discriminants composing a multivariate discriminant group, based on both (a) the concentration value of at least one of Glu, ABA, Val, Met, Pro, Phe, Thr, Ile, Leu, and His contained in the amino acid concentration data of the subject measured at the measuring step and (b) the multivariate discriminant group composed of one or a plurality of the previously established multivariate discriminants; and
a discriminant value criterion evaluating step of evaluating the cancer type in the subject based on a discriminant value group composed of one or a plurality of the discriminant values calculated at the discriminant value calculating step, and
wherein each of the multivariate discriminants composing the multivariate discriminant group contains at least one of Glu, ABA, Val, Met, Pro, Phe, Thr, Ile, Leu, and His as the explanatory variable.

5. The method of evaluating cancer type according to claim 4, wherein the discriminant value criterion evaluating step further includes a discriminant value criterion discriminating step of discriminating the cancer in the subject out of at least two of colon cancer, breast cancer, prostatic cancer, thyroid cancer, lung cancer, gastric cancer, and uterine cancer based on the discriminant value group.

6. The method of evaluating cancer type according to claim 5, wherein at the discriminant value criterion discriminating step, the cancer in the subject is discriminated out of at least three of colon cancer, breast cancer, prostatic cancer, thyroid cancer, and lung cancer.

7. The method of evaluating cancer type according to claim 6, wherein each of the multivariate discriminants composing the multivariate discriminant group is any one of a fractional expression, a logistic regression equation, a linear discriminant, a multiple regression equation, a discriminant prepared by a support vector machine, a discriminant prepared by a Mahalanobis' generalized distance method, a discriminant prepared by canonical discriminant analysis, and a discriminant prepared by a decision tree.

8. The method of evaluating cancer type according to claim 7, wherein the multivariate discriminant group is any one of following discriminant groups 1 to 16:
discriminant group 1: five linear expressions with age, sex, Thr, Glu, Gln, Pro, Cit, ABA, Val, Met, Ile, Leu, Tyr, Phe, His, Orn, Lys, and Arg as the explanatory variables;
discriminant group 2: four linear expressions with age, Glu, Pro, Cit, ABA, Met, Ile, Leu, Phe, His, Trp, Orn, and Lys as the explanatory variables;
discriminant group 3: four linear expressions with age, Thr, Glu, Gln, Pro, ABA, Val, Met, Ile, Leu, Phe, His, and Arg as the explanatory variables;
discriminant group 4: four linear expressions with age, sex, Thr, Glu, Pro, ABA, Val, Met, Ile, Leu, Phe, and His as the explanatory variables;
discriminant group 5: three linear expressions with age, Asn, Glu, ABA, Val, Phe, His, and Trp as the explanatory variables;
discriminant group 6: three linear expressions with age, Thr, Glu, Pro, Val, Met, Ile, Leu, His, and Arg as the explanatory variables;
discriminant group 7: four linear expressions with age, sex, Thr, Glu, Gln, Pro, Cit, ABA, Val, Met, Ile, Leu, Tyr, Phe, Orn, and Arg as the explanatory variables;
discriminant group 8: three linear expressions with age, Asn, Glu, ABA, Val, Phe, His, and Trp as the explanatory variables;
discriminant group 9: three linear expressions with age, Thr, Glu, Gln, Pro, ABA, Val, Met, Ile, Phe, and Arg as the explanatory variables;
discriminant group 10: three linear expressions with age, sex, Thr, Glu, Pro, ABA, Val, and Met as the explanatory variables;
discriminant group 11: two linear expressions with age, Cit, ABA, Val, and Met as the explanatory variables;
discriminant group 12: two linear expressions with age, Thr, Glu, Pro, Met, and Phe as the explanatory variables;
discriminant group 13: two linear expressions with Thr, Ser, Asn, Glu, Gln, Gly, Ala, Cit, ABA, Val, Met, Ile, Leu, Tyr, Phe, His, Trp, Orn, Lys, and Arg as the explanatory variables;
discriminant group 14: two linear expressions with Glu, Gln, ABA, Val, Ile, Phe, and Arg as the explanatory variables;
discriminant group 15: two linear expressions with Thr, Glu, Gln, ABA, Ile, Leu, and Arg as the explanatory variables; and
discriminant group 16: two fractional expressions with Thr, Gln, Ala, Cit, ABA, Ile, His, Orn, and Arg as the explanatory variables.
